(19) United States
(12) Patent Application Publication
(10) Pub. No.: US 2005/0125054 A1
(43) Pub. Date: Jun. 9, 2005

(54) Devices delivering therapeutic agents and methods regarding the same

(75) Inventors: Vinayak D. Bhat, Santa Clara, CA (US); John Yann, Los Gatos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Avantec Vascular Corporation, Sunnyvale, CA (US)

(21) Appl. No.: 10/993,935

(22) Filed: Nov. 19, 2004

Related U.S. Application Data
(63) Continuation-in-part of application No. 10/607,836, filed on Jun. 27, 2003.
(63) Continuation-in-part of application No. 10/206,807, filed on Jul. 25, 2002.
(63) Continuation-in-part of application No. 10/002,595, filed on Nov. 1, 2001.
(63) Continuation-in-part of application No. 09/783,253, filed on Feb. 13, 2001.
(63) Continuation-in-part of application No. 09/782,927, filed on Feb. 13, 2001, now Pat. No. 6,471,980.
(63) Continuation-in-part of application No. 09/783,254, filed on Feb. 13, 2001.
(63) Continuation-in-part of application No. 09/782,804, filed on Feb. 13, 2001.
Application No. 10/206,807 is a continuation-in-part of application No. 10/017,500, filed on Dec. 14, 2001.
(60) Provisional application No. 60/524,990, filed on Nov. 24, 2003.
(60) Provisional application No. 60/472,536, filed on May 21, 2003.
(60) Provisional application No. 60/454,146, filed on Mar. 11, 2003.
(60) Provisional application No. 60/404,624, filed on Aug. 19, 2002.
(60) Provisional application No. 60/370,703, filed on Apr. 6, 2002.
(60) Provisional application No. 60/355,317, filed on Feb. 7, 2002.
(60) Provisional application No. 60/347,473, filed on Jan. 10, 2002.
(60) Provisional application No. 60/308,381, filed on Jul. 26, 2001.
(60) Provisional application No. 60/258,024, filed on Dec. 22, 2000.

(51) Int. Cl. A61F 2/06

(52) U.S. Cl. 623/1.42

(57) ABSTRACT

Devices and methods for reducing, inhibiting, or treating restenosis and hyperplasia after intravascular intervention are provided. In particular, the present invention provides luminal prostheses which allow for sustained or controlled release of at least one therapeutic capable agent with increased efficacy to selected locations within a patient's vasculature to reduce restenosis. An intraluminal prosthesis may comprise an expandable structure and a source adjacent the expandable structure for releasing the therapeutic capable agent into a body lumen to reduce smooth muscle cell proliferation.

### CROSS-REFERENCES TO RELATED APPLICATIONS

This application claims the benefit of priority from U.S. Provisional Patent Application No. 60/524,990, filed on Nov. 24, 2003. This application is also a continuation-in-part of U.S. patent application Ser. No. 10/607,836, filed on Jun. 27, 2003, which claim benefit of priority from U.S. Provisional Patent Application Nos. 60/472,536, filed on May 21, 2003; 60/454,146, filed on Mar. 11, 2003; 60/404,624, filed on Aug. 19, 2002; and which is a continuation-in-part of U.S. patent application Ser. No. 10/206,807, filed on Jul. 25, 2002, which claims the benefit of priority from U.S. Provisional Patent Application Nos. 60/370,703, filed Apr. 6, 2002, 60/355,317, filed Feb. 7, 2002; and 60/347,473, field Jan. 10, 2002; and which is a continuation-in-part of U.S. patent application Ser. No. 10/002,595, filed on Nov. 1, 2001, which claims the benefit of priority from U.S. Provisional Patent Application No. 60/308,381, filed on Jul. 26, 2001, and which is a continuation-in-part of U.S. patent application Ser. Nos. 09/783,253, 09/782,927 (now U.S. Pat. No. 6,471,980 issued Oct. 29, 2002), Ser. Nos. 09/783,254, 09/782,804 all of which were filed on Feb. 13, 2001 and which claims the benefit of priority from U.S. Provisional Patent Application 60/258,024, filed on Dec. 22, 2000; and which is a continuation-in-part of U.S. patent application Ser. No. 10/017,500, filed on Dec. 14, 2001. Each of the above applications is assigned to the assignee of the present application, the full disclosure of each which is incorporated herein by reference in its entirety. The disclosure of this present application is also related to the disclosures of U.S. patent application Ser. Nos. 10/206,853 and 10/206,803, both filed Jul. 25, 2002, and assigned to the same assignee as that of the present application, the full disclosures of which are incorporated herein by reference in their entirety.

### FIELD OF THE INVENTION

The present invention relates generally to medical devices and methods. More particularly, the present invention relates to luminal prostheses, such as vascular stents and grafts for inhibiting restenosis and hyperplasia.

### BACKGROUND OF THE INVENTION

A number of percutaneous intravascular procedures have been developed for treating stenotic atherosclerotic regions of a patient's vasculature to restore adequate blood flow. The most successful of these treatments is percutaneous transluminal angioplasty (PTA). In PTA, a catheter, having an expandable distal end usually in the form of an inflatable balloon, is positioned in the blood vessel at the stenotic site. The expandable end is expanded to dilate the vessel to restore adequate blood flow beyond the diseased region. Other procedures for opening stenotic regions include directional arthrectomy, rotational arthrectomy, laser angioplasty, stenting, and the like. While these procedures have gained wide acceptance (either alone or in combination, particularly PTA in combination with stenting), they continue to suffer from significant disadvantages. A particularly common disadvantage with PTA and other known procedures for opening stenotic regions is the frequent occurrence of restenosis.

Restenosis refers to the re-narrowing of an artery after an initially successful angioplasty. Restenosis afflicts approximately up to 50% of all angioplasty patients and is the result of injury to the blood vessel wall during the lumen opening angioplasty procedure. In some patients, the injury initiates a repair response that is characterized by smooth muscle cell proliferation referred to as “hyperplasia” in the region traumatized by the angioplasty. This proliferation of smooth muscle cells re-narrows the lumen that was opened by the angioplasty within a few weeks to a few months, thereby necessitating a repeat PTA or other procedure to alleviate the restenosis.

A number of strategies have been proposed to treat hyperplasia and reduce restenosis. Previously proposed strategies include prolonged balloon inflation during angioplasty, treatment of the blood vessel with a heated balloon, treatment of the blood vessel with radiation following angioplasty, stenting of the region, and other procedures. While these proposals have enjoyed varying levels of success, no one of these procedures is proven to be entirely successful in substantially or completely avoiding all occurrences of restenosis and hyperplasia.

As an alternative or adjunctive to the above mentioned therapies, the administration of therapeutic agents following PTA for the inhibition of restenosis has also been proposed. Therapeutic treatments usually entail pushing or releasing a drug through a catheter or from a stent. While holding great promise, the delivery of therapeutic agents for the inhibition of restenosis has not been entirely successful.

Accordingly, it would be a significant advance to provide improved devices and methods for inhibiting restenosis and hyperplasia concurrently with or following angioplasty and/or other interventional treatments. This invention satisfies at least some of these and other needs.

### BRIEF SUMMARY OF THE INVENTION

The present invention provides improved devices and methods for inhibiting stenosis, restenosis, or hyperplasia concurrently with and/or after intravascular intervention. As used herein, the term “inhibiting” means any one of reducing, treating, minimizing, containing, preventing, curbing, eliminating, holding back, or restraining. In particular, the present invention provides luminal prostheses which allow for programmed and sustained or controlled substance delivery with increased efficiency and/or efficacy to selected locations within a patient's vasculature to inhibit restenosis. Moreover, the present invention minimizes drug washout and provides minimal to no hindrance to endothelialization of the vessel wall.

The present invention is directed to improved devices and methods for preparation or treatment of susceptible tissue sites. As used herein, “susceptible tissue site” refers to a tissue site that is injured, or may become injured as a result of an impairment (e.g., disease, medical condition), or may become injured during or following an interventional procedure such as an intravascular intervention. The term “intravascular intervention” includes a variety of corrective procedures that may be performed to at least partially resolve a stenotic, restenotic, or thrombotic condition in a blood vessel, usually an artery, such as a coronary artery. Usually, the corrective procedure will comprise balloon angioplasty. The corrective procedure may also comprise directional atherectomy, rotational atherectomy, laser angioplasty, stenting, or the like, where the lumen of the treated blood vessel is enlarged to at least partially alleviate a stenotic condition which existed prior to the treatment. The susceptible tissue site may include tissues associated with intracorporeal lumens, organs, or localized tumors. In one embodiment, the present devices and methods reduce the formation or progression of restenosis and/or hyperplasia which may follow an intravascular intervention. In particular, the present invention is directed to corporeal, in particular intracorporeal devices and methods using the same.

As used herein, the term “intracorporeal body” refers to body lumens or internal corporeal tissues and organs, within a corporeal body. The “body lumen” may be any blood vessel in the patient's vasculature, including veins, arteries, aorta, and particularly including coronary and peripheral arteries, as well as previously implanted grafts, shunts, fistulas, and the like. It will be appreciated that the present invention may also be applied to other body lumens, such as the biliary duct, which are subject to excessive neoplastic cell growth. Examples of internal corporeal tissue and organ applications include various organs, nerves, glands, ducts, and the like. In one embodiment, the device includes luminal prostheses such as vascular stents or grafts. In another embodiment, the device may include cardiac pacemaker leads or lead tips, cardiac defibrillator leads or lead tips, heart valves, sutures, needles, pacemakers, orthopedic devices, appliances, implants or replacements, or portions of any of the above.

In one embodiment of the present invention, a luminal delivery prosthesis comprises a scaffold which is implantable in a body lumen and means on the scaffold for releasing a substance. The scaffold may be in the form of a stent, which additionally maintains luminal patency, or may be in the form of a graft, which additionally protects or enhances the strength of a luminal wall. The scaffold may be radially expansible and/or self-expanding and is preferably suitable for luminal placement in a body lumen. An exemplary stent for use in the present invention is described in co-pending U.S. patent application Ser. No. 09/565,560, assigned to the assignee of the present application, the full disclosure of which is incorporated herein by reference.

In one embodiment, the devices and methods of the present invention inhibit the occurrence of restenosis while allowing for the generation of small amount of cellularization, endothelialization, or neointima, preferably, in a controlled manner. “Restenosis” in this instance is defined as when the artery narrows greater than about 40% to about 80% of the acute vessel diameter achieved by the vascular intervention, such as stenting, usually from about 50% to about 70%.

In an embodiment, the device includes a structure and at least one source of at least one therapeutic capable agent associated with the structure. As used herein the term “associated with” refers to any form of association such as directly or indirectly being coupled to, connected to, disposed on, disposed within, attached to, adhered to, bonded to, adjacent to, entrapped in, absorbed in, absorbed on, and like configurations. The therapeutic capable agent source is associated at least in part with the structure in a manner as to become available, immediately or after a delay period, to the susceptible tissue site upon introduction of the device within or on the corporeal body. In an embodiment, the source may be disposed or formed adjacent at least a portion of the structure. In one embodiment, the source may be disposed or formed adjacent at least a portion of either or both surfaces of the expandable structure, within the interior of the structure disposed between the two surfaces, or any combination thereof. In one embodiment, the source may be disposed only on one of the longitudinal surfaces, namely, the tissue facing surface. The association of the therapeutic capable agent with the structure may be continuous or in discrete segments. In an embodiment, the structure may be an expandable structure. In another embodiment, the structure may have a substantially constant size or diameter, or alternatively depending on the application and use, may be a contractable structure. In an embodiment, the structure includes at least one surface, usually, a tissue facing surface (i.e., abluminal surface). In another embodiment, the structure includes an abluminal surface and another surface, usually a lumen facing surface. In an embodiment, the structure may have an interior disposed between two luminal and abluminal surfaces.

The device may be implantable within a corporeal body which includes the susceptible tissue site or may be configured for implanting, with or without expansion, at a targeted corporeal site. The targeted corporeal site may include the susceptible tissue site or may be another corporeal site (e.g., other body organs or lumens). For example, the corporeal site may comprise the targeted intracorporeal site, such as an artery, which supplies blood to the susceptible tissue site. In an embodiment, the expandable structure may be in the form of a stent, which additionally maintains luminal patency, or in the form of a graft, which additionally protects or enhances the strength of a luminal wall. The device, may comprise at least in part, a scaffold formed from an open lattice or an at least substantially closed surface. In an embodiment, the stent comprises a scaffold formed at least in part from an open lattice. The expandable structure may be radially expandable and/or self-expanding and is preferably suitable for luminal placement in a body lumen.

The expandable structure may be formed of any suitable material such as metals, polymers, or a combination thereof. In one embodiment, the expandable structure may be formed of an at least partially biodegradable material selected from the group consisting of polymeric material, metallic materials, or combinations thereof. The at least partially biodegradable material preferably degrades over time. Examples of polymeric material include poly-L-lactic acid, having a delayed degradation to allow for the recovery of the vessel before the structure is degraded. Examples of metallic material include metals or alloys degradable in the corporeal body, such as stainless steel.

In one embodiment, the luminal prosthesis makes available one or more therapeutic capable agents to one or more selected locations within a patient's vasculature, including the susceptible tissue site, to reduce the formation or progression of restenosis and/or hyperplasia. As used herein, the term “made available” means to have provided the substance (e.g., therapeutic capable agent) at the time of release or administration, including having made the substance available at a corporeal location such as an intracorporeal location or target site, regardless of whether the substance is in fact delivered, used by, or incorporated into the intended site, such as the susceptible tissue site.

The delivery of the therapeutic capable agent to the susceptible tissue site, or making the therapeutic capable agent available to the susceptible tissue site, may be direct or indirect through another corporeal site. In the latter embodiment, the another corporeal site is a targeted intracorporeal site, for example an intracorporeal lumen, such as an artery, supplying blood to the susceptible tissue site.

As used herein, “therapeutic capable agent” includes at least one compound, molecular species, and/or biologic agent that is either therapeutic as it is introduced to the subject under treatment, becomes therapeutic after being introduced to the subject under treatment as for example by way of reaction with a native or non-native substance or condition, or another introduced substance or condition. Examples of native conditions include pH (e.g., acidity), chemicals, temperature, salinity, osmolality, and conductivity; with non-native conditions including those such as magnetic fields, electromagnetic fields (such as radiofrequency and microwave), and ultrasound. In the present application, the “chemical name” of any of the therapeutic capable agents or other compounds is used to refer to the compound itself and to pro-drugs (precursor substances that are converted into an active form of the compound in the body), and/or pharmaceutical derivatives, analogues, or metabolites thereof (bio-active compound to which the compound converts within the body directly or upon introduction of other agents or conditions (e.g., enzymatic, chemical, energy), or environment (e.g., pH)).

The therapeutic capable agent may be selected from a group consisting of immunosuppressants, anti-inflammatories, anti-proliferatives, anti-migratory agents, anti-fibrotic agents, proapoptotics, vasodilators, calcium channel blockers, anti-neoplastics, anti-cancer agents, antibodies, anti-thrombotic agents, anti-platelet agents, Ib/IIIa agents, antiviral agents, MTOR (mammalian target of rapamycin) inhibitors, non-immunosuppressant agents, tyrosine kinase inhibitors, CDK inhibitors, bisphosphonates, NF-κB Decoy Oligo, proteins, oligomers, amino acids, peptides, genes, growth factors, anti-sense and a combination thereof. Specific examples of therapeutic capable agent include: mycophenolic acid, mycophenolic acid derivatives (e.g., 2-methoxymethyl derivative and 2-methyl derivative), VX-148, VX-944, mycophenolate mofetil, mizoribine, methylprednisolone, dexamethasone, CERTICAN™ (e.g., everolimus, RAD), rapamycin, ABT-578, ABT-773 (Abbot Labs), ABT-797 (Abbot Labs), TRIPTOLIDE™, METHOTREXATE™, phenylalkylamines (e.g., verapamil), benzothiazepines (e.g., diltiazem), 1,4-dihydropyridines (e.g., benidipine, nifedipine, nicarrdipine, isradipine, felodipine, amlodipine, nilvadipine, nisoldipine, manidipine, nitrendipine, bamidipine (HYPOCA™)), ASCOMYCIN™, PIMECROLIMUS™, WORTMANNIN™, LY294002, CAMPTOTHECIN™, silibinin, sylymarin, baicalein, histone deacetylase such as trichostatin A, PD-0183812, butyrolactone I, substituted purines (e.g., olomoucine, CGP74514, and its derivatives), polyhydroxylated flavones (e.g., flavopyridol), oxindole inhibitors (e.g., GW-8510, GW-2059, GW-5181), and indolinone derivatives (e.g., SU-5416),), Zoledronic acid (i.e., ZOMETA™, Zoledronic acid, and (1-Hydroxy-2-imidazol-1-yl-phosphonoethyl) phosphonic acid monohydrate), isoquinoline, HA-1077 (1-(5-isoquinolinesulfonyl)-homopiperazine hydrochloride), TAS-301 (3-bis(4-methoxyphenyl)methylene-2-indolinone), TOPOTECAN™, hydroxyurea, TACROLIMUS™ (FK 506), cyclophosphamide, cyclosporine, daclizumab, azathioprine, prednisone, diferuloymethane, diferuloylmethane, diferulylmethane, GEMCITABINE™, cilostazol (PLETAL™), TRANILAST™, enalapril, quercetin, suramin, estradiol, cycloheximide, tiazofurin, zafurin, AP23573, rapamycin derivatives, non-immunosuppressive analogues of rapamycin (e.g., rapalog, SAR943 (32-deoxorapamycin), AP21967, derivatives of rapalog, SAR943 (32-deoxorapamycin)), CCI-779 (an analogue of rapamcin available from Wyeth), sodium mycophenolic acid, benidipine hydrochloride, sirolimus, rapamune, phenylaminopyrimidine (or phenylpyrimidine-amine) derivatives (e.g., Imatinib (GLIVEC™)), other tyrosine inhibitors such as 4-[6-methoxy-7-(3-piperidine-1-yl-propoxy)-quinazolin-4-yl]-piperazine-1-carboxylicacid(4-isopropoxyphenyl) amide (CT53518 or MLN518 from Millennium Pharmaceutical), 5-Chloro-3-[(3,5-dimethylpyrrol-2-yl)methylene]-2-indolinone (SU6656), 5-Chloro-3-[(3,5-dimethylpyrrol-2-yl)methylene]-2-indolinone (SU5614 from Sugen), a water-soluble N,N-dimethylgly-cine ester prodrug CEP7055 that converts to CEP5214 in vivo from Cephalon, West Chester Pa., 4-Amino-5-(4-chlorophenyl)-7-(t-butyl)pyrazolo[3,4-d]pyrimidine (PP2 or AG1879), 6,7-Dimethyl-2-phenylquinoxaline (AG1295), TAUTOMYCIN™, Radicicol, Damnacanthal, Herbimycin A, 6-(2,6-dichloro-phenyl)-8-methyl-2-(3-methylsulfanyl-phenylamino)-8h-pyrido(2,3-d)pyrimidin-7-one (PD173955 from Parke-Davis), PD166326, PD183805, 4-[(3-Bromophenyl)amino]-6-propionylamidoquinazoline (PD174265), 5-Chloro-3-[(3,5-dimethylpyrrol-2-yl)methylene]-2-indolinone (PD153035), 4-[(3-Bromophenyl)amino]-6-acrylamidoquinazoline (PD168393), TARCEVA™ (erlotinib HCl), CI-1033, AEE788, CP-724,714 (from OSI Pharmaceutical), Geldanamycin, 17-(allylamino)-17-demethoxygeldanamycin (17-AG or 12-AAG), TARCEVA™, IRESSA™, and ZD4910, EGFR/ErbB2 inhibitor (CI1033; EKB569; GW2016; PKI166), VEGF receptor inhibitors (ZK222584;ZD6474), VEGFR/FGFR/PDGFR inhibitors (SU6668; SU11248; PTK787), NGF receptor inhibitors (CEP2583), anti-EGF receptor MAbs (MAb225/ERBITUX™), anti-ErbB2 MAbs (MAb4D5/HERCEPTIN™), AVASTIN™, an anti-VEGF MAb, NF-κB Decoy Oligo, albumin, TSC1, TSC2, hamartin KIAA0243, VEGF, EGF, PDGF, FGF, Antisense phosphorothioate oligodeoxynucleotide (ODN), Anti-MTOR, Anti-p27 Anti-p53, Anti-Cdk, metabolites, derivatives, agent incorporated in a vector such as a HVJ Envelop vector, IGF, IGF-1, IGFBP, IGFBP-3, rhlGFBP-3, and/or combinations thereof.

In an embodiment, the source of the therapeutic capable agent is a polymeric material including therapeutic capable agent moieties as a structural subunit of the polymer. The therapeutic capable agent moieties are polymerized and associated to one another through suitable linkages (e.g., ethylenic) forming polymeric therapeutic capable agent. Once the polymeric therapeutic capable agent is brought into contact with tissue or fluid such as blood, the polymeric therapeutic capable agent subunits disassociate. Alternatively, the therapeutic capable agent may be released as the polymeric therapeutic capable agent degrades or hydrolyzes, preferably, through surface degradation or hydrolysis, making the therapeutic capable agent available to the susceptible tissue site, preferably over a period of time. Examples of methods and compounds for polymerizing therapeutic capable agents are described in WO 99/12990 Patent Application by Kathryn Uhrich, entitled “Polyanhydrides With Therapeutically Useful Degradation Products,” and assigned to Rutgers University, the full disclosure of which is incorporated herein by reference. Examples of a therapeutic capable agent and a suitable reaction ingredient unit include mycophenolic acid with adipic acid and/or salicylic acid in acid catalyzed esterification reaction, mycophenolic acid with aspirin and/or adipic acid in acid catalyzed esterification reaction, mycophenolic acid with other NSAIDS, and/or adipic acid in acid catalyzed esterification reaction. In an embodiment, the polymeric therapeutic capable agent may be associated with a polymeric and/or metallic backbone, wherein the therapeutic capable agent units are disassociated over time in the corporeal body or vascular environment.

The devices of the present invention may be configured to release or make available the therapeutic capable agent at one or more phases, the one or more phases having similar or different performance (e.g., release) profiles. The therapeutic capable agent may be made available to the tissue at amounts which may be sustainable, intermittent, or continuous; in one or more phases; and/or rates of delivery; effective to reduce any one or more of smooth muscle cell proliferation, inflammation, immune response, hypertension, or those complementing the activation of the same.

In one embodiment, the substance is released over a predetermined time pattern comprising an initial phase wherein the substance delivery rate is below a threshold level and a subsequent phase wherein the substance delivery rate is above a threshold level. The predetermined time pattern of the present invention improves the efficiency of drug delivery by releasing a lower or minimal amount of the substance until a subsequent phase is reached, at which point the release of the substance may be substantially higher. Thus, time delayed substance release can be programmed to impact restenosis substantially at the onset of events leading to smooth muscle cell proliferation (hyperplasia). The present invention can further minimize substance washout by timing substance release to occur after at least initial cellularization and/or endothelialization which creates a barrier over the stent to reduce loss of the substance directly into the bloodstream. Moreover, the predetermined time pattern may reduce substance loading and/or substance concentration as well as potentially providing minimal to no hindrance to endothelialization of the vessel wall due to the minimization of drug washout and the increased efficiency of substance release. Any one of the at least one therapeutic capable agents may perform one or more functions, including preventing or reducing proliferative/restenotic activity, reducing or inhibiting thrombus formation, reducing or inhibiting platelet activation, reducing or preventing vasospasm, or the like. The devices may be configured to make available to the tissue the most suitable therapeutic amount of the therapeutic capable agent while minimizing the presence of unwanted metabolites and by-products of the therapeutic capable agent at the tissue site.

The total amount of therapeutic capable agent made available to the tissue depends in part on the level and amount of desired therapeutic result. The therapeutic capable agent may be made available at one or more phases, each phase having similar or different release rate and duration as the other phases. The release rate may be pre-defined. In an embodiment, the rate of release may provide a sustainable level of therapeutic capable agent to the susceptible tissue site. In another embodiment, the rate of release is substantially constant. The rate may decrease and/or increase over time, and it may optionally include a substantially non-release period. The release rate may comprise a plurality of rates. In an embodiment the plurality of release rates include at least two rates selected from the group consisting of substantially constant, decreasing, increasing, substantially non-releasing.

The total amount of therapeutic capable agent made available or released may be in an amount ranging from about 0.1 μg (micrograms) to about 10 g (grams), generally from about 0.1 μg to about 10 mg (milligrams), usually from about 1 μg to about 10 mg, from about 1 μg to about 5 mg, from about 1 μg to about 2 mg, from about 10 μg to about 2 mg, from about 10 μg to about 1 mg, from about 50 μg to about 1 mg, or from about 50 μg to about 500 μg. In an embodiment, the therapeutic capable agent may be released in a time period, as measured from the time of implanting of the device, ranging from about 1 day to about 200 days; from about 1 day to about 45 days; or from about 7 days to about 21 days. In an embodiment the release rate of the therapeutic capable agent per day may range from about 0.001 μg to about 500 μg, from about 0.001 μg to about 200 μg, from about 0.5 μg to about 200 μg, usually, from about 1.0 μg to about 100 μg, from about 1 μg to about 60 μg, and typically, from about 5 μg to about 50 μg.

The therapeutic capable agent may be made available at an initial phase and one or more subsequent phases. When the therapeutic capable agent is delivered at different phases, the initial delivery rate will typically be from about 0 to about 99% of the subsequent release rates, usually from about 0% to about 90%, preferably from about 0% to 75%, more preferably from about 0% to 50%. The rate of delivery during the initial phase will typically range from about 0.001 ng (nanograms) per day to about 500 μg per day, from about 0 to about 50 μg per day, usually from about 0.001 ng per day to about 50 μg per day, more usually from about 0.1 μg per day to about 30 μg per day, more preferably, from about 1 μg per day to about 20 μg per day. The rate of delivery at the subsequent phase may range from about 0.01 ng per day to about 500 μg per day, from about 0.01 μg per day to about 200 μg per day, usually from about 1 μg per day to about 100 μg per day. In one embodiment, the therapeutic capable agent is made available to the susceptible tissue site in a programmed, sustained, and/or controlled manner with increased efficiency and/or efficacy. Moreover, the present invention provides limited or reduced hindrance to endothelialization of the vessel wall. Further, the release rates may vary during either or both of the initial and subsequent release phases. There may also be additional phase(s) for release of the same substance(s) and/or different substance(s).

The duration of the initial, subsequent, and any other additional phases may vary. For example, the release of the therapeutic capable agent may be delayed from the initial implantation of the device. Typically, the delay is sufficiently long to allow the generation of sufficient cellularization or endothelialization at the treated site to inhibit loss of the therapeutic capable agent into the vascular lumen. Typically, the duration of the initial phase will be sufficiently long to allow initial cellularization or endothelialization of at least part of the device. Typically, the duration of the initial phase, whether being a delayed phase or a release phase, is less than about 24 weeks, from about 1 hour to about 24 weeks, usually less than about 12 weeks, more usually from about 1 hour to about 8 weeks, from about 1 day to about 30 days, from about 12 hours to about 4 weeks, from about 12 hours to about 2 weeks, from about 1 day to about 2 weeks, or from about 1 day to about 1 week.

The durations of the one or more subsequent phases may also vary, typically being from about 4 hours to about 24 weeks, from about 1 hour to about 12 weeks, from about 1 day to about 12 weeks, from about 1 hour to about 8 weeks, from about 4 hours to about 8 weeks, from about 2 days to about 8 weeks, from about 2 days to about 45 days, from about of 3 days to about 50 days, from about 3 days to about 30 days, from about 1 hour to about 1 day. In an embodiment, the duration specified relates to a vascular environment. The more than one phase may include similar or different durations, amounts, and/or rates of release. For example, in one scenario, there may be an initial phase of delay, followed by a subsequent phase of release at a first subsequent rate, and a second subsequent phase of release at a second subsequent rate, and the like.

In an embodiment a mammalian tissue concentration of the substance at an initial phase will typically be within a range from about 0.001 ng/mg of tissue to about 100 μg/mg of tissue; from about 1 ng/mg of tissue to about 100 μg/mg of tissue; from about 10 ng/mg of tissue to about 100 μg/mg of tissue; from about 0.1 ng/mg of tissue to about 50 μg/mg of tissue; from about 1 ng/mg of tissue to about 10 μg/mg of tissue; from about 1 ng/mg of tissue to about 1 μg/mg of tissue. A mammalian tissue concentration of the substance at a subsequent phase will typically be within a range from about 0.001 ng/mg of tissue to about 600 μg/mg of tissue, preferably from about 0.001 ng/mg of tissue to about 100 μg/mg of tissue, from about 0.1 ng/mg of tissue to about 10 μg/mg of tissue, from about 1 ng/mg of tissue to about 10 μg/mg of tissue.

Alternatively, the device of the present invention may be configured to deliver the therapeutic capable agent at a phase to a susceptible tissue site of a mammalian intracorporeal body to effectuate a mammalian tissue concentration ranging from about 0.001 ng of therapeutic capable agent/mg of tissue to about 100 μg of therapeutic capable agent/mg of tissue, usually from about 1 ng of therapeutic capable agent/mg of tissue to about 100 μg of therapeutic capable agent/mg of tissue, preferably from about 1 ng of therapeutic capable agent/mg of tissue to about 10 μg of therapeutic capable agent/mg of tissue, more preferably from about 0.15 ng of therapeutic capable agent/mg of tissue to about 3 ng of therapeutic capable agent/mg of tissue. The therapeutic capable agent as administered, may be converted to metabolites which may or may not be desirable. In an embodiment, the mammalian tissue concentration of the undesirable metabolite of the therapeutic capable agent, such as metabolite of mycophenolic acid (phenolic glucuronide of MYCOPHENOLIC ACID, MPAG), is less than about 250 ng/100 mg of tissue, normally, less than about 110 ng/100 mg of tissue, usually less than about 50 ng/100 mg of tissue, desirably less than about 25 ng/100 mg of tissue, more preferably, less than about 10 ng/100 mg of tissue, and most desirably substantially zero.

In an embodiment, the device further includes an optional another compound, such as another therapeutic capable agent, or another compound enabling and/or enhancing either or both the release and efficacy of the therapeutic capable agent. The another therapeutic capable agent may be associated with expandable structure in the same or different manner as the first therapeutic capable agent. The another therapeutic capable agent may act in synergy with the therapeutic capable agent, in ways such as compensating for the possible reactions and by-products that can be generated by the therapeutic capable agent. By way of example, the therapeutic capable agent may reduce generation of desired endothelial cells while a suitable another therapeutic capable agent may allow for more endothelialization to be achieved. The another therapeutic agent may be released prior to, concurrent with, or subsequent to, the therapeutic capable agent, at similar or different rates and phases.

The another therapeutic capable agent may comprise at least one compound selected from the group consisting of anti-cancer agents; chemotherapeutic agents; thrombolytics; vasodilators; antimicrobials or antibiotics antimitotics; growth factor antagonists; free radical scavengers; biologic agents; radiotherapeutic agents; radiopaque agents; radiolabelled agents; anti-coagulants such as heparin and its derivatives; anti-angiogenesis drugs such as THALIDOMIDE™; angiogenesis drugs; PDGF-B and/or EGF inhibitors; anti-inflamatories including psoriasis drugs; riboflavin; tiazofurin; zafurin; anti-platelet agents including cyclooxygenase inhibitors such as acetylsalicylic acid; ADP inhibitors such as clopidogrel (e.g., PLAVIX™) and ticlopdipine (e.g., TICLID™); phosphodiesterase III inhibitors such as cilostazol (e.g., PLETAL™); glycoprotein IIb/IIIa agents such as abciximab (e.g., RHEOPRO™); eptifibatide (e.g., INTEGRILIN™); and adenosine reuptake inhibitors such as dipyridmoles; healing and/or promoting agents including anti-oxidants; nitrogen oxide donors; antiemetics; antiauseants; CDK inhibitors, bisphosphonates, NF-κB Decoy Oligo; proteins such as albumin; genes such as TSC1, TSC2, hamartin, or KIAA0243; growth factors such as VEGF, EGF, PDGF, or FGF; anti-sense such as Antisense phosphorothioate oligodeoxynucleotide (ODN); anti-bodies such as Anti-MTOR, Anti-p27 Anti-p53, or Anti-Cdk; derivatives, agent incorporated in a vector such as a HVJ Envelop vector; derivatives and combinations thereof.

In an embodiment, the another compound comprises, an enabling compound responsive to an external form of energy, or native condition, to effect or modify the release of the therapeutic capable agent. The responsive compound may be associated with the therapeutic capable agent, a rate-sustaining or rate-controlling element, the expandable structure, or a combination thereof. The second enabling compound may be formed from magnetic particles coupled to the therapeutic capable agent. The energy source may be a magnetic source for directing a magnetic field at the prosthesis after implantation to effect release of the therapeutic capable agent.

In an embodiment, the device further includes a rate-sustaining or rate-controlling element for affecting the rate of release of the therapeutic capable agent and/or the another compound. In an embodiment, the rate-sustaining or rate-controlling element may be disposed or formed adjacent the structure. In one embodiment, the rate-sustaining or rate-controlling element may be disposed or formed adjacent at least a portion of the optional one or more surfaces of the structure (e.g., luminal or abluminal surfaces), or within the optional interior of the structure, or any combination thereof. The therapeutic capable agent or the optional another compound may be disposed adjacent the rate-sustaining or rate-controlling element. Additionally and/or alternatively, in one embodiment, the therapeutic capable agent or the optional another compound may be mixed with the rate-sustaining or rate-controlling element forming a matrix therewith. In an embodiment, the therapeutic capable agent or the optional another compound itself is a rate-sustaining or rate-controlling element, as for example, when the therapeutic capable agent or the optional another compound is a polymeric material.

The term “matrix” as used herein refers to an association between the rate-sustaining or rate-controlling element and the therapeutic capable agent (or the optional another compound) and/or any other compounds or structures affecting the release of the therapeutic capable agent and the therapeutic capable agent (or the optional another compound). In an embodiment, the matrix is formed as a matrix interface between the rate-sustaining or rate-controlling element and the therapeutic capable agent and/or the optional another compound. In an embodiment, the rate-sustaining or rate-controlling element may comprise multiple adjacent layers formed from the same or different material. The therapeutic capable agent or the optional another compound may be present adjacent one or more of the rate-sustaining or rate-controlling element layers. Additionally and/or alternatively, the therapeutic capable agent or the optional another compound may form a matrix and/or matrix interface with one or more of the rate-sustaining or rate-controlling element layers.

In another embodiment, when the rate-sustaining or rate-controlling element is present as multiple layers, any one of the more than one layers may include independently none, one, or more of the plurality of compounds (e.g., the at least one therapeutic capable agent, another compound). Each of the plurality of compounds such as the another compound and/or more than one therapeutic capable agent, may form a different matrix with the rate-sustaining or rate-controlling element. In an embodiment, as further described below, the first therapeutic capable agent may form the matrix, as when the therapeutic capable agent is a polymeric therapeutic capable agent, thus sustaining or controlling the release of an active component to the susceptible tissue site. Alternatively, or additionally, the rate-sustaining or rate-controlling element may be another compound, such as another therapeutic capable agent which can have an impact on the release rate of the first therapeutic capable agent.

The rate-sustaining or rate-controlling element may be formed of a non-degradable, partially degradable, substantially degradable material, or a combination thereof. The material may be synthetic or natural; non-polymeric, polymeric or metallic; bio-active or non bio-active compounds; or a combination thereof. By way of examples, a metallic material that at least partially degrades with time may be used as the rate-sustaining or rate-controlling element; as well as non-polymers having large molecular weight, polar or non-polar functional groups, electrical charge, steric hindrance groups, hydrophobic, hydrophilic, or amphiphilic moieties.

Suitable biodegradable rate-sustaining or rate-controlling element materials include, but are not limited to, poly(lactic acid), poly(glycolic acid) and copolymers, poly dioxanone, poly(ethyl glutamate), poly(hydroxybutyrate), polyhydroxyvalerate and copolymers, polycaprolactone, polyanhydride, poly(ortho esters), poly(iminocarbonates), polycyanoacrylates, polyphosphazenes, polyester-amids, copolymers and other aliphatic polyesters, or suitable copolymers thereof including copolymers of poly-L-lactic acid and poly-e-caprolactone, and mixtures, copolymers, and combinations thereof. Other suitable examples of biodegradable rate-sustaining or rate-controlling element include polyamide esters made from amino acids (such as L-lysine and l-leucine) along with other building blocks such as diols (hexanediol) and diacids (such as sebacic acid, as described in another embodiment). The therapeutic capable agent may be released either from a reservoir or a matrix comprising the above polymer. The therapeutic capable agent may be also covalently attached to the amino acids and released as the polymer biodegrades. Other biodegradable poly ester urethanes made from copolymers of poly lactide, poly caprolactone, poly ethylene glycol, polyester-amids, and poly acrylic acid can also be used to release the therapeutic capable agent as described above.

Suitable nondegradable or slow degrading rate-sustaining or rate-controlling element materials include, but are not limited to, polyurethane, polyethylene, polyethylenes imine, cellulose acetate butyrate, ethylene vinyl alcohol copolymer, silicone, polytetrafluorethylene (PTFE), parylene, parylene C, N, D, or F, non-porous parylene C, PARYLAST™, PARYLAST™ C, poly(methyl methacrylate butyrate), poly-N-butyl methacrylate, poly(methyl methacrylate), poly 2-hydroxy ethyl methacrylate, poly ethylene glycol methacrylates, poly vinyl chloride, poly(dimethyl siloxane), poly(tetrafluoroethylene), poly(ethylene oxide), poly ethylene vinyl acetate, poly carbonate, poly acrylamide gels, N-vinyl-2-pyrrolidone, maleic anhydride, Nylon, cellulose acetate butyrate (CAB) and the like, including other synthetic or natural polymeric substances, and mixtures, copolymers, and combinations thereof. These polymers can have a foam structure, porous structure, nano-porous structure, non-porous structure, structure with cracks, openings, fissures, perforations or combinations thereof. In an embodiment the rate-sustaining or rate-controlling element is formed from a material selected from the group consisting of silicone, polytetrafluoroethylene, parylene, parylene C, non-porous parylene C, PARYLAST™, PARYLAST™ C, polyurethane, cellulose acetate butyrate, and mixtures, copolymers and combinations thereof.

Suitable natural materials include, but are not limited to, fibrin, albumin, collagen, gelatin, glycosoaminoglycans, oligosaccharides & poly saccharides, chondroitin, phosholipids, phosphorylcholine, glycolipids, proteins, oligomers, amino acids, peptides, cellulose, and mixtures, copolymers, or combinations thereof. Other suitable materials include, titanium, chromium, Nitinol, gold, stainless steel, metal alloys, or a combination thereof as well as other compounds that may release the therapeutic capable agent as a result of interaction (e.g., chemical reaction, high molecular weight, steric hindrance, hyrophobicity, hydrophilicity, amphilicity, heat) of the therapeutic capable agent with the rate-sustaining or rate-controlling element material (e.g, a non-polymer compound). By way of example, a combination of two or more metals or metal alloys with different galvanic potentials to accelerate corrosion by galvanic corrosion pathways may also be used.

The degradable material may degrade by bulk degradation or hydrolysis. In an embodiment, the rate-sustaining or rate-controlling element degrades or hydrolyzes throughout, or preferably, by surface degradation or hydrolysis, in which a surface of the rate-sustaining or rate-controlling element degrades or hydrolyzes over time while maintaining bulk integrity. In another embodiment, hydrophobic rate-sustaining or rate-controlling elements are preferred as they tend to release therapeutic capable agent at desired release rate. A non-degradable rate-sustaining or rate-controlling element may release therapeutic capable agent by diffusion. By way of example, if the rate-sustaining or rate-controlling element is formed of non-polymeric material, the therapeutic capable agent may be released as a result of the interaction (e.g., chemical reaction, high molecular weight, steric hindrance, hyrophobicity, hydrophilicity, amphilicity, heat) of the therapeutic capable agent with the rate-sustaining or rate-controlling element material (e.g, a non-polymer compound). In an embodiment, when the rate-sustaining or rate-controlling element does not form, at least a sufficient matrix with the therapeutic capable agent, the therapeutic capable agent may be released by diffusion through the rate-sustaining or rate-controlling element.

The rate-sustaining or rate-controlling element may have a sufficient thickness so as to provide the desired release rate of the therapeutic capable agent. The rate-sustaining or rate-controlling element will typically have a total thickness in a range from about 10 nm to about 100 μm. The thickness may also range from about 50 nm to about 100 μm, from about 100 nm to about 50 μm, or from about 100 nm to 10 μm.

Vapor and plasma deposited coating are well suited for agents such as NF-κB Decoy Oligo, proteins, oligomers, amino acids, peptides, genes, anti-sense, growth factors, anti-bodies, or combination thereof because these coatings can be applied at room temperature and without the use of a solvent. The use of solvent or higher temperature for coating application affects these agents by causing denaturing, degradation, or the like. As a result, the drug loses some or all of its potency and functionality.

The therapeutic capable agent may be associated with either or both the structure (e.g., expandable structure) and the rate-sustaining or rate-controlling element in any one or more ways as described above. The therapeutic capable agent may be disposed adjacent (e.g., on or within) the expandable structure. Alternatively or additionally, the therapeutic capable agent may be disposed adjacent (e.g., on or within) the rate-sustaining or rate-controlling element, or in an interface between the structure and the rate-sustaining or rate-controlling element, in a pattern that provides the desired performance (e.g., release rate). In an embodiment, the device includes an outer layer including the therapeutic capable agent. In an embodiment, the therapeutic capable agent outer layer provides for a bullous release (e.g., an initial release) of the therapeutic capable agent upon introduction of the device to the corporeal body.

In yet another embodiment the therapeutic capable agent is made available to the susceptible tissue site as a native environment of the area where the device is implanted changes. For example, a change in a pH of the area where the device is implanted may change over time so as to bring about the release of the therapeutic capable agent directly (i.e. when a polymeric drug acts as the matrix including both the therapeutic capable agent and the rate-sustaining or rate-controlling element), or indirectly by affecting the erosion or diffusion characteristic of the rate-sustaining or rate-controlling element as either or both the matrix or non-matrix. For example, as the pH increases or decreases, the erosion of the rate-sustaining or rate-controlling element changes allowing for initial and subsequent phase releases.

The source may be associated with at least a portion of the structure (e.g., prosthesis) using coating methods such as spraying, dipping, deposition (vapor or plasma), painting, and chemical bonding. Such coatings may be uniformly or intermittently applied to the structure or may be applied in a random or pre-determined pattern. In an embodiment, when the structure includes one or more surfaces and optional interior between the surfaces, the coating may be applied to only one of the surfaces of the prosthesis or the coating may be thicker on one side. Furthermore, a biocompatible (e.g., blood compatible) layer may be formed over the source and/or the most outer layer of the device, to make or enhance the biocompatibility of the device. Suitable biocompatible materials for use as the biocompatible layer include, but are not limited to, polyethylene glycol (PEG), polyethylene oxide (PEO), hydrogels, silicone, polyurethanes, and heparin coatings.

In another embodiment, the surface of the structure may be pre-processed using any of a variety of procedures, including, cleaning; physical modifications such as etching or abrasion; and chemical modifications such as solvent treatment, the application of primer coatings, the application of surfactants, plasma treatment, ion bombardment, and covalent bonding. In an embodiment, a metal film or alloy with a small pit(s) or pin hole(s) to accelerate corrosion by pitting corrosion, allows the pin hole formed by the corrosion to act as an orifice for drug release. In an embodiment, the therapeutic capable agent may be attached to the metal or metal alloy.

When the device includes the source including a plurality of compounds (e.g., first therapeutic capable agent and an another compound such as another or second therapeutic capable agent or enabling compound), the plurality of compounds may be released at different times and/or rates, from the same or different layers. Each of the plurality of compounds may be made available independently of one another (e.g., sequential), simultaneous with one another, or concurrently with and/or subsequent to the interventional procedure. For example, a first therapeutic capable agent (e.g., TRIPTOLIDE™) may be released within a time period of 1 day to 45 days with the second therapeutic capable agent (e.g, mycophenolic acid) released within a time period of 2 days to 3 months, from the time of interventional procedure.

The devices of the present invention may be provided together with instructions for use (IFU), separately or as part of a kit. The kit may include a pouch or any other suitable package, such as a tray, box, tube, or the like, to contain the device and the IFU, where the IFU may be printed on a separate sheet or other media of communication and/or on the packaging itself. In an embodiment, the kit may also include a mounting hook, such as a crimping device and/or an expansible inflation member, which may be permanently or releaseably coupled to the device of the present invention. In an embodiment, the kit may comprise the device and an IFU regarding use of a second compound prior to, concurrent with, or subsequent to, the interventional procedure or first therapeutic capable agent, and optionally the second compound. In an embodiment, the kit comprises the device and the second compound with or without the IFU for the second compound and/or a second compound device.

In one embodiment, the second compound may be a therapeutic capable agent, an optional another compound (e.g., the another therapeutic capable agent and/or the another enabling and/or enhancing compound), or a bio-active compound such as an anti-nausea drug; and being similar or different than that made available to the susceptible tissue site by the device; may be administered prior to, concurrent with, or subsequent to the implantig of the device (e.g., prosthesis) of the present invention. Examples of bio-active compounds include, but are not limited to, antiemetics such as ondansetron (e.g., ZOFRAN™), antiauseants such as dronabinol (e.g., MARINOL™) and ganisetron.Hcl (e.g., KYTRIL™).

The second compound may be administered from a pathway similar to or different than that used for the delivery of the therapeutic capable agent. By way of example, the second compound may be in the form of a tablet to be taken orally, a transdermal patch to be placed on the patient's skin, or administered subcutaneously, systemically by direct introduction to the blood stream, by way of inhalation, or through any other pathways and bodily orifices. Alternatively, the second compound may be made available to the intracorporeal body by a catheter. In an embodiment, the balloon of a balloon catheter (e.g., perfusion catheter), may be used to perfuse the second compound into the corporeal body or may be coated with the second compound. The second compound may be made available to the patient continuously or in discrete intervals, prior to, concurrent with, or subsequent to the interventional procedure.

The duration of the availability of the second compound usually may be shorter as compared to that of the therapeutic capable agent or optional another compound. In an embodiment, the second compound may be administered to the patient in a time period ranging from about 200 days prior to about 200 days after the interventional procedure, from about 30 days prior to about 30 days after the interventional procedure, from about 1 day prior to about 30 days after the interventional procedure, from about 200 days prior to about up to the interventional procedure, from about 3 months prior to about up to the interventional procedure, or from about 7 days to about 24 hours prior to the interventional procedure. The duration of the availability of the second compound as measured in the patient's blood may range from about 1 hour to about 120 days, from about 12 hours to about 60 days, or from about 24 hours to about 30 days.

In one embodiment, the second compound may be the same as the therapeutic capable agent of the device to provide a desired bullous level of the therapeutic capable agent in the corporeal body. The total amount made available to the susceptible tissue site from the second compound will typically be in a range from about 0.1 μg to about 10 mg, preferably in a range from about 10 μg to about 2 mg, more preferably in a range from about 50 μg to about 1.0 mg. In an embodiment the amount of the second compound administered to the patient on a single, acute dose or daily basis, ranges from about 0.5 mg to about 5 g, from about 1 mg to about 3 g, from about 2 g to about 3 g, from about 1 g to about 1.5 g. Examples of second compounds being provided at the latter series of doses include, mycophenolic acid, rapamycin, and their respective pro-drugs, metabolites, derivatives, and combinations thereof. In an example mycophenolic acid or rapamycin may be provided as a second compound at individual doses ranging from about 1 g to about 1.5 g, and from about 1 mg to about 3 mg, respectively; and at a daily dose ranging from about 2 g to about 3 g, and from about 2 mg to about 6 mg, respectively.

In operation, methods of delivering the therapeutic capable agents to the susceptible tissue site comprise positioning the source of the therapeutic capable agent within the intracorporeal site, such as the vascular lumen. The therapeutic capable agent is released and/or made available to the susceptible tissue site. In an embodiment, the releasing of the therapeutic capable agent occurs at a pre-determined time period following the positioning of the source. The delay in the release of the therapeutic capable agent may be for a sufficiently long period of time to allow sufficient generation of intimal tissue to reduce the occurrence of a thrombotic event. The device may comprise a rate-sustaining or rate-controlling element. In an embodiment, the source includes the rate-sustaining or rate-controlling element. In one embodiment, the releasing of the therapeutic capable agent may occur by surface degradation or hydrolysis of the source. In yet another embodiment, the release of the therapeutic capable agent may occur by bulk degradation of the source. In another embodiment, the releasing the therapeutic capable agent may occur by diffusion through the source. In an embodiment, a device including a source of therapeutic capable agent and incorporating any one or more features of the present invention is delivered to a corporeal site, such as an intracorporeal body (e.g., body lumen). The corporeal site may be a targeted corporeal site (such as a targeted intracorporeal site), which includes the susceptible tissue site, or a targeted site directly or indirectly providing the therapeutic capable agent to the susceptible tissue site. The therapeutic capable agent is made available to the susceptible tissue site, preferably, in a sustained or controlled manner over a period of time.

Methods of treatment generally include positioning the source including the at least one therapeutic capable agent and/or optional another compound within the intracorporeal body, concurrently with or subsequent to, an interventional treatment. More specifically, the therapeutic capable agent may be delivered to a targeted corporeal site (e.g., targeted intracorporeal site) which includes the susceptible tissue site or a targeted site providing the therapeutic capable agent to the susceptible tissue site, concurrently with or subsequent to the interventional treatment. By way of example, following the dilation of the stenotic region with a dilatation balloon, a device (such as a stent) according to the present invention, is delivered and implanted in the vessel. The therapeutic capable agent may be made available to the susceptible tissue site at amounts which may be sustainable, intermittent, or continuous; at one or more phases; and/or rates of delivery.

In an embodiment, the release of the therapeutic capable agent to the susceptible tissue site may be delayed. During the delay period none to small amounts of therapeutic capable agent may be released before the release of a substantial amount of therapeutic capable agent. Typically, the delay is sufficiently long to allow for sufficient generation of intimal tissue or cellularization at the treated site to reduce the occurrence of a thrombotic event.

In one embodiment, delay is sufficiently long to allow the generated neointima to cover at least partially the implanted expandable structure. In an embodiment, the therapeutic capable agent may be released in a time period, as measured from the time of implantig of the device, ranging from about 1 day to about 200 days; from about 1 day to about 45 days; or from about 7 days to about 21 days. In an embodiment, the method further includes directing energy at the device to effect release of the therapeutic capable agent from the device. The energy may include one or more of ultrasound, magnetic resonance imaging, magnetic field, radio frequency, temperature change, electromagnetic, x-ray, heat, vibration, gamma radiation, or microwave. In an embodiment, the therapeutic capable agent may be released at a total amount ranging from about 0.1 μg to about 10 g, from about 0.1 μg to about 10 mg, from about 1 μg to about 10 mg, from about 1 μg to about 2 mg, from about 10 μg to about 2 mg, or from about 50 μg to about 1 mg.

In another embodiment of a method of treatment, the releasing includes release of at least one optional another compound, as described above. The optional another compound may be another therapeutic capable agent or an enabling compound, as described above. The another compound may be released prior to, concurrent with, subsequent to the therapeutic capable agent, or sequentially with the therapeutic capable agent.

In an embodiment, a second compound, as described above, may be administered to the patient, prior to, concurrent with, or subsequent to the interventional procedure. The second compound may be administered from pathways, at time periods, and at levels, as described above.

In still another embodiment of the present invention, an improved method for delivering a therapeutic capable agent to an artery is provided. The method comprises implantig a prosthesis within the artery. The prosthesis releases the therapeutic capable agent. The prosthesis is configured to begin substantial release of the therapeutic capable agent after growth of at least one layer of cells over at least a part of the prosthesis.

Another method for luminal substance delivery comprises providing a luminal prosthesis comprising a matrix including the therapeutic capable agent and a matrix material formed from a rate-sustaining or rate-controlling element, as described above. In one embodiment, the matrix material undergoes degradation in a vascular environment. The degradation of the matrix material may take place over a predetermined time period with the substantial substance release beginning after substantial degradation of the matrix material.

### BRIEF DESCRIPTION OF THE DRAWINGS

FIGS. 1A through 1C are cross-sectional views of a device embodying features of the present invention and implanted in a body lumen.

FIGS. 2A through 2N are cross-sectional views of various embodiments of the delivery prosthesis of FIGS. 1A-1C taken along line 2-2.

FIG. 3 is a schematic representation of an exemplary stent for use as the device of the present invention.

FIG. 4 is a graphical representation of the release of a therapeutic capable agent over a predetermined time period.

FIG. 5 is a partial cross-sectional view of an embodiment of the prosthesis of FIGS. 1A-1C having a cellular growth thereon after being implanted.

FIGS. 6A through 61 illustrate features of an exemplary method for positioning the prosthesis of FIGS. 1A-1C in a blood vessel.

FIGS. 7A, 7B, 8A, 8B, 9A through 9E, 10A, 10B, 11A, and 11B are graphical representations of the performance of various therapeutic capable agents.

### DETAILED DESCRIPTION OF THE INVENTION

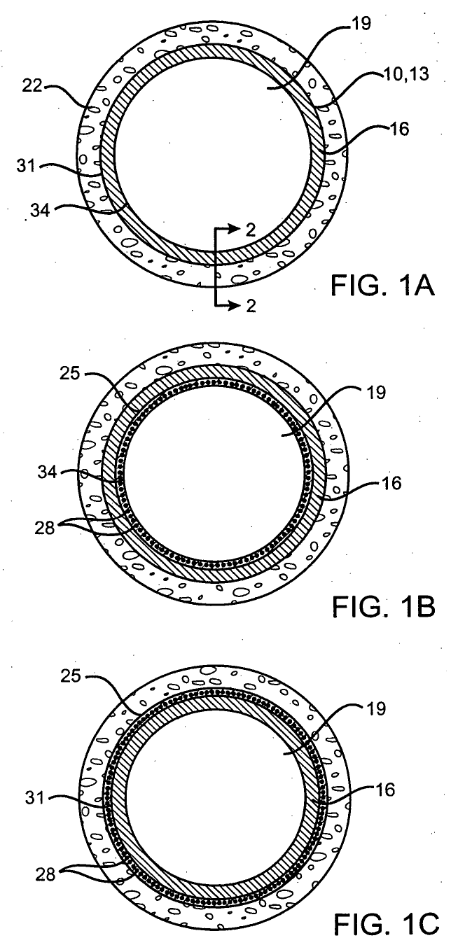

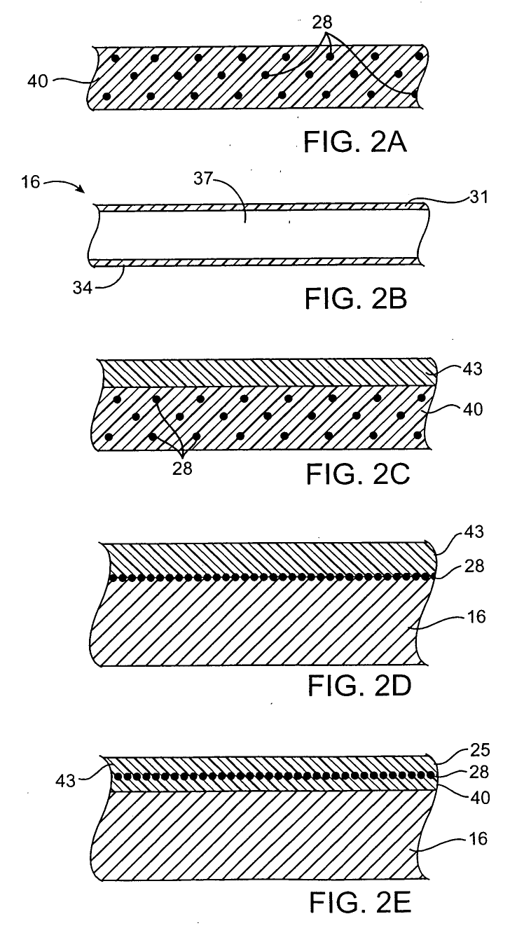

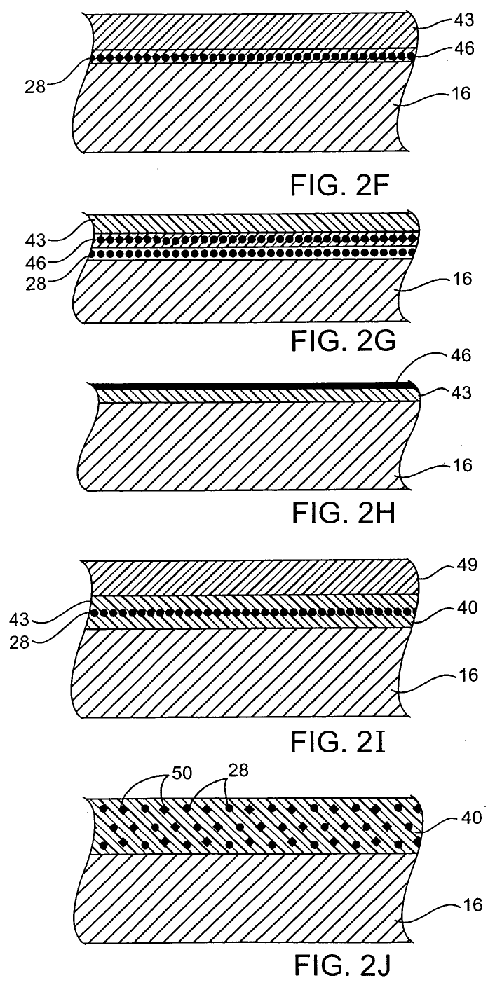

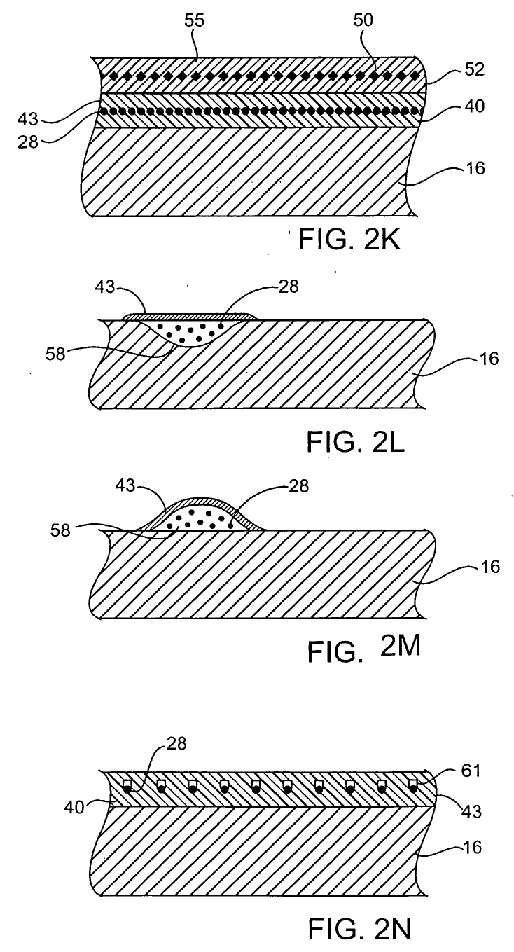

FIGS. 1A-1C, and cross-sectional drawings FIGS. 2A-2N, illustrate a device 10, such as a prosthesis 13, embodying features of the invention and generally including an expandable structure 16 implantable in an intracorporeal body, such as body lumen 19 including a susceptible tissue site 22, and a source 25 adjacent the expandable structure 16 including a therapeutic capable agent 28. The device 10, as shown, is disposed in the body lumen 19. It should be appreciated that although the source 25, as depicted in the figures, is disposed adjacent a surface of the expandable structure, the term “adjacent” is not intended to be limited by the exemplary figures or descriptions.

The expandable structure may be formed of any suitable material such as metals, polymers, or a combination thereof. In one embodiment, the expandable structure may be formed of an at least partially biodegradable material selected from the group consisting of polymeric material, metallic materials, or combinations thereof. The at least partially biodegradable material preferably degrades over time. Examples of polymeric material include poly-L-lactic acid, having a delayed degradation to allow for the recovery of the vessel before the structure is degraded. Examples of metallic material include metals or alloys degradable in the corporeal body, such as stainless steel. An exemplary stent for use in the present invention is described in co-pending U.S. patent application Ser. No. 09/565,560.

The therapeutic capable agent includes at least one compound, molecular species, and/or biologic agent that is either therapeutic as it is introduced to the subject under treatment, becomes therapeutic after entering being introduced to the subject under treatment as for example by way of reaction with a native or non-native substance or condition, or another introduced substance or condition. Examples of native conditions include pH (e.g., acidity), chemicals, temperature, salinity, osmolality, and conductivity; with non-native conditions including those such as magnetic fields, electromagnetic fields (such as radiofrequency and microwave), and ultrasound. In the present application, the “chemical name” of any of the therapeutic capable agents or other compounds is used to refer to the compound itself and to pro-drugs (precursor substances that are converted into an active form of the compound in the body), and/or pharmaceutical derivatives, analogues, or metabolites thereof (bio-active compound to which the compound converts within the body directly or upon introduction of other agents or conditions (e.g., enzymatic, chemical, energy), or environment (e.g., pH)).

The therapeutic capable agent may be selected from a group consisting of immunosuppressants, anti-inflammatories, anti-proliferatives, anti-migratory agents, anti-fibrotic agents, proapoptotics, vasodilators, calcium channel blockers, anti-neoplastics, anti-cancer agents, antibodies, anti-thrombotic agents, anti-platelet agents, IIb/IIIa agents, antiviral agents, MTOR (mammalian target of rapamycin) inhibitors, non-immunosuppressant agents, tyrosine kinase inhibitors, CDK inhibitors, bisphosphonates, NF-κB Decoy Oligo, proteins, oligomers, amino acids, peptides, genes, growth factors, anti-sense, metabolites, derivatives, agent incorporated in a vector such as a HVJ Envelop vector, and a combination thereof. Specific examples of therapeutic capable agent include: mycophenolic acid, mycophenolic acid derivatives (e.g., 2-methoxymethyl derivative and 2-methyl derivative), VX-148, VX-944, mycophenolate mofetil, mizoribine, methylprednisolone, dexamethasone, CERTICAN™ (e.g., everolimus, RAD), rapamycin, 32-deoxorapamycin (SAR943), ABT-578, ABT-773 (Abbot Labs), ABT-797 (Abbot Labs), TRIPTOLIDE™, METHOTREXATE™, phenylalkylamines (e.g., verapamil), benzothiazepines (e.g., diltiazem), 1,4-dihydropyridines (e.g., benidipine, nifedipine, nicarrdipine, isradipine, felodipine, amlodipine, nilvadipine, nisoldipine, manidipine, nitrendipine, barnidipine (HYPOCA™)), ASCOMYCIN™, PIMECROLIMUS™, WORTMANNIN™, LY294002, CAMPTOTHECIN™, silibinin, sylymarin, baicalein, histone deacetylase such as trichostatin A, PD-0183812, butyrolactone I, substituted purines (e.g., olomoucine, CGP74514, and its derivatives), polyhydroxylated flavones (e.g., flavopyridol), oxindole inhibitors (e.g., GW-8510, GW-2059, GW-5181), and indolinone derivatives (e.g., SU-5416), Zoledronic acid (i.e., ZOMETA™, Zoledronic acid, and (1-Hydroxy-2-imidazol-1-yl-phosphonoethyl)phosphonic acid monohydrate), isoquinoline, HA-1077 (1-(5-isoquinolinesulfonyl)-homopiperazine hydrochloride), TAS-301 (3-bis(4-methoxyphenyl)methylene-2-indolinone), TOPOTECAN™, hydroxyurea, TACROLIMUS™ (FK 506), cyclophosphamide, cyclosporine, daclizumab, azathioprine, prednisone, diferuloymethane, diferuloylmethane, diferulylmethane, GEMCITABINE™, cilostazol (PLETAL™), TRANILAST™, enalapril, quercetin, suramin, estradiol, cycloheximide, tiazofurin, zafurin, AP23573, rapamycin derivatives, non-immunosuppressive analogues of rapamycin (e.g rapalog, SAR943 (32-deoxorapamycin), AP21967, derivatives of rapalog, SAR943 (32-deoxorapamycin)), CCI-779 (an analogue of rapamcin available from Wyeth), sodium mycophenolic acid, benidipine hydrochloride, sirolimus, rapamune, phenylaminopyrimidine (or phenylpyrimidine-amine) derivatives (e.g., Imatinib (GLIVEC™)), other tyrosine inhibitors such as 4-[6-methoxy-7-(3-piperidine-1-yl-propoxy)-quinazolin-4-yl]-piperazine-1-carboxylicacid(4-isopropoxyphenyl)amide (CT53518 or MLN518 from Millennium Pharmaceutical), 5-Chloro-3-[(3,5-dimethylpyrrol-2-yl)methylene]-2-indolinone (SU6656), 5-Chloro-3-[(3,5-dimethylpyrrol-2-yl)methylene]-2-indolinone (SU5614 from Sugen), a water-soluble N,N-dimethylgly-cine ester prodrug CEP7055 that converts to CEP5214 in vivo from Cephalon, West Chester Pa., 4-Amino-5-(4-chlorophenyl)-7-(t-butyl)pyrazolo[3,4-d]pyrimidine (PP2 or AG1879), 6,7-Dimethyl-2-phenylquinoxaline (AG1295), TAUTOMYCIN™, Radicicol, Damnacanthal, Herbimycin A, 6-(2,6-dichloro-phenyl)-8-methyl-2-(3-methylsulfanyl-phenylamino)-8h-pyrido(2,3-d)pyrimidin-7-one (PD173955 from Parke-Davis), PD166326, PD183805, 4-[(3-Bromophenyl)amino]-6-propionylamidoquinazoline (PD 174265), 5-Chloro-3-[(3,5-dimethylpyrrol-2-yl)methylene]-2-indolinone (PD 153035), 4-[(3-Bromophenyl)amino]-6-acrylamidoquinazoline (PD168393), TARCEVA™ (erlotinib HCl), CI-1033, AEE788, CP-724,714 (from OSI Pharmaceutical), Geldanamycin, 17-(allylamino)-17-demethoxygeldanamycin (17-AG or 12-AAG), TARCEVA™, IRESSA™, and ZD4910, EGFR/ErbB2 inhibitor (CI1033; EKB569; GW2016; PK1166), VEGF receptor inhibitors (ZK222584;ZD6474), VEGFR/FGFR/PDGFR inhibitors (SU6668; SU11248; PTK787), NGF receptor (CEP2583), anti-EGF receptor MAbs (MAb225/ERBITUX™), anti-ErbB2 MAbs (MAb4D5/HERCEPTIN™), AVASTIN™, an anti-VEGF MAb, NF-κB Decoy Oligo, albumin, TSC1, TSC2, hamartin KIAA0243, VEGF, EGF, PDGF, FGF, Antisense phosphorothioate oligodeoxynucleotide (ODN), Anti-MTOR, Anti-p27 Anti-p53, Anti-Cdk, metabolites, derivatives, agent incorporated in a vector such as a HVJ Envelop vector, and/or combinations thereof.

Mycophenolic acid is an immunosuppressive drug produced by the fermentation of several penicillium brevi-compactum and related species (The Merck Index, Tenth Edition, 1983). It has a broad spectrum of activities, specific mode of action, and is tolerable in large doses with minimal side effects, Epinette et al., Journal of the American Academy of Dermatology, 17, pp. 962-971 (1987). Mycophenolic acid has been shown to have anti-tumor, anti-viral, anti-psoriatric, immunosuppressive, and anti-inflammatory activities, Lee et al., Pharmaceutical Research, 2, pp. 161-166 (1990), along with antibacterial and antifungal activities, Nelson et al., Journal of Medicinal Chemistry, 33, pp. 833-838 (1990). Of particular interest to the present invention, animal studies of accelerated arteriosclerosis have demonstrated that mycophenolic acid could also decrease the extent of smooth muscle cell proliferation, Gregory et al., Transplant Proc. 25, pp. 770 (1993).

Mycophenolic acid acts by inhibiting inosine monophosphate dehydrogenase and guanosine monophosphate synthetase enzymes in the de novo purine biosynthesis pathway. This may cause the cells to accumulate in the G1-S phase of the cell cycle and thus result in inhibition of DNA synthesis and cell proliferation (hyperplasia). In the present application, the term “mycophenolic acid” is used to refer to mycophenolic acid itself, pro-drugs (precursor substances that are converted into an active form of mycophenolic acid in the body), and/or pharmaceutical derivatives thereof, analogues thereof, or metabolites thereof (bio-active compound to which the mycophenolic acid converts within the body directly or upon introduction of other agents or conditions (e.g., enzymatic, chemical, energy)). For example, a pro-drug such as mycophenolate mofetil may be biotransformed or metabolically converted to a biologically active form of mycophenolic acid when administered in the body. A number of derivatives of mycophenolic acid are taught in U.S. Pat. Nos. 4,786,637, 4,753,935, 4,727,069, 4,686,234, 3,903,071, and 3,705,894, all incorporated herein by reference, as well as pharmaceutically acceptable salts thereof.

Mizoribine acts by inhibiting inosine monophosphate dehydrogenase and guanosine monophosphate synthetase enzymes in the de novo purine biosynthesis pathway. This may cause the cells to accumulate in the G1-S phase of the cell cycle and thus result in inhibition of DNA synthesis and cell proliferation (hyperplasia).

Methylprednisolone is a synthetic steroid in the class of glucocorticoids that suppresses acute and chronic inflammations. In addition, it reduced vascular smooth muscle generation. Its anti-inflammatory actions include inhibition of accumulation of inflammatory cells (including macrophages and leukocytes) at inflammation sites and inhibition of phagocytosis, lysosomal enzyme release, and synthesis and/or release of several chemical mediators. Its immunosuppressant actions may involve prevention/suppression of cell-mediated (delayed hypersensitivity) immune reactions and more specific actions affecting immune response. Immunosuppressant actions may also contribute significantly to the anti-inflammatory effect.

CERTICAN™, also known as everolimus, SDZ-RAD, RAD, RAD666, or 40-0-(2-hydroxy)ethyl-rapamycin, is a potent immunosuppressant and anti-inflammatory agent. In particular, CERTICAN™ acts to inhibit the activation and proliferation of T lymphocytes in response to stimulation by antigens, cytokines (IL-2, IL-4, and IL-15), and other growth-promoting lymphokines. CERTICAN™ also inhibits antibody production. In cells, CERTICAN™ binds to the immunophilin, FK Binding Protein-12 (FKBP-12). The Certican:FKBP-12 complex, which has no effect on calcineurin activity, binds to and inhibits the activation of the MTOR, a key regulatory kinase. This inhibition suppresses cytokine-driven T-cell proliferation, inhibiting the progression of the cell cycle from the G1 to the S phase, selectively blocking signals leading to the activation of p70s6k, p33cdk2 and p34cdc2. Thus, CERTICAN™ administration results in inhibiting proliferation of T and B cells, inflammatory cells, as well as smooth muscle cells (hyperplasia).

TRIPTOLIDE™ or related compounds, such as, tripdiolide, diterpenes, triterpenes, diterpene epoxides, diterpenoid epoxide, triepoxides, or tripterygium wifordii hook F (TWHF), are also potent immunosuppressant and anti-inflammatory agents. Specifically, TRIPTOLIDE™ has been shown to inhibit the expression of IL-2 in activated T cells at the level of purine-box/nuclear factor and NF-kappaB mediated transcription activation. TRIPTOLIDE™ may induce apoptosis in tumor cells and potentiate a tumor necrosis factor (TNF-alpFha) induction of apoptosis in part through the suppression of c-IAP2 and c-IAP1 induction. TRIPTOLIDE™ inhibits the transcriptional activation, but not the DNA binding, of nuclear factor-kappaB. TRIPTOLIDE™ may also inhibit expression of the PMA-induced genes tumor necrosis factor-alpha, IL-8, macrophage inflammatory protein-2alpha, intercellular adhesion molecule-1, integrin beta6, vascular endothelial growth factor, granulocyte macrophage colony-stimulating factor (GM-CSF), GATA-3, fra-1, and NF45. TRIPTOLIDE™ inhibits constitutively expressed cell cycle regulators and survival genes, such as, cyclins D1, B1, A1, cdc-25, bcl-x, and c-jun. Thus anti-inflammatory, antiproliferative, and proapoptotic properties of TRIPTOLIDE™ are associated with inhibition of nuclear factor-kappaB signaling and inhibition of the genes known to regulate cell cycle progression and survival. TRIPTOLIDE™ inhibits mRNA expression of c-myc and PDGF in vascular smooth muscle cells, hence resulting in the inhibition of proliferative smooth muscle cells (hyperplasia).

METHOTREXATE™, formerly amethopterin, is an immunosuppressant and anti-proliferative agent that has been used in the treatment of certain neoplastic diseases and severe psoriasis. Chemically METHOTREXATE™ is N-[4[[(2,4-diamino-6-pteridinyl)methyl]methylamino]benzoyl]-L-glutamic acid. In particular, METHOTREXATE™ inhibits dihydrofolic acid reductase, thereby inhibiting the reduction of dihydrofolates to tetrahydrofolates in the process of DNA synthesis, repair, and cellular replication. Actively proliferating tissues such as malignant cells, bone marrow, fetal cells, buccal and intestinal mucosa, and cells of the urinary bladder are in general more sensitive to this METHOTREXATE™ effect. When cellular proliferation in malignant tissue is greater than in most normal tissues, METHOTREXATE™ may impair malignant growth without irreversible damage to normal tissues. Approximately 50% of the drug may be reversibly bound to serum proteins. After absorption, METHOTREXATE™ undergoes hepatic and intracellular metabolism to polyglutamated forms which can be converted back to METHOTREXATE™ by hydrolase enzymes. These polyglutamates act as inhibitors of dihydrofolate reductase and thymidine synthetase.

Calcium channel blockers are commonly used as anti-hypertensive agents that relax vascular smooth muscle and reduce vascular resistance. They do this by inhibiting the movement and binding of calcium ions, which play an integral role in regulating skeletal and smooth muscle contractility and in the performance of the normal and diseased heart. Two types of calcium channel blockers are used in clinical situations: those that are selective for L-type (long-lasting, large-current, or slow), voltage-dependent calcium channels, and those that are nonselective. In clinical practice, selective agents are primarily used.

Often considered a homogeneous family of drugs, selective calcium channel blockers actually have marked individual differences in chemical structure, binding site, tissue selectivity, and, consequently, clinical activity and therapeutic indications. These agents can be grouped into three discrete chemical classes: the phenylalkylamines (e.g., verapamil), the benzothiazepines (e.g., diltiazem), and the 1,4-dihydropyridines (e.g., benidipine, nifedipine, nicarrdipine, isradipine, felodipine, amlodipine, nilvadipine, nisoldipine, manidipine, nitrendipine). Verapamil and diltiazem are pharmacologically more similar to each other than either is to the dihydropyridines, which has prompted some to recommend delineating verapamil and diltiazem (non-dihydropyridines) as one subgroup of calcium channel blockers and the dihydropyridines as another.

Although all three types of selective calcium channel blockers interact with the alpha1 subunit of the L-type calcium channel, each binds to a different receptor site. A complex allosteric relationship exists among these receptor sites. For example, drugs binding at the dihydropyridine site appear to increase the affinity of diltiazem for the benzothiazepine site, and vice versa. In contrast, the binding of verapamil at the phenylalkylamine site appears to reduce the affinities of diltiazem and the dihydropyridine calcium channel blockers for binding at their respective sites.

The binding sites for all three chemical types of calcium channel blocker are present in many tissues, including myocardium, smooth muscle, skeletal muscle, and glandular tissue. However, the activity of each calcium channel blocker in a particular tissue varies. Nifedipine and other dihydropyridines act preferentially on vascular smooth muscle, exerting potent peripheral vasodilating effects. Verapamil and diltiazem are less specific for peripheral vascular smooth muscle and more active in the myocardium and cardiac conductive tissues.

All the selective calcium channel blockers are well absorbed after oral administration, although there are marked differences in oral bioavailability that relate to differences in first-pass metabolism. Verapamil and isradipine undergo fairly extensive first-pass metabolism, whereas diltiazem, nifedipine, and nicardipine do not. Protein binding percentages are higher with the dihydropyridines than with either diltiazem or verapamil. With nifedipine and possibly other dihydropyridines, protein binding is concentration dependent, allowing for the possibility of protein-binding interactions, although none of clinical significance has been reported. With verapamil and diltiazem, protein binding is independent of drug concentrations, making displacement interactions unlikely.

Benidipine—Benidipine hydrochloride, ((±)-(R*)-3-[(R*)-1-benzyl-3-piperidyl]methyl 1,4-dihydro-2,6-dimethyl-4-(m-nitrophenyl)-3,5-pyridine dicarboxylate hydrochloride), is a long-acting, L-type Ca²⁺ channel blocker. Ca²⁺ channel blockers are widely used for the treatment of ischemic heart disease and systemic hypertension because of their ability to effectively dilate coronary and systemic arteries. Ca²⁺ channel blockers increase coronary blood flow (CBF) in inhibiting Ca²⁺ entry into smooth muscle cells. Since Ca²⁺ overload is deleterious for the maintenance of cellular homeostasis, Ca²⁺ channel blockers are believed to be effective in attenuating Ca²⁺ overload. Because it blocks Ca²⁺ entry, it inhibits the proliferation of smooth muscle cell.

Benidipine can protect endothelial cell function in the renal resistance arteries of hypertensive rats and the mesenteric arteries of rats subjected to circulatory shock. Endothelial cell function is important for the preservation of organ function during ischemic or hypertensive stress. Benidipine has a cardioprotective effect during myocardial ischemia and reperfusion injury. Since myocardial ischemia impairs endothelial cell function by the activation of platelets and leukocytes, benidipine may attenuate endothelial cell dysfunction and increase the production of nitric oxide in ischemic hearts.

ASCOMYCIN™ (molecular formula: C₄₃H₆₉NO₁₂; molecular weight: 792.02; CAS No. 104987-12-4) has produced significant anti-inflammatory and immunosuppressant activity. ASCOMYCIN™ and its derivatives (i.e., PIMECROLIMUS™) has been shown to selectively inhibit inflammatory cytokine release. The drug binds to the cytosolic immunophilin receptor macrophilin-12, and the resulting complex inhibits the phosphatase calcineurin, thus blocking T-cell activation and cytokine release. It inhibits production of Th1 cytokines (interleukin-2 and interferon-gamma) and Th2 cytokines (interleukin-10 and interleukin-4). ASCOMYCIN™ and its derivatives has also been demonstrated to similarly inhibit mast cell. It is a strong immunosuppressant and inhibits allogenic T-lymphocyte proliferation. It binds with high affinity to FKBP and inhibits calcineurin phosphatase in the nM range.

ASCOMYCIN™ and its derivatives affect calcineurin-mediated signal transduction. It is a natural product of bacteria and fungi, respectively, with potent immunosuppressive, anti-inflammatory, and antimicrobial activity. Despite differing chemical structures, ASCOMYCIN™ is a macrolide where its mechanisms of action and cellular effects result in the inhibition of the protein phosphatase calcineurin. This drug is hydrophobic and thought to diffuse across the plasma membrane. Once inside the cell, ASCOMYCIN™ forms complexes with their major receptors, FKBP12. FKBP12 are small, ubiquitous, cytosolic proteins that catalyse cis-trans prolyl isomerization, a reaction that can be a rate-limiting step in protein folding. Binding of ASCOMYCIN™ to FKBP 12 inhibits prolyl-isomerase activity. However, this inhibition is not the major toxic effect in the cell. Instead, the FKBP12-ascomycin complex binds to and inhibits calcineurin (a serine-threonine-specific protein phosphatase), which is activated by calmodulin in response to intracellular calcium-ion increases. The molecular nature of this interaction is now known in considerable detail, as the structures of both calcineurin alone and in a ternary complex with FKBP12-ascomycin have both been solved at high resolution.

WORTMANNIN™ (CAS No. 19545-26-7, synonym SL-2052, molecular formula: C23H24O8 formula weight: 428.4 (anhydrous)) has significant anti-inflammatory and immunosuppressant activity. WORTMANNIN™, a fungal metabolite, is a specific and potent inhibitor of myosin light chain kinase and a potent inhibitor of neutrophil activation by inhibiting F-met-leu(FMLP)-phe-stimulated superoxide anion production without affecting intracellular calcium mobilization. It inhibits FMLP-stimulated phospholipase D activation without direct inhibition of the enzyme. It also inhibits phosphatidylinositol-3-kinase (PI3-kinase) and blocks IgE-mediated histamine release in rat basophilic leukemia cells and human basophils.

WORTMANNIN™ is a potent and specific inhibitor of phosphatidylinositol 3-kinase (PI3-K) with an IC₅₀ of 2-4 nM. It also inhibits myosin light chain kinase at a 100-fold higher concentration. Inhibition of PI3-K/Akt signal transduction cascade enhances the apoptotic effects of radiation or serum withdrawal and blocks the antiapoptotic effect of cytokines. Inhibition of PI3-K by WORTMANNIN™ also blocks many of the short-term metabolic effects induced by insulin receptor activation.

Phosphatidylinositol-3-kinase participates in the signal transduction pathway responsible for histamine secretion following stimulation of high affinity immunoglobulin E receptor (FceRI). WORTMANNIN™ blocks these responses through direct interaction with the catalytic subunits (110 kDa) of PI3-kinase enzyme. WORTMANNIN™ inhibited the activity of partially purified PI3-kinase from calf thymus at concentrations as low as 1.0 nM and with IC50 values of 3.0 nM. Inhibition was irreversible. WORTMANNIN™ inhibited both FceRI-mediated histamine secretion and leukotriene release up to 80% with IC50 values of 2.0 and 3.0 nM, respectively. Additional functions of WORTMANNIN™ include immunosuppressive activity, strong anti-inflammatory activity, and suppression of cellular responses such as respiratory burst and exocytosis in neutrophils and catecholamine release in adrenal chromaffin cells. Aggregation and serotonin release in platelets were reported using a final concentration of 1 M of WORTMANNIN™ in 0.01% DMSO.

WORTMANNIN™ is a hydrophobic steroid-related product of the fungus Talaromyces wortmanni that inhibits signal-transduction pathways. For example, WORTMANNIN™ inhibits stimulation of neutrophils, histamine secretion by basophilic leukaemia cells, and nitric-oxide production in chicken macrophages. In mammalian cells, several lines of evidence indicate that the growth-factor-activated PI-3 kinase is potently inhibited by WORTMANNIN™. First, WORTMANNIN™ blocks the antigen-dependent stimulation of PI-3-kinase activity in basophils 54 and the insulin-stimulated PI-3-kinase activity in adipocytes. WORTMANNIN™ also inhibits stimulated PIns-(3,4,5)P 3 production in neutrophils, consistent with a block in PIns-(4,5)P phosphorylation by PI-3 kinase. Purified p110-p85 PI-3 kinase is potently inhibited by WORTMANNIN™ in vitro. Finally, studies with anti-WORTMANNIN™ antibodies and site-directed mutagenesis reveal that WORTMANNIN™ forms a covalent complex with an active-site residue of bovine PI-3 kinase, lysine 802 of the 110 kDa catalytic subunit. This active-site lysine residue is essential for PI-3 kinase activity and is well conserved throughout all members of the PI-kinase-related protein family.

LY294002 has produced significant anti-inflammatory and immunosuppressant activity. LY294002 has been used in some cases to confirm the effects of WORTMANNIN™ attributed to inhibition of PI-3 kinase, but this compound also inhibits MTOR and may inhibit other WORTMANNIN™ targets as well. Hence, more enzyme-specific analogues of WORTMANNIN™ would be valuable reagents to probe the intracellular functions of this intriguing family of enzymes. The WORTMANNIN™ analogue demethoxyviridin has been shown to inhibit an as-yet-unidentified PI-4-kinase activity in Schizosaccharomyces pombe that is much less sensitive to WORTMANNIN™, indicating that analogues with greater specificity may be obtained.

CAMPTOTHECIN™ and TOPOTECAN™ (hycamtin)—CAMPTOTHECIN™ (molecular formula: C₂₀H₁₆N₂O₄, molecular weight: 348.4, CAS No. 7689-03-4) and its analogues, including TOPOTECAN™ (9-Dimethylaminomethyl-10-hydroxycamptothecin, HCl salt 1H-Pyrano[3′,4′:6,7]indolizino[1,2-b]quinoline-3,14(4H,12H)-dione, 4-ethyl-4,9-dihydroxy-10-[(dimethylamino)methyl]-,HCl salt (S) molecular formula: C₂₃H₂₃N₃O₅·HCl, molecular weight: 457.9), are anti-neoplastic agents, believed to exert cytotoxic effects through the inhibition of topoisomerase I. This is the only known class of drug that exhibits this mechanism of action. However, inhibition of topoisomerase activity is not an unknown mechanism of action since many classes of drugs (e.g., epipodophyllotoxins) operate through inhibition of topoisomerase II (topo II).

Topoisomerases are enzymes which break strands of DNA so that the strands can be rotated around each other and then the break resealed. They can be divided into two classes according to the nature of the mechanisms of action they employ.

One of the most promising new drug classes includes the topoisomerase I inhibitors. This class is structurally related to the natural compound CAMPTOTHECIN™, which is derived from the Chinese Camptotheca acuminata plant. Topoisomerase I inhibitors differ from topoisomerase II inhibitors, such as etoposide, in that they bind to the topoisomerase-DNA complex. Cell death ensues when the DNA helix cannot rebuild after uncoiling. The two most promising compounds in this class are irinotecan and TOPOTECAN™. In Phase II trials, they have shown activity against a variety of cancers, including colorectal cancer. The success of TOPOTECAN™ in patients with previously treated small-cell lung cancer (response rate as high as 39 percent) and ovarian cancer (response rate as high as 61 percent) has increased interest in Phase III trials with this drug.

Type I topoisomerase (topo I) is a monomeric protein of about 100 Kilodaltons (KDa). It is capable of making a transient break in a single strand of the DNA helix. This reduces the torsional strain on the DNA and allows the DNA to unwind ahead of the replication fork. This enzyme is capable of relaxing highly negatively supercoiled DNA. In the eukaryotic version of this enzyme, a phosphotyrosyl bond is formed between the enzyme and the 3′ end of the DNA break. In this process there is a transfer of a phosphodiester bond in the DNA to the protein. The structure of the DNA is manipulated and the DNA is rejoined. Since the reaction requires only the transfer of bonds, not irreversible hydrolysis, no input of energy is required. Topo I is believed to function in DNA replication, RNA transcription, genetic recombination, chromosomal condensation/decondensation, and in viral encapsulation. Its presence is not cell-cycle dependent and it is found in quiescent as well as proliferating cells. It appears, however, that this enzyme is not required for the viability of cells. Topo II seems to fulfill the functions of topo I when it is absent. Double mutants, which lack both topo I and II have defects of replication and transcription.

Cells lacking the topo I enzyme are resistant to CAMPTOTHECIN™, while cells containing higher topo I levels are hypersensitive to these drugs. The CAMPTOTHECIN™ appear to block the rejoining step of the breakage-reunion reaction of the enzyme, leaving the enzyme covalently bound to DNA. This results in protein associated single strand breaks in the DNA.

TOPOTECAN™ has demonstrated good antitumor activity (increased life spans (ILS)>95% s) in several intraperitoneally (IP) and intravenously (IV) implanted murine tumor systems, including P388 leukemia, L1210 leukemia, B16 melanoma, Lewis lung carcinoma, and M5076 reticulum cell sarcoma. TOPOTECAN™ was equally effective when administered IP or IV against IP or IV implanted tumors. Subcutaneous administration did not result in any local tissue damage. This drug was also equally effective when administered enterally or parenterally in some tumors, suggesting that, in mice, the bioavailability is high.

The antitumor activity of TOPOTECAN™ in tumor-bearing mice can be enhanced by using an intermittent dosing regimen. Results were dependent upon how sensitive the tumor model was to bolus treatment with TOPOTECAN™. In studies in which TOPOTECAN™ was administered every three hours for 4 doses, a broader therapeutic dose range was noted in tumors that were quite sensitive to bolus therapy, including IV-implanted L1210 leukemia, IP M5076 reticulum sarcoma, SC colon 51, and SC B16 melanoma. In tumor types that were less sensitive to bolus therapy, such as SC implanted colon 26 and Madison 109 lung carcinomas, the divided dose resulted in a greater degree of inhibition at the MTD.

The activity of TOPOTECAN™ has also been investigated using a human tumor clonogenic assay. Fifty-five human tumor specimens were exposed to TOPOTECAN™ for one hour at a concentration of either 1 of 10 μg/ml or as a continuous exposure (0.1 or 1.0 μg/ml). At a concentration of 0.1 μg/ml of continuous exposure, response rates of 29, 27, and 37% were seen against breast, non-small cell lung, and ovarian cancers, respectively. Activity was also seen against stomach, colon, and renal cancer, and mesothelioma. Incomplete cross-resistance was noted with doxorubicin, 5-FU, and cyclophosphamide.

Hydroxyurea (Hydrea)—Hydroxyurea (molecular formula: CH₄N₂O₂, molecular weight: 76.06, CAS No. 127-07-1) is an anti-neoplastic agent. It is readily available drug that has been in use for three decades in treating certain kinds of leukemia and other cancers. It may also be promising for treatment of sickle cell disease. The exact mechanism of action has been unknown. It has been known that hydroxyurea immediately inhibits DNA synthesis without inhibiting the synthesis of RNA or protein, but until recently it was not known how it did this.

GEMCITABINE™ (Gemzar) (Gemcitabine hydrochloride; 2′-deoxy-2′,2′-difluorocytidine) is an anti-neoplastic agent. GEMCITABINE™ induces programmed cell death and activates protein kinase C in BG-1 human ovarian cancer cells. It is a known antitumor nucleoside where the mechanism of action of GEMCITABINE™ is via inhibition of DNA and RNA synthesis.

GEMCITABINE™ is a novel deoxycytidine analogue, a pyrimidine antimetabolite related to cytarabine, which was originally investigated for its antiviral effects but has since been developed as an anti-cancer therapy. GEMCITABINE™ exhibits cell phase specificity, primarily killing cells undergoing DNA synthesis (S-phase) and also blocking the progression of cells through the G1/S-phase boundary. GEMCITABINE™ is a pro-drug and is metabolized intracellularly to the active diphosphate (dFdCDP) and triphosphate (dFdCTP) nucleosides. The cytotoxic effects of GEMCITABINE™ are exerted through dFdCDP-assisted incorporation of dFdCTP into DNA, resulting in inhibition of DNA synthesis and induction of apoptosis.

GEMCITABINE™ exhibits significant cytotoxicity activity against a variety of cultured murine and human tumor cells. It exhibits cell phase specificity, primarily killing cells undergoing DNA synthesis (S-phase) and under certain conditions blocking the progression of cells through the G1/S-phase boundary. In vitro, the cytotoxic action of GEMCITABINE™ is both concentration and time dependant.

In animal tumor models, the antitumor activity of GEMCITABINE™ is schedule dependant. When administered daily, GEMCITABINE™ causes death in animals with minimal anti-tumor activity. However when every 3rd or 4th day dosing schedule is used, GEMCITABINE™ can be given at non-lethal doses that have excellent anti-tumor activity against a broad range of mouse tumors.

Rapamycin derivatives and MTOR (mammalian target of rapamycin) inhibitors such as AP23573 (sold commercially by Ariad Pharmaceuticals, Cambridge Mass.) inhibit the activity of MTOR and disrupt key signal transduction pathways, including those regulated by the p70s6 and PHAS-I kinases, resulting in cell cycle arrest at the G1-S boundary. These inhibitors bind with high affinity to FKBP and then to the large P13K homolog FRAP (RAFT, MTOR). Examples of rapamycin derivatives include fluorinated esters of rapamycin, amide esters of rapamycin, carbamates of rapamycin, sulyl ethers of rapamycin, 27-hydroxyrapamycin, O-arylrapamycin, O-alkylrapamycin, O-alkenylrapamycin, O-alkynlrapamycin, rapamycin arylcarbonyl carbamates, rapamycin alkoxycarbonyl carbamates, O-heteroarylrapamycin, O-alkylheteroarylrapamycin, O-alkenylheteroarylrapamycin, O-alkynlheteroarylrapamycin, imidazolidylrapamycin, 32-deoxorapamycin, deuterated rapamycin (from Isotechnika, Canada), and the like.

In some instances, when the patient is immuno-compromised due to a disease such as AIDS, the use of AP23573, rapamycin, SAR943 (32-deoxorapamycin), or rapamycin derivatives may be compromised by its cell cycle inhibitory effects (the result of inhibiting FRAP kinase activity, which in T cells leads to immunosuppression). To overcome this limitation, non-immunosuppressant agents may be used, such as non-immunosuppressive analogues of rapamycin (e.g., rapalog (AP21967 sold commercially by Ariad Pharmaceuticals, Cambridge Mass.) or derivatives of rapalog (sold commercially by Ariad Pharmaceuticals, Cambridge Mass.)), which have been chemically modified so that they no longer bind to FRAP/MTOR and greatly reduce immunosuppressive activity.

Tyrosine Kinase Inhibitors, such as TCA or phenylaminopyrimidine (or phenylpyrimidine-amine) derivatives (e.g., Imatinib (GLIVEC™)), may also inhibit restenosis. Inhibitors of protein kinase C (PKC), such as phenylaminopyrimidine (or phenylpyrimidine-amine) derivatives, provide strong PKC inhibition, particularly with derivatives bearing a 3′-pyridyl group at the 3′-position of the pyrimidine. Also, the presence of an amide group on the phenyl ring may provide inhibitory activity against tyrosine kinases, such as the BCR-ABL kinase. Attachments of a highly polar side chain of N-methylpiperazine may improve solubility and oral bioavailability. It is believed that this moiety may bind with protein kinases through hydrogen bonding and increase inhibitory effects. Phenylaminopyrimidine (or phenylpyrimidine-amine) derivatives, such as Imatinib (GLIVEC™), may inhibit the autophosphorylation of essentially three kinases: BCR-ABL; cKIT; and the platelet derived growth factor (PDGF) receptor. The TCA has been shown in cell culture and animal models to inhibit tumor growth. It may also cause selective apoptosis.

Different types of phenylpyrimidine-amines have been described that have one or more inhibitory effects (BCR-ABL; cKIT; PDFG receptor inhibitor).

Examples of suitable N-phenyl-2-pyrimidine-amine type inhibitors include compounds of Formulas I through XI, as provided below: 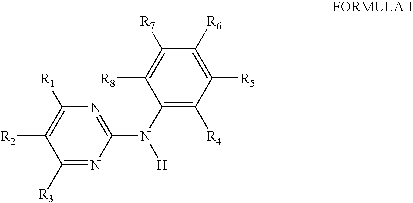
wherein R₁ is 4-pyrazinyl, 1-methyl-1H-pyrrolyl, amino- or amino-lower alkyl-substituted phenyl, wherein the amino group in each case is free, alkylated or acylated, 1H-indolyl or 1H-imidazolyl bonded at a five-membered ring carbon atom, or unsubstituted or lower alkyl-substituted pyridyl bonded at a ring carbon atom and unsubstituted or substituted at the nitrogen atom by oxygen; R₂ and R₃ are each independent of one another hydrogen or lower alkyl; one or two of the radicals R₄, R₅, R₆, R₇ and R₈ are each nitro, fluoro-substituted lower alkoxy or a radical of following formula:
   —N(R₉)—C(═X)—(Y)n—R₁₀
wherein R₉ is hydrogen or lower alkyl, X is oxo, thio, imino, N-lower alkyl-imino, hydroximino or O-lower alkyl-hydroximino, Y is oxygen or the group NH, n is 0 or 1 and R₁₀ is an aliphatic radical having at least 5 carbon atoms, or an aromatic, aromatic-aliphatic, cycloaliphatic, cycloaliphatic-aliphatic, heterocyclic or hetero-cyclicaliphatic radical, and the remaining radicals of the group R₄, R₅, R₆, R₇ and R₈ are each independent of the one another hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy, or a salt of such a compound having at least one salt-forming group. 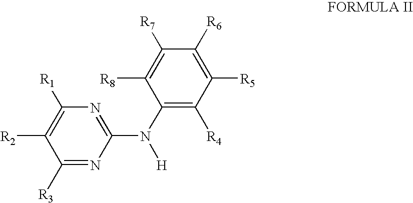
wherein one or two of the radicals R₄, R₅, R₆, R₇ and R₈ are each nitro or a radical of formula:
   —N(R₉)—C(═X)—(Y)ₙ—R₁₀
wherein R₉ is hydrogen or lower alkyl, X is oxo, thio, imino, N-lower alkyl-imino, hydroximino or O-lower alkyl-hydroximino, Y is oxygen or the group NH, n is 0 or 1 and R₁₀ is an aliphatic radical having at least 5 carbon atoms or an aromatic, aromatic-aliphatic, cycloaliphatic, cycloaliphatic-aliphatic, heterocyclic or hetero-cyclicaliphatic radical, and the remaining radicals of the group R₄, R₅, R₆, R₇ and R₈ are each independent of the one another hydrogen, lower alkyl that is unsubstituted or substituted by free or alkylated amino, piperazinyl, piperidinyl, pyrrolidinyl or by morpholinyl, or lower alkanoyl, trifluoromethyl, free, etherified or esterifed hydroxy, free, alkylated or acylated amino or free or esterified carboxy, and the remaining substituents are as defined as above or a salt of such a compound having at least one salt-forming group. 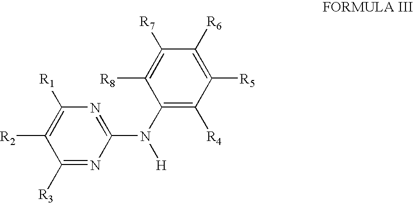
wherein R₁ is 4-pyrazinyl, 1-methyl-1H-pyrrolyl, amino- or amino-lower alkyl-substituted phenyl, wherein the amino group in each case is free, alkylated by one or two lower alkyl radicals or acylated by lower alkanoyl or by benzoyl, 1H-indolyl or 1H-imidazolyl bonded at a five-membered ring carbon atom, or unsubstituted or lower alkyl-substituted pyridyl bonded at a ring carbon atom and unsubstituted or substituted at the nitrogen atom by oxygen; R₂ and R₃ are each independent of one another hydrogen or lower alkyl, one or two of the radicals R₄, R₅, R₆, R₇ and R₈ are each nitro, fluoro-substituted lower alkoxy or a radical of formula:
   —N(R₉)—C(═X)—(Y)n—R₁₀
wherein R₉ is hydrogen or lower alkyl, X is oxo, thio, imino, N-lower alkyl-imino, hydroximino or O-lower alkyl-hydroximino, Y is oxygen or the group NH, n is 0 or 1 and R₁₀ is an aliphatic hydrocarbon radical having 5-22 carbon atoms, a phenyl or naphthyl radical each of which is unsubstituted or substituted by cyano, lower alkyl, hydroxy-lower alkyl, amino-lower alkyl, (4-methyl-piperazinyl)-lower alkyl, trifluoromethyl, hydroxy, lower alkoxy, lower alkanoyloxy, halogen, amino, lower alkylamino, di-lower alkylamino, lower alkanoylamino, benzoylamino, carboxy or by lower alkoxycarbonyl, or phenyl-lower alkyl wherein the phenyl radical is unsubstituted or substituted as indicated above, a cycloalkyl or cycloalkenyl radical having up to 30 carbon atoms, cycloalkyl-lower alkyl or cycloalkenyl-lower alkyl each having up to 30 carbon atoms in the cycloalkyl or cycloalkenyl moiety, a monocyclic radical having 5 or 6 ring members and 1-3 ring hetero atoms selected from nitrogen, oxygen and sulfur, to which radical one or two benzene radicals may be fused, or lower alkyl substituted by such a monocyclic radical, and the remaining radicals from the group R₄, R₅, R₆, R₇ and R₈ are each independent of one another hydrogen, lower alkyl that is unsubstituted or substituted by amino, lower alkylamino, di-lower alkylamino, piperazinyl, piperidinyl, pyrrolidinyl or by morpholinyl, or lower alkanoyl, trifluoromethyl, hydroxy, lower alkoxy, lower alkanoyloxy, halogen, amino, lower alkylamino, di-lower alkylamino, lower alkanoylamino, benzoylamino, carboxy or lower alkoxycarbonyl, or a salt of such a compound having at least one salt-forming group. R₄ and R₈ can be hydrogen and the remaining substituents are as defined in above, or a pharmaceutically acceptable salt of such a compound having at least one salt-forming group. 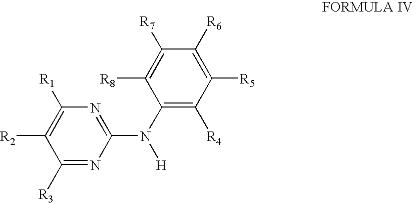
wherein R₁ is pyridyl bonded at a ring carbon atom and unsubstituted or substituted at the nitrogen atom by oxygen; R₂ and R₃ are each hydrogen; R₄ is hydrogen or lower alkyl; R₅ is hydrogen, lower alkyl or fluoro-substituted lower alkoxy; R₆ is hydrogen; R₇ is nitro, fluoro-substituted lower alkoxy or a radical of formula:
   —N(R₉)—C(═X)—(Y)n—R₁₀
wherein R₉ is hydrogen, X is oxo, Y is oxygen or the group NH, n is 0 and R₁₀ is an aliphatic hydrocarbon radical having 5-22 carbon atoms, a phenyl radical that is unsubstituted or substituted by cyano, lower alkyl, (4-methyl-piperazinyl)-lower alkyl, lower alkoxy, halogen or by carboxy; a cycloalkyl radical having up to 30 carbon atoms or a monocyclic radical having 5 or 6 ring members and 1-3 sulfur ring atoms, and R₈ is hydrogen, or a pharmaceutically acceptable salt of such a compound having at least one salt-forming group. R₄ and R₈ can be hydrogen and the remaining substituents are as defined above, or a pharmaceutically acceptable salt of such a compound having at least one salt-forming group. 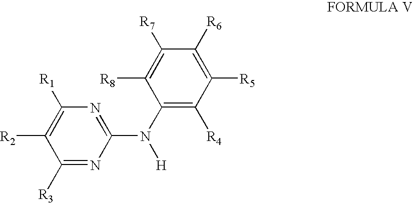
wherein R₁ is pyridyl or N-oxido-pyridyl each of which is bonded at a carbon atom; R₂ and R₃ are each hydrogen; R₄ is hydrogen or lower alkyl; R₅ is hydrogen, lower alkyl or trifluoromethyl; R₆ is hydrogen; R₇ is nitro, fluoro-substituted lower alkoxy or a radical of formula:
   —N(R₉)—C(═X)—(Y)n—R₁₀
wherein R₉ is hydrogen, X is oxo, Y is oxygen or the group NH, n is the number 0 and R₁₀ is pyridyl bonded at a carbon atom, phenyl that is unsubstituted or substituted by halogen, cyano, lower alkoxy, carboxy, lower alkyl or by 4-methyl-piperazinyl-methyl, or C5-C7 alkyl, thienyl, 2-naphthyl or cyclohexyl, and R₈ is hydrogen, or a pharmaceutically acceptable salt of such a compound having at least one salt-forming group. R₄ and R₈ can be hydrogen and the remaining substituents re as defined above, or a pharmaceutically acceptable salt of such a compound having at least one salt-forming group. 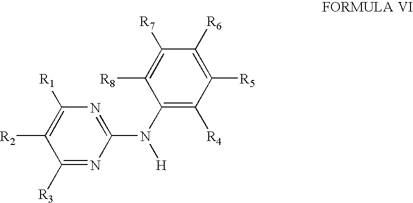
wherein R₁ is pyridyl bonded at a carbon atom; R₂, R₃, R₄, R₅, R₆ and R₈ are each hydrogen and R₇ is nitro or a radical of formula:
   —N(R₉)—C(═X)—(Y)n—R₁₀
wherein R₉ is hydrogen, X is oxo, Y is oxygen or the group NH, n is the number 0; and R₁₀ is pyridyl bonded at a carbon atom, phenyl that is unsubstituted or substituted by fluorine, chlorine, cyano, lower alkoxy, carboxy, lower alkyl or by 4-methyl-piperazinyl-methyl, or C5-C7 alkyl, thienyl or cyclohexyl, or a pharmaceutically acceptable salt thereof. 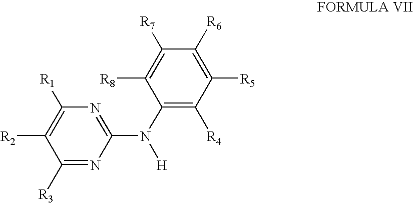
wherein R₁ is 4-pyridyl, N-oxido-4-pyridyl, or 3-indolyl, and R₇ is fluoro-substituted alkoxy containing up to 2 carbon atoms, or a salt of such a compound containing at least one salt-forming group. 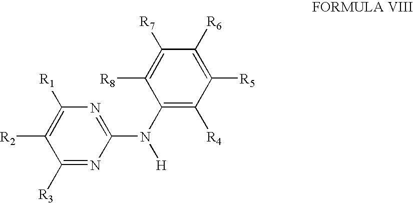
wherein R₁ is 4-pyridyl, N-oxido-4-pyridyl, or 3-indolyl; and R₇ is trifluoromethoxy or 1,1,2,2-tetrafluoroethoxy, or a salt of such a compound containing at least one salt-forming group. Acceptable salt forming groups include but not limited to N-(5-Benzoylamido-2-methyl-phenyl)-4-(3-pyridyl)-2-pyrimidine-amine; and N-[3-(1,1,2,2-Tetrafluoroethoxy)phenyl]-4-(4-pyridyl)-2-pyrimidine-amine. 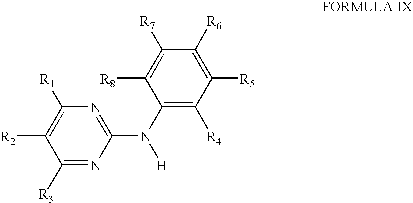
or a pharmaceutically acceptable salt of such a compound having at least one salt-forming group selected from the group consisting of:
1. N-(3-Nitro-phenyl)-4-(3-pyridyl)-2-pyrimidine-amine,
2. N-[3-(4-Chlorobenzoylamido)-phenyl]-4-(3-pyridyl)-2-pyrimidine-amine,
3. N-(3-Benzoylamido-phenyl)-4-(3-pyridyl)-2-pyrimidine-amine,
4. N-[3-(2-Pyridyl)carboxamido-phenyl]-4-(3-pyridyl)-2-pyrimidine-amine,
5. N-[3-(3-pyridyl)carboxamido-phenyl]-4-(3-pyridyl)-2-pyrimidine-amine,
6. N-[3-(4-pyridyl)carboxamido-phenyl]-4-(3-pyridyl)-2-pyrimidine-amine,
7. N-(3-Pentafluoro-benzoylamido-phenyl)-4-(3-pyridyl)-2-pyrimidine-amine,
8. N-[3-(2-Carboxy-benzoylamido)-phenyl]-4-(3-pyridyl)-2-pyrimidine-amine,
9. N-(3-n-Hexanoylamido-phenyl)-4-(3-pyridyl)-2-pyrimidine-amine,
10. N-(3-Nitro-phenyl)-4-(2-pyridyl)-2-pyrimidine-amine,
11. N-(3-Nitro-phenyl)-4-(4-pyridyl)-2-pyrimidine-amine,
12. N-[3-(2-Methoxy-benzoylamido)-phenyl]-4-(3-pyridyl)-2-pyrimidine-amine,
13. N-[3-(4-Fluoro-benzoylamido)-phenyl]-4-(3-pyridyl)-2-pyrimidine-amine,
14. N-[3-(4-Cyano-benzoylamido)-phenyl]-4-(3-pyridyl)-2-pyrimidine-amine,
15. N-[3-(2-Thienylcarboxamido)-phenyl]-4-(3-pyridyl)-2-pyrimidine-amine,
16. N-(3-Cyclohexycarboxamido-phenyl)-4-(3-pyridyl)-2-pyrimidine-amine,
17. N-[3-(4-Methyl-benzoylamido)-phenyl]-4-(3-pyridyl)-2-pyrimidine-amine,
18. N-[3-(4-Chloro-benzoylamido)-phenyl]-4-(4-pyridyl)-2-pyrimidine-amine,
19. N-{3-[4-(4-Methyl-piperazinomethyl)-benzoylamido]-phenyl}-4-(3-pyridyl)-2-pyrimidine-amine,
20. N-[5-(4-Methyl-benzoylamido)-2-methyl-phenyl]-4-(3-pyridyl)-2-pyrimidine-amine,
21. N-[5-(2-Naphthoylamido)-2-methyl-phenyl]-4-(3-pyridyl)-2-pyrimidine-amine,
22. N-[5-(4-Chloro-benzoylamido)-2-methyl-phenyl]-4-(3-pyridyl)-2-pyrimidine-amine,
23. N-[5-(2-Methoxy-benzoylamido)-2-methyl-phenyl]-4-(3-pyridyl)-2-pyrimidine-amine,
24. N-(3-Trifluoromethoxy-phenyl)-4-(3-pyridyl)-2-pyrimidine-amine,
25. N-(3-[1,1,2,2-tetrafluoro-ethoxy]-phenyl)-4-(3-pyridyl)-2-pyrimidine-amine,
26. N-(3-Nitro-5-methyl-phenyl)-4-(3-pyridyl)-2-pyrimidine-amine,
27. N-(3-Nitro-5-trifluoromethyl-phenyl)-4-(3-pyridyl)-2-pyrimidine-amine,
28. N-(3-Nitro-phenyl)-4-(N-oxido-3-pyridyl)-2-pyrimidine-amine,
29. N-(3-Benzoylamido-5-methyl-phenyl)-4-(N-oxido-3-pyridyl)-2-pyrimidine-amine. 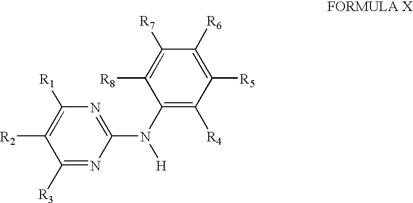
or a pharmaceutically acceptable salt of such a compound having at least one salt-forming group selected from the group consisting of N-[3-(1,1,2,2-tetrafluoroethoxy)phenyl]-4-(N-oxido-4-pyridyl)-2-pyrimidine-amine, and N-[3-(1,1,2,2-tetrafluoroethoxy)phenyl]-4-(3-indolyl)-2-pyrimidine-amine. 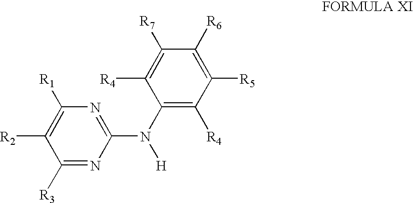
said compound being N-{5-[4-(4-Methyl-piperazino-methyl)-benzoylamido]-2-methyl-phenyl}-4-(3-pyridyl)-2-pyrimidine-amine or a pharmaceutically acceptable salt thereof.

The target of imatinib is preferentially BCR-ABL, an intracellular oncogenic tyrosine kinase that shares several homologies with the class III receptor tyrosine kinase (RTK) family, whose members include the FLT3, KIT, FMS, and PDGF receptors. Most of these RTKs are implicated, either in mutated or wild-type conformations, in the constitutive activation and proliferation of human leukemias, especially acute myeloid leukemia (AML). New Tyrosine Kinase Inhibitors that have been recently identified with structures similar to or different from imatinib are 4-[6-methoxy-7-(3-piperidine-1-yl-propoxy)-quinazolin-4-yl]-piperazine-1-carboxylicacid(4-isopropoxyphenyl)amide (CT53518 or MLN518 from Millennium Pharmaceutical), 5-Chloro-3-[(3,5-dimethylpyrrol-2-yl)methylene]-2-indolinone (SU6656), 5-Chloro-3-[(3,5-dimethylpyrrol-2-yl)methylene]-2-indolinone (SU5614 from Sugen), a water-soluble N,N-dimethylgly-cine ester prodrug CEP7055 that converts to CEP5214 in vivo from Cephalon, West Chester Pa., 4-Amino-5-(4-chlorophenyl)-7-(t-butyl)pyrazolo[3,4-d]pyrimidine (PP2 or AGI 879), 6,7-Dimethyl-2-phenylquinoxaline (AG1295), TAUTOMYCIN™, Radicicol, Damnacanthal, Herbimycin A, 6-(2,6-dichloro-phenyl)-8-methyl-2-(3-methylsulfanyl-phenylamino)-8h-pyrido(2,3-d)pyrimidin-7-one (PD173955 from Parke-Davis), PD166326, PD183805, 4-[(3-Bromophenyl)amino]-6-propionylamidoquinazoline (PD 174265), 5-Chloro-3-[(3,5-dimethylpyrrol-2-yl)methylene]-2-indolinone (PD153035), 4-[(3-Bromophenyl)amino]-6-acrylamidoquinazoline (PD168393), TARCEVA™ (erlotinib HCl), C₁₋₁₀₃₃, AEE788, CP-724,714 (from OSI Pharmaceutical), Geldanamycin, 17-(allylamino)-17-demethoxygeldanamycin (17-AG or 12-AAG), TARCEVA™, IRESSA™, ZD4910.

Different classes of CDK-targeting drugs (cyclin-dependent kinase inhibitor) are cell cycle regulators. They include butyrolactone I substituted purines (e.g., olomoucine, CGP74514, and its derivatives), polyhydroxylated flavones (e.g., flavopyridol), oxindole inhibitors (e.g., GW-8510, GW-2059, GW-5181), and indolinone derivatives (e.g., SU-5416). The phytoestrogenic flavonoid antioxidants (e.g., silibinin, sylymarin and baicalein) potently inhibit cell-cycle progression in G₁ phase by decreasing the levels of cyclin D1, cyclin E, CDK4, CDK6 and CDK2, coupled with increases in p21^{CiP1} and p27^{Kip1}. Alternatively, selective induction of CDKIs can be achieved by inhibitors of histone deacetylase such as trichostatin A, which increases p21^{Cip1} and p16^{INK4A}, while reducing CDK2 activity. Oxindole inhibitors of the cyclin dependent kinases (CDKs) target the CDK2-ATP binding site. They show great selectivity for the CDK2/cyclin A complex. Other CDK inhibitors include PD-0183812 which is a potent and selective CDK4/cyclinD1 inhibitor.

Stenotic processes exert their greatest effect by targeting particular regulators of the G₁ phase, during which cells respond to extracellular signals by either advancing toward another division or withdrawing into a resting state (G₀). Passage through the critical restriction point (R), which is situated late in G₁, and entry into S phase is controlled by cyclin-dependent (serine/threonine) protein kinases (CDKs), that are sequentially regulated by cyclins (D, E and A) with whom they form active complexes, which phosphorylate important proteins of the cell-cycle control. The CDK activity is constrained by at least two families of CDK inhibitory proteins (CDKIs): the universal Cip/Kip (p21^{CiP1}, p27^{Kip1} and p57^{Kip2}) and the INK4 (p15^{INK4B}, p16^{INK4A}, p18 and p19) families. The cyclin-dependent kinases (CDKs) are important targets for therapeutic intervention in various proliferative disease states including stenosis.

Examples of suitable substituted purine derivatives type CDK inhibitors include compounds of Formulas XII: 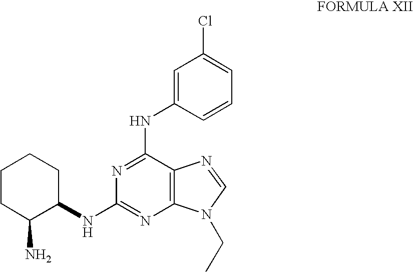
with a chemical name of N²-(cis-2-Aminocyclohexyl)-N⁶-(3-chlorophenyl)-9-ethyl-9H-purine-2,6-diamine hydrochloride or pharmaceutically acceptable salts thereof.

Examples of suitable oxindole derivative type CDK inhibitors include compounds of Formulas XIII: 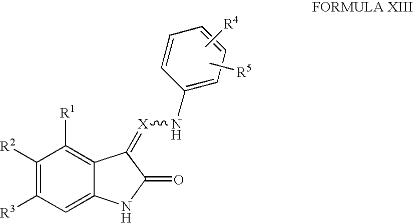
wherein X is N, CH or CCH3; R₁ and R₂ are joined to form a thiazole, pyrazole, triazole, pyridyl, 3-chloro pyrazole or dihydropyrrolone ring; R₃ is hydrogen, bromo, chloro, methyl, ethyl, isopropyl, hydroxy, hydroxymethyl, phenoxy, or ethoxy; R₄ is in the para-position of the phenyl ring relative to the NH group, and is selected from aminosulfonyl, N-methylaminosulfonyl, N,N-dimethylaminosulfonyl, aminosulfonylamino, N-hydroxyethoxyethylaminosulfonyl, N-hydroxyethylaminosulfonyl, N-(3-hydroxy-2,2-dimethyl-propyl)aminosulfonyl-methyl, N-methylaminosulfonyl-methyl, N-amino-imino methyl-aminosulfonyl, aminosulfonyl-methyl, N-allylaminosulfonyl-methyl, methylsulfonylmethyl, N-(3-hydroxy-2,2-dimethyl-propyl)aminosulfonyl, N-methylcarbonylaminosulfonyl, N-hydroxyethoxyethyl-N-methylaminosulfonyl, and N-methoxyethoxyethoxyethoxyethyl-aminosulfonyl, R₅ is hydrogen, and the pharmaceutically acceptable salts, biohydrolyzable esters, biohydrolyzable amides, biohydrolyzable carbamates, solvates, or hydrates, thereof in either crystalline or amorphous form.

Examples of suitable indolinone derivative type CDK inhibitors include compounds of Formulas XIV: 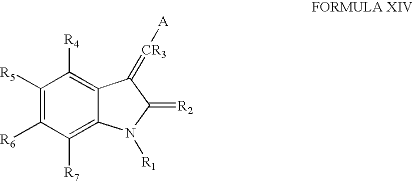
and pharmaceutically acceptable salts thereof, wherein R₁ is H or alkyl; R₂ is O or S; R₃ is hydrogen; R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO2NRR′, SO3R, SR, NO2, NRR′, OH, CN, C(O)R, OC(O)R, NHC(O)R, (CH2)nCO2R, and CONRR′, A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3-thiatriazole, and tetrazole, optionally substituted at one or more positions with alkyl, alkoxy, aryl, aryloxy, alkaryl, alkaryloxy, halogen, trihalomethyl, S(O)R, SO₂ NRR′, SO₃R, SR, NO₂, NRR′, OH, CN, C(O)R, OC(O)R, NHC(O)R, (CH2)nCO2R or CONRR′, n is 0-3, X is Br, Cl, F or I, R is H, alkyl or aryl, and R′ is H, alkyl or aryl.

NF-κB Decoy Oligo has a role in excessive expression of adhesion molecules and cytokines related to immunity and inflammation. It is typically attached to inhibitor-kappaB (I κB) in the cytoplasm; IκB stops NF-κB from moving into the nucleus. Once IκB is phosphorylated and degraded, NF-κB is activated to enter into the nucleus. NF-κB is bound to the NF-κB binding site on the chromosome to promote transcription of the downstream gene. The genes regulated by NF-κB include cytokines and adhesion molecules involved ion immunity and inflammation. NF-κB decoy oligo is a 20-base DNA consisting of the sequence in the region of the NF-κB binding site on the chromosome. It blocks binding of NF-κB with the chromosome, resulting in inhibition of excessive expression of cytokines or adhesion molecules. NF-κB decoy oligo was named after its decoy-like action.

IGF-1 is an insulin like growth factor that regulates essential metabolic and anabolic growth promoting processes, such as glucose uptake and tissue regeneration. There are at least six different IGF binding proteins (“IGFBP”), with IGFBP-3 binding to over 90% of IGF in the body. Disregulation of IGF/IGFBP ratios can have a major role to play in diseases such as cancer and other diseases such as atherosclerosis and hyperproliferative diseases like restenosis. Recombinant protein, rhlGFBP-3 (Manufactured by Insmed Inc.) has been shown to induce cancel cell death in a variety of experimental systems. Several studies have demonstrated that cancer risk increases with decreasing levels of circulating IGFBP-3. In addition, recent independent studies have demonstrated that IGFBP-3 can induce cell cycle arrest and enhance the efficacy of chemotherapeutic agents. It is proposed that IGF, IGF-1, IGFBP, IGFBP-3, rhlGFBP-3, alone or in combination with each other or additional drugs described herein may used on a stent to treat hyperproliferative disease.

In an embodiment, the source of the therapeutic capable agent is a polymeric material including therapeutic capable agent moieties as a structural subunit of the polymer. The therapeutic capable agent moieties are polymerized and associated to one another through suitable linkages (e.g., ethylenic) forming polymeric therapeutic capable agent. Once the polymeric therapeutic capable agent is brought into contact with tissue or fluid such as blood, the polymeric therapeutic capable agent subunits disassociate. Alternatively, the therapeutic capable agent may be released as the polymeric therapeutic capable agent degrades or hydrolyzes, preferably, through surface degradation or hydrolysis, making the therapeutic capable agent available to the susceptible tissue site, preferably over a period of time. Examples of methods and compounds for polymerizing therapeutic capable agents are described in WO 99/12990 Patent Application by Kathryn Uhrich, entitled “Polyanhydrides With Therapeutically Useful Degradation Products,” and assigned to Rutgers University, the full disclosure of which is incorporated herein by reference. Examples of a therapeutic capable agent and a suitable reaction ingredient unit include mycophenolic acid with adipic acid and/or salicylic acid in acid catalyzed esterification reaction, mycophenolic acid with aspirin and/or adipic acid in acid catalyzed esterification reaction, mycophenolic acid with other NSAIDS, and/or adipic acid in acid catalyzed esterification reaction. In an embodiment, the polymeric therapeutic capable agent may be associated with a polymeric and/or metallic backbone.

The expandable structure 16, as shown without intending any limitation, has a tissue facing surface 31 and a luminal facing surface 34, and optionally an interior 37 which may include a lumen as shown in FIG. 2B. It will be appreciated that the following depictions are for illustration purposes only and do not necessarily reflect the actual shape, size, configuration, or distribution of the prosthesis 13. The prosthesis may have a continuous structure or an intermittent structure as the case may be with many stents (e.g., a cross section of a stent does not entirely include a substrate forming the expandable structure, for example, some stents have a screen or mesh like cross section). The source may be disposed or formed adjacent at least a portion of either or both the luminal facing surface, as shown in FIG. 1B, the tissue facing surface, as shown in FIG. 1C, within the interior of the expandable structure, and/or any combination thereof. In an embodiment, devices may be configured to make available to the tissue the most suitable therapeutic amount of the therapeutic capable agent while minimizing the presence of unwanted metabolites and by-products of the therapeutic capable agent at the tissue site.

The source 25, for making the therapeutic capable agent available, is associated with the expandable structure in one or more configurations. The source as shown in FIGS. 2A and 2B, is within the expandable structure 16, as for example, when a matrix 40 is formed by the expandable structure 16 and the therapeutic capable agent 28, or when the therapeutic capable agent 28 is disposed within the interior 37 (or the exterior of the expandable structure 16 as the case may be) of the expandable structure 16. Now referring to FIG. 2C, the source may further comprises a rate-sustaining or rate-controlling element 43 formed over at least a portion of the expandable structure 16 for sustaining or controlling the release of the therapeutic capable agent 28 from the matrix 40 or the interior 37 of the expandable structure. By way of example, the source may be the rate-sustaining or rate-controlling element itself when the therapeutic capable agent is a polymeric therapeutic capable agent.

The rate-sustaining or rate-controlling element may be formed of a non-degradable, partially degradable, substantially degradable material, or a combination thereof. The material may be synthetic or natural; non-polymeric, polymeric or metallic; bio-active or non bio-active compounds; or a combination thereof. By way of examples, a metallic material that at least partially degrades with time may be used as the rate-sustaining or rate-controlling element; as well as non-polymers having large molecular weight, polar or non-polar functional groups, electrical charge, steric hindrance groups, hydrophobic, hydrophilic, or amphiphilic moieties.

Suitable biodegradable rate-sustaining or rate-controlling element materials include, but are not limited to, poly(lactic acid), poly(glycolic acid) and copolymers, poly dioxanone, poly(ethyl glutamate), poly(hydroxybutyrate), polyhydroxyvalerate and copolymers, polycaprolactone, polyanhydride, poly(ortho esters), poly(iminocarbonates), polyester-amids, polycyanoacrylates, polyphosphazenes, copolymers and other aliphatic polyesters, or suitable copolymers thereof including copolymers of poly-L-lactic acid and poly-e-caprolactone, and mixtures, copolymers, and combinations thereof. Other suitable examples of biodegradable rate-sustaining or rate-controlling element include polyamide esters made from amino acids (such as L-lysine and l-leucine) along with other building blocks such as diols (hexanediol) and diacids (such as sebacic acid, as described in another embodiment). The therapeutic capable agent may be released either from a reservoir or a matrix comprising the above polymer. The therapeutic capable agent may be also covalently attached to the amino acids and released as the polymer biodegrades. Other biodegradable poly ester urethanes made from copolymers of poly lactide, poly caprolactone, poly ethylene glycol, polyester-amid, and poly acrylic acid can also be used to release the therapeutic capable agent as described above.

An example of a biodegradable material of the present invention is a copolymer of poly-L-lactic acid (having an average molecular weight of about 200,000 daltons) and poly-e-caprolactone (having an average molecular weight of about 30,000 daltons). Poly-e-caprolactone (PCL) is a semi crystalline polymer with a melting point in a range from 59° C. to 64° C. and a degradation time of about 2 years. Thus, poly-l-lactic acid (PLLA) can be combined with PCL to form a matrix that generates the desired release rates. A preferred ratio of PLLA to PCL is 75:25 (PLLA/PCL). As generally described by Rajasubramanian et al. in ASAIO Journal, 40, pp. M584-589 (1994), the full disclosure of which is incorporated herein by reference, a 75:25 PLLA/PCL copolymer blend exhibits sufficient strength and tensile properties to allow for easier coating of the PLLA/PLA matrix on the expandable structure. Additionally, a 75:25 PLLA/PCL copolymer matrix allows for sustained or controlled drug delivery over a predetermined time period as a lower PCL content makes the copolymer blend less hydrophobic while a higher PLLA content leads to reduced bulk porosity.

Suitable nondegradable or slow degrading rate-sustaining or rate-controlling element materials include, but are not limited to, polyurethane, polyethylene, polyethylenes imine, cellulose acetate butyrate, ethylene vinyl alcohol copolymer, silicone, polytetrafluorethylene (PTFE), parylene, parylene C, N, D, or F, parylene C, PARYLAST™, PARYLAST™ C, poly(methyl methacrylate butyrate), poly-N-butyl methacrylate, poly (methyl methacrylate), poly 2-hydroxy ethyl methacrylate, poly ethylene glycol methacrylates, poly vinyl chloride, poly(dimethyl siloxane), poly(tetrafluoroethylene), poly (ethylene oxide), poly ethylene vinyl acetate, poly carbonate, poly acrylamide gels, N-vinyl-2-pyrrolidone, maleic anhydride, Nylon, cellulose acetate butyrate (CAB) and the like, including other synthetic or natural polymeric substances, and mixtures, copolymers, and combinations thereof. In an embodiment the rate-sustaining or rate-controlling element is formed from a material selected from the group consisting of silicone, polytetrafluoroethylene, parylene, parylene C, non-porous parylene C, PARYLAST™, PARYLAST™C, polyurethane, cellulose acetate butyrate, and mixtures, copolymers and combinations thereof. These polymers can have a foam structure, porous structure, nano-porous structure, non-porous structure, structure with cracks, openings, fissures, perforations or combinations thereof.

Suitable natural material include, but are not limited to, fibrin, albumin, collagen, gelatin, glycosoaminoglycans, oligosaccharides & poly saccharides, chondroitin, phosholipids, phosphorylcholine, glycolipids, proteins, oligomers, amino acids, peptides, cellulose, and mixtures, copolymers, or combinations thereof. Other suitable materials include titanium, chromium, Nitinol, gold, stainless steel, metal alloys, or a combination thereof as well as other compounds that may release the therapeutic capable agent as a result of interaction (e.g., chemical reaction, high molecular weight, steric hindrance, hyrophobicity, hydrophilicity, amphilicity, heat) of the therapeutic capable agent with the rate-sustaining or rate-controlling element material (e.g, a non-polymer compound). By way of example, a combination of two or more metals or metal alloys with different galvanic potentials to accelerate corrosion by galvanic corrosion pathways may also be used.

The degradable material may degrade by bulk degradation or hydrolysis. In an embodiment, the rate-sustaining or rate-controlling element degrades or hydrolyzes throughout, or preferably, by surface degradation or hydrolysis, in which a surface of the rate-sustaining or rate-controlling element degrades or hydrolyzes over time while maintaining bulk integrity. In another embodiment, hydrophobic rate-sustaining or rate-controlling elements are preferred as they tend to release therapeutic capable agent at desired release rate. A non-degradable rate-sustaining or rate-controlling element may release therapeutic capable agent by diffusion. By way of example, if the rate-sustaining or rate-controlling element is formed of non-polymeric material, the therapeutic capable agent may be released as a result of the interaction (e.g., chemical reaction, high molecular weight, steric hindrance, hyrophobicity, hydrophilicity, amphilicity, heat) of the therapeutic capable agent with the rate-sustaining or rate-controlling element material (e.g, a non-polymer compound). In an embodiment, when the rate-sustaining or rate-controlling element does not form, at least a sufficient matrix with the therapeutic capable agent, the therapeutic capable agent may be released by diffusion through the rate-sustaining or rate-controlling element. By way of example, a rate-sustaining or rate-controlling element having low molecular weight and/or relatively high hydrophilicity in the tissue or blood, may diffuse through the source (e.g., a matrix). This increases the surface area or volume for the therapeutic capable agent to be released from, thus, affecting the release rate of the therapeutic capable agent.

FIG. 2D illustrates features of an embodiment having the therapeutic capable agent 28 disposed between one of the tissue or luminal facing surfaces of the expandable structure 16 and the rate-sustaining or rate-controlling element 43. As shown in FIG. 2E, the source 25 includes the rate-sustaining or rate-controlling element 43 formed adjacent at least a portion of one of the tissue or luminal facing surfaces of the expandable structure 16 and forming the matrix 40 with the therapeutic capable agent 28. As noted earlier, the therapeutic capable agent 28 may itself act as a rate-sustaining or rate-controlling element, as for example, when the polymeric therapeutic capable agent forms a matrix. The matrix may be formed between the rate-sustaining or rate-controlling element 43 and the expandable structure 16 and forming a matrix interface 46 therebetween and/or between the therapeutic capable agent 28 and the rate-sustaining or rate-controlling element 43, as shown in FIGS. 2F and 2G respectively.

In an embodiment, features of which are shown in FIG. 2H, the outer most layer of the prosthesis 13 may be formed of the therapeutic capable agent with or without a matrix interface 46 formed between the outer most layer and the other layers. It should be noted that the therapeutic capable agent 28, although as shown in most figures as discrete particles, may form a smooth layer or a layer of particles, as for example as part of matrix interface 46 as shown in FIG. 2H.

In an alternate embodiment, features of which are shown in FIG. 21, at least one layer of a second rate-sustaining or rate-controlling element 49 is formed over the matrix 40, further affecting the release rate of the therapeutic capable agent 28 to the susceptible tissue site. The second rate-sustaining or rate-controlling element 49 may be of the same or different material than that forming the first rate-sustaining or rate-controlling element 43.

Now referring to FIGS. 2J and 2K, the source may comprise a plurality of compounds, as for example the first therapeutic capable agent 28 and an optional another compound 50, such as another or second therapeutic capable agent 50 or an enabling compound 61 (FIG. 2N). Each of the plurality of compounds may be in the same or different area of the source. For example, as shown in FIG. 2K, the first therapeutic capable agent 28 may be present in matrix 40 while the second therapeutic capable agent 50 is in a second matrix 52 formed by the second therapeutic capable agent 50 and a second rate-sustaining or rate-controlling element 55. The rate-sustaining or rate-controlling elements 43 and 55 may be formed from the same or different material. The another or second therapeutic capable agent may act in synergy with the first therapeutic capable agent. For example, the second therapeutic capable agent may compensate for the possible reactions and by-products that can be generated by the first therapeutic capable agent. By way of example, the therapeutic capable agent may reduce generation of desired endothelial cells while a suitable optional another therapeutic capable agent may allow for more endothelialization to be achieved. The another therapeutic agent may be released prior to, concurrent with, or subsequent to, the therapeutic capable agent, at similar or different rates and phases.

The another therapeutic capable agent may comprise at least one compound selected from the group consisting of anti-cancer agents; chemotherapeutic agents; thrombolytics; vasodilators; antimicrobials or antibiotics antimitotics; growth factor antagonists; free radical scavengers; biologic agents; radiotherapeutic agents; radiopaque agents; radiolabelled agents; anti-coagulants such as heparin and its derivatives; anti-angiogenesis drugs such as THALIDOMIDE™; angiogenesis drugs; PDGF-B and/or EGF inhibitors; anti-inflamatories including psoriasis drugs; riboflavin; tiazofurin; zafurin; anti-platelet agents including cyclooxygenase inhibitors such as acetylsalicylic acid; ADP inhibitors such as clopidogrel (e.g., PLAVIX™) and ticlopdipine (e.g., TICLID™); phosphodiesterase III inhibitors such as cilostazol (e.g., PLETAL™); glycoprotein IIb/IIIa agents such as abciximab (e.g., RHEOPRO™); eptifibatide (e.g., INTEGRILIN™); adenosine reuptake inhibitors such as dipyridmoles; healing and/or promoting agents including anti-oxidants; nitrogen oxide donors; antiemetics; antiauseants; phenylaminopyrimidine (or phenylpyrimidine-amine) derivatives (e.g., Imatinib (GLIVEC™)), silibinin, sylymarin, baicalein, histone deacetylase such as trichostatin A, PD-0183812, butyrolactone I substituted purines (e.g., olomoucine, CGP74514, and its derivatives), polyhydroxylated flavones (e.g., flavopyridol), oxindole inhibitors (e.g., GW-8510, GW-2059, GW-5181), and indolinone derivatives (e.g., SU-5416),), Zoledronic acid (i.e., ZOMETA™, Zoledronic acid, and (1-Hydroxy-2-imidazol-1-yl-phosphonoethyl)phosphonic acid monohydrate), other tyrosine inhibitors such as 4-[6-methoxy-7-(3-piperidine-1-yl-propoxy)-quinazolin-4-yl]-piperazine-1-carboxylicacid(4-isopropoxyphenyl)amide (CT53518 or MLN518 from Millennium Pharmaceutical), 5-Chloro-3-[(3,5-dimethylpyrrol-2-yl)methylene]-2-indolinone (SU6656), 5-Chloro-3-[(3,5-dimethylpyrrol-2-yl)methylene]-2-indolinone (SU5614 from Sugen), a water-soluble N,N-dimethylgly-cine ester prodrug CEP7055 that converts to CEP5214 in vivo from Cephalon, West Chester Pa., 4-Amino-5-(4-chlorophenyl)-7-(t-butyl)pyrazolo[3,4-d]pyrimidine (PP2 or AG1879), 6,7-Dimethyl-2-phenylquinoxaline (AG1295), TAUTOMYCIN™, Radicicol, Damnacanthal, Herbimycin A, 6-(2,6-dichloro-phenyl)-8-methyl-2-(3-methylsulfanyl-phenylamino)-8h-pyrido(2,3-d)pyrimidin-7-one (PD173955 from Parke-Davis), PD166326, PD183805, 4-[(3-Bromophenyl)amino]-6-propionylamidoquinazoline (PD 174265), 5-Chloro-3-[(3,5-dimethylpyrrol-2-yl)methylene]-2-indolinone (PD153035), 4-[(3-Bromophenyl)amino]-6-acrylamidoquinazoline (PD168393), TARCEVA™ (erlotinib HCl), CI-1033, AEE788, CP-724,714 (from OSI Pharmaceutical), Geldanamycin, 17-(allylamino)-17-demethoxygeldanamycin (17-AG or 12-AAG), TARCEVA™, IRESSA™, and ZD4910, EGFR/ErbB2 inhibitor (CI1033; EKB569; GW2016; PKI166), VEGF receptor inhibitors (ZK222584;ZD6474), VEGFR/FGFR/PDGFR inhibitors (SU6668; SU11248; PTK787), NGF receptor (CEP2583), anti-EGF receptor MAbs (MAb225/ERBITUX™), anti-ErbB2 MAbs (MAb4D5/HERCEPTIN™), AVASTIN™, an anti-VEGF MAb, NF-κB Decoy Oligo; proteins such as albumin; genes such as TSC1, TSC2, hamartin, or KIAA0243; growth factors such as VEGF, EGF, PDGF, or FGF; anti-sense such as antisense phosphorothioate oligodeoxynucleotide (ODN); anti-bodies such as Anti-MTOR, Anti-p27 Anti-p53, or Anti-Cdk; derivatives, agent incorporated in a vector such as a HVJ Envelop vector; derivatives and combinations thereof.

In another embodiment, features of which are shown in FIGS. 2L and 2M, the therapeutic capable agent 28 is disposed within or on the expandable structure 16 within a reservoir 58. The rate-sustaining or rate-controlling element 43 may be disposed adjacent the reservoir 58 and/or the therapeutic capable agent 28 for affecting the release of the therapeutic capable agent. As stated earlier, the exemplary figures and descriptions are not meant to limit the term “adjacent.”

In a further embodiment, features of which are shown in FIG. 2N, the another optional compound comprises an enabling compound 61 responsive to an external form of energy, or native condition, to affect the release of the therapeutic capable agent. The responsive compound may be associated with the therapeutic capable agent, the rate-sustaining or controlling element, the expandable structure, or a combination thereof. As shown in FIG. 2N, the responsive compound is associated with the therapeutic capable agent. The enabling compound 61 may be formed from magnetic particles coupled to the therapeutic capable agent 28. The energy source may be a magnetic source for directing a magnetic field at the prosthesis 13 after implantation to effect release of the therapeutic capable agent 28. The magnetic particles 61 may be formed from magnetic beads and will typically have a size in a range from about 1 nm to about 100 nm. The magnetic source exposes the prosthesis 13 to its magnetic field at an intensity typically in the range from about 0.01T to about 2T, which will activate the magnetic particles 61 and thereby effect release of the therapeutic capable from the prosthesis. The another enabling compound may be present in other configurations of prosthesis 13 as described above. Other suitable external energy sources, which may or may not require an enabling compound or their performance may not be affected by the presence or absence of an enabling compound, include ultrasound, magnetic resonance imaging, magnetic field, radio frequency, temperature change, electromagnetic, x-ray, radiation, heat, gamma, vibration, microwave, or a combination thereof.

By way of example, an ultrasound external energy source may be used having a frequency in a range from 20 kHz to 100 MHz, preferably in a range from 0.1 MHz to 20 MHz, and an intensity level in a range from 0.05 W/cm² to 10 W/cm², preferably in a range from 0.5 W/cm² to 5 W/cm². The ultrasound energy may be directed at the prosthesis 13 from a distance in a range from 1 mm to 30 cm, preferably in a range from 1 cm to 20 cm. The ultrasound may be continuously applied or pulsed, for a time period in a range from 5 sec to 30 minutes, preferably in a range from 1 minute to 15 minutes. The temperature of the prosthesis 13 during this period will be in a range from 36° C. to 48° C. The ultrasound may be used to increase a porosity of the prosthesis 13, thereby allowing release of the therapeutic capable agent 28 from the prosthesis 13. Other sources of energy, for example, heat or vibrational energy, may also be used to increase the porosity of the prosthesis or a portion thereof, or alter the configuration of the same.

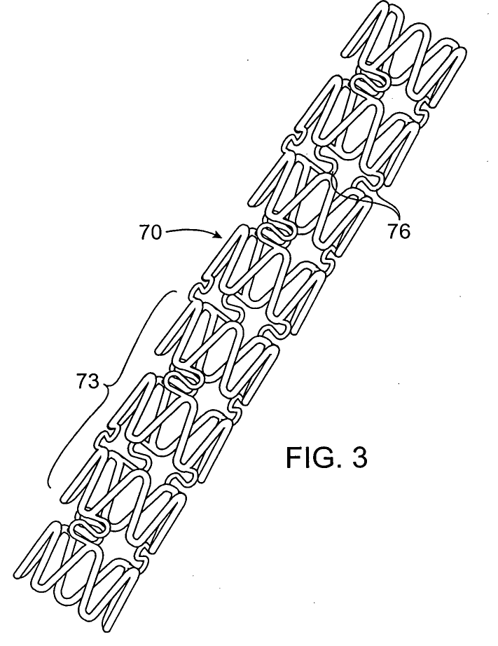

Now referring to FIG. 3, the expandable structure 16 may be a stent 70 or a graft (not shown). When the expandable structure is a stent, the expandable structure 16 will usually comprise at least two radially expandable, usually cylindrical, ring segments 73 as shown in FIG. 3. Typically, the expandable structure 16 will have at least four, and often five, six, seven, eight, ten, or more ring segments. At least some of the ring segments will be adjacent to each other but others may be separated by other non-ring structures. The description of exemplary stent structures is not intended to be exhaustive and it should be appreciated that other variations of stent designs may be used in the present invention.

Referring back to FIG. 3, an exemplary stent 70 (embodying features of a stent described in more detail in co-pending U.S. patent application Ser. No. 08/968,319) for use in the present invention comprises from 4 to 50 ring segments 73 (with eight being illustrated). Each ring segment 73 is joined to the adjacent ring segment by at least one of sigmoidal links 76 (with three being illustrated). Each ring segment 73 includes a plurality of strut/hinge units, e.g., six strut/hinge units, and three out of each six hinge/strut structures on each ring segment 73 will be joined by the sigmoidal links 76 to the adjacent ring segment. As shown in FIG. 3, stent 70 is in a collapsed or non-expanded configuration.

As used herein, the term “radially expandable” includes segments that can be converted from a small diameter configuration to a radially expanded, usually cylindrical, configuration which is achieved when the expandable structure 16 is implanted at a desired target site. The expandable structure 16 may be minimally resilient, e.g., malleable, thus requiring the application of an internal force to expand and set it at the target site. Typically, the expansive force can be provided by a balloon, such as the balloon of an angioplasty catheter for vascular procedures. The expandable structure 16 preferably provides sigmoidal links between successive unit segments to enhance flexibility and crimpability of the stent.

Alternatively, the expandable structure 16 can be self-expanding. Self-expanding structures are provided by utilizing a resilient material, such as a tempered stainless steel, or a superelastic alloy such as a nitinol alloy, and forming the body segment so that it possesses a desired radially-expanded diameter when it is unconstrained, i.e. released from the radially constraining forces of a sheath. In order to remain anchored in the body lumen, the expandable structure 16 will remain partially constrained by the lumen. The self-expanding expandable structure 16 can be tracked and delivered in its radially constrained configuration, e.g., by placing the expandable structure 16 within a delivery sheath or tube and removing the sheath at the target site.

The dimensions of the expandable structure will depend on its intended use. Typically, the expandable structure will have a length in a range from about 5 mm to about 100 mm, usually being from about 8 mm to about 50 mm, for vascular applications. The diameter of a cylindrically shaped expandable structure for vascular applications, in a non-expanded configuration, usually ranges from about 0.5 mm to about 10 mm, more usually from about 0.8 mm to about 8 mm; with the diameter in an expanded configuration ranging from about 1.0 mm to about 100 mm, preferably from about 2.0 mm to about 30 mm. The expandable structure usually will have a thickness in a range from about 0.025 mm to 2.0 mm, preferably from about 0.05 mm to about 0.5 mm.

The ring segments, and other components of the expandable structure 16, may be formed from conventional materials used for body lumen stents and grafts, typically being formed from malleable metals or alloys, such as 300 series stainless steel, from resilient metals, such as superelastic and shape memory alloys (e.g., Nitinol™ alloys, spring stainless steels, and the like), non-metallic materials, such as polymeric materials, or a combination thereof. The polymeric materials may include those polymeric materials that are substantially non-degradable, biodegradable, or substantially biodegradable, such as those described in relation to the materials of choice for the rate-sustaining or rate-controlling element. When the expandable structure material is formed of the rate-sustaining or rate-controlling element material, the expandable structure may function both as the prosthesis and the direct source of the therapeutic capable agent. Additional structures that may be incorporated into the expandable structure of the present invention are illustrated in U.S. Pat. Nos. 5,195,417; 5,102,417; and 4,776,337, the full disclosures of which are incorporated herein by reference. Other suitable material for use as the structure include carbon or carbon fiber, cellulose acetate, cellulose nitrate, silicone, polyethylene terphthalate, polyurethane, polyamide, polyester, polyorthoester, polyanhydride, polyether sulfone, polycarbonate, polytetrafluoroethylene, another biocompatible polymeric material, polyanhydride, polycaprolactone, polyhydroxybutyrate valerate, another biodegradable polymer, protein, an extracellular matrix component, collagen, fibrin, another biologic agent, or a suitable mixture or copolymer of any of the materials listed above, degradable, non-degradable, metallic, or otherwise. In an embodiment, the device may comprise a biodegradable structure with a polymeric source, such as a polymeric therapeutic capable agent.

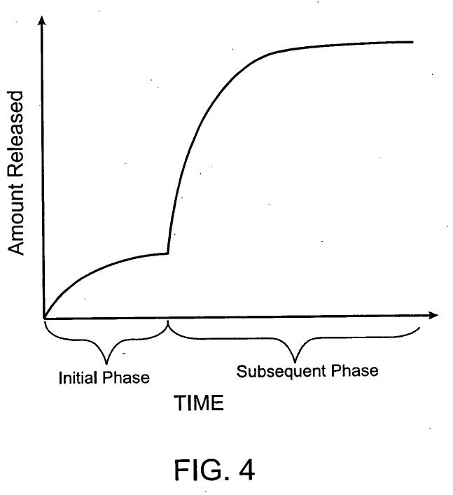

Referring now to FIG. 4, a graphical representation of an exemplary embodiment of therapeutic capable agent release over a predetermined time period is shown. The predetermined rate pattern shown in FIG. 4 of the present invention improves the efficacy of the delivery of the therapeutic capable agent to the susceptible tissue site by making the therapeutic capable agent available at none to some lower delivery rate during an initial phase. Once a subsequent phase is reached, the delivery rate of the therapeutic capable agent may be substantially higher. Thus, time delayed therapeutic capable agent release can be programmed to impact restenosis (or other targeted conditions as the case may be) when there is at least a partial formation of the initial cellular deposition or proliferation (hyperplasia). The present invention can further reduce the washout of the therapeutic capable agent by timing the release of the therapeutic capable agent to occur after at least initial cellularization. Moreover, the predetermined rate pattern may reduce the loading and/or concentration of the therapeutic capable agent. The predetermined rate pattern may further provide limited or reduced to no hindrance to endothelialization of the vessel wall due to the minimization of washout of the therapeutic capable agent and the increased efficiency of its release.

The devices of the present invention may be configured to release or make available the therapeutic capable agent at one or more phases, the one or more phases having similar or different performance (e.g., release) profiles. The therapeutic capable agent may be made available to the tissue at amounts which may be sustainable, intermittent, or continuous; in one or more phases; and/or rates of delivery; effective to reduce any one or more of smooth muscle cell proliferation, inflammation, immune response, hypertension, or those complementing the activation of the same. Any one of the at least one therapeutic capable agents may perform one or more functions, including preventing or reducing proliferative/restenotic activity, reducing or inhibiting thrombus formation, reducing or inhibiting platelet activation, reducing or preventing vasospasm, or the like.

The total amount of therapeutic capable agent made available to the tissue depends in part on the level and amount of desired therapeutic result. The therapeutic capable agent may be made available at one or more phases, each phase having a similar or different release rate and duration as the other phases. The release rate may be pre-defined. In an embodiment, the rate of release may provide a sustainable level of therapeutic capable agent to the susceptible tissue site. In another embodiment, the rate of release is substantially constant. The rate may decrease and/or increase over time, and it may optionally include a substantially non-release period. The release rate may comprise a plurality of rates. In an embodiment the plurality of release rates include at least two rates selected from the group consisting of substantially constant, decreasing, increasing, substantially non-releasing.

The total amount of therapeutic capable agent made available or released may be in an amount ranging from about 0.1 μg to about 10 g, generally from about 0.1 μg to about 10 mg, usually from about 1 μg to about 10 mg, from about 1 μg to about 5 mg, typically from about 1 μg to about 2 mg, from about 10 μg to about 2 mg, from about 10 μg to about 1 mg, from about 50 μg to about 1 mg, or from about 50 μg to about 500 μg. In an embodiment, the therapeutic capable agent may be released in a time period, as measured from the time of implantig of the device, ranging from about 1 day to about 200 days; from about 1 day to about 45 days; or from about 7 days to about 21 days. In an embodiment the release rate of the therapeutic capable agent per day may range from about 0.001 μg to about 500 μg, from about 0.001 μg to about 200 μg, from about 0.5 μg to about 200 μg, usually, from about 1.0 μg to about 100 μg, from about 1 μg to about 60 μg, and typically, from about 5 μg to about 50 μg.

The therapeutic capable agent may be made available at an initial phase and one or more subsequent phases. When the therapeutic capable agent is delivered at different 30 phases, the initial delivery rate will typically be from about 0 to about 99% of the subsequent release rates, usually from about 0% to about 90%, preferably from about 0% to 75%, more preferably from about 0% to 50%. The device may be configured to release the therapeutic capable agent at an initial phase having a lower rate of release than a subsequent phase. The rate of delivery during the initial phase will typically range from about 0.001 ng per day to about 500 μg per day, from about 0 to about 50 μg per day, usually from about 0.001 ng per day to about 50 μg per day, more usually from about 0.1 μg per day to about 30 μg per day, more preferably, from about 1 μg per day to about 20 μg per day. The rate of delivery at the subsequent phase may range from about 0.01 ng per day to about 500 μg per day, from about 0.01 μg per day to about 200 μg per day, usually from about 1 μg per day to about 100 μg per day. In one embodiment, the therapeutic capable agent is made available to the susceptible tissue site in a programmed, sustained, and/or controlled manner with increased efficiency and/or efficacy. Moreover, the present invention provides limited or reduced hindrance to endothelialization of the vessel wall.

The device may be configured to release the therapeutic capable agent at an initial phase having a higher rate of release than a subsequent phase. The rate of delivery during the initial phase will typically range from about 10 μg per day to about 300 μg per day, usually from about 40 μg per day to about 300 μg per day, more usually from about 40 μg per day to about 200 μg per day. The rate of delivery at the subsequent phase may range from about 0.1 μg per day to about 100 μg per day, usually from about 0.5 μg per day to about 40 μg per day, more usually from about 10 μg per day to about 40 μg per day. Alternatively, the device may be configured to release the therapeutic capable agent at a constant rate ranging from about 0.01 μg per day to about 200 μg per day.

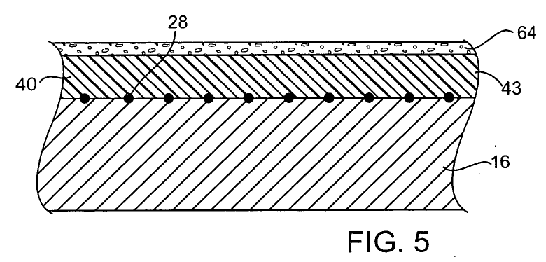

The duration of the initial, subsequent, and any other additional phases may vary. For example, the release of the therapeutic capable agent may be delayed from the initial implantation of the device. Typically, the delay is sufficiently long to allow the generation of sufficient cellularization 64, endothelialization, or fibrin deposition at the treated site and/or device after implantation, as shown in FIG. 5. Typically, the duration of the initial phase will be sufficiently long to allow initial cellularization or endothelialization at, at least part of the device. Typically, the duration of the initial phase, whether being a delayed phase or a release phase, is less than about 24 weeks, from about 1 hour to about 24 weeks, usually less than about 12 weeks, more usually from about 1 hour to about 8 weeks, from about 1 day to about 30 days, more preferably from about 12 hours to about 4 weeks, from about 12 hours to about 2 weeks, from about 1 day to about 2 weeks, or from about 1 day to about 1 week.

The durations of the one or more subsequent phases may also vary, typically being from about 4 hours to about 24 weeks, from about 1 hour to about 12 weeks, from about 1 day to about 12 weeks, from about 1 hour to about 8 weeks, from about 4 hours to about 8 weeks, from about 2 days to about 8 weeks, from about 2 days to about 45 days, more preferably from about of 3 days to about 50 days, from about 3 days to about 30 days, most preferably from about 1 hour to about 1 day. In an embodiment, the duration specified relates to a vascular environment. The more than one phase may include similar or different durations, amounts, and/or rates of release. For example, in one scenario, there may be an initial phase of delay, followed by a subsequent phase of release at a first subsequent rate, and a second subsequent phase of release at a second subsequent rate, and the like.

In an embodiment a mammalian tissue concentration of the substance at an initial phase will typically be within a range from about 0.001 ng/mg of tissue to about 100 μg/mg of tissue; from about 1 ng/mg of tissue to about 100 μg/mg of tissue; from about 10 ng/mg of tissue to about 100 μg/mg of tissue; from about 0.1 ng/mg of tissue to about 50 μg/mg of tissue; from about 1 ng/mg of tissue to about 10 μg/mg of tissue; from about 1 ng/mg of tissue to about 1 μg/mg of tissue. A mammalian tissue concentration of the substance at a subsequent phase will typically be within a range from about 0.001 ng/mg of tissue to about 600 μg/mg of tissue, preferably from about 0.001 ng/mg of tissue to about 100 μg/mg of tissue, from about 0.1 ng/mg of tissue to about 10 μg/mg of tissue, from about 1 ng/mg of tissue to about 10 μg/mg of tissue.

Alternatively, the device of the present invention may be configured to deliver the therapeutic capable agent at a phase to a susceptible tissue site of a mammalian intracorporeal body to effectuate a mammalian tissue concentration ranging from about 0.001 ng of therapeutic capable agent/mg of tissue to about 100 μg of therapeutic capable agent/mg of tissue, usually from about 1 ng of therapeutic capable agent/mg of tissue to about 100 μg of therapeutic capable agent/mg of tissue, preferably from about 1 ng of therapeutic capable agent/mg of tissue to about 10 μg of therapeutic capable agent/mg of tissue.

The device of the present invention may further be configured to release the therapeutic capable agent at a phase to a mammalian intracorporeal body to effectuate a mammalian blood concentration ranging from about 1 ng of therapeutic capable agent/ml of blood to about 50 μg of therapeutic capable agent/ml of blood, usually from about 1 ng of therapeutic capable agent/ml of blood to about 20 μg of therapeutic capable agent/ml of blood, preferably from about 2 ng of therapeutic capable agent/ml of blood to about 12 μg of therapeutic capable agent/ml of blood. The phase may be within the first 24 hours after implantation of the device in the mammalian intracorporeal body, wherein the concentration is a peak concentration. The device may further be configured to have a termination phase delivering the therapeutic capable agent to a mammalian intracorporeal body at a rate less than a rate of clearance of the intracorporeal body of the therapeutic capable agent. The termination phase may have a duration of about 14 days. The rate of clearance is typically from about 1 ng/mg of tissue/day to about 100 ng/mg of tissue/day, usually about 80 ng/mg of tissue/day, preferably about 10 ng/mg of tissue/day.

The therapeutic capable agent as administered, may be converted to metabolites which may or may not be desirable. By way of example, when delivered systemically, mycophenolic acid (MPA) is metabolized in the blood, principally, by glucuronyl transferases to form a pharmacologically inactive phenolic glucuronide of MPA (MPAG). When MPA is delivered locally, as for example from a prosthesis such as a stent placed in the vascular system, the drug enters the tissue and coverts into MPAG, although at a different rate than that in the blood stream. If this pharamacologically inactive compound (e.g., MPAG) accumulates in the tissue, the accumulation can cause unwanted inflammation at the tissue. By way of example, a prosthesis with a therapeutic capable agent (e.g., the drug and its metabolite) present only on the tissue facing surface of the prosthesis along with a polymer coating may lead to saturation of the therapeutic capable agent, as for example an MPAG content greater than 250 ng/100 mg of tissue, resulting in localized inflammation, growth factor, cytokine generation, and excessive proliferation at the tissue. Hence, the drug delivery system should be designed in such a manner as to provide for efficient removal of MPAG from the tissue at any given point. The drug delivery system of the present invention, with MPA as the therapeutic capable agent, is designed in such a manner that the local tissue concentrations of MPA range from about 15 ng/100 mg of tissue to about 300 ng/100 mg of tissue. In an embodiment, the MPAG concentration is less than about 250 ng/100 mg of tissue, normally, less than about 110 ng/100 mg of tissue, usually less than about 50 ng/100 mg of tissue, desirably less than about 25 ng/100 mg of tissue, more preferably, less than about 10 ng/100 mg of tissue, and most desirably substantially zero.

When the device includes the source including a plurality of compounds (e.g., first therapeutic capable agent and an optional another compound such as another or second therapeutic capable agent or enabling compound), the plurality of compounds may be released at different times and/or rates, from the same or different layers. Each of the plurality of compounds may be made available independently of one another (e.g., sequential), simultaneous with one another, or concurrently with and/or subsequent to the interventional procedure. For example, a first therapeutic capable agent (e.g., TRIPTOLIDE™) may be released within a time period of 1 day to 45 days with the second therapeutic capable agent (e.g, mycophenolic acid) released within a time period of 2 days to 3 months, from the time of interventional procedure.

The expandable structure may incorporate the therapeutic capable agent and/or the optional another compound, by coating, spraying, dipping, deposition (vapor or plasma), or painting the therapeutic capable agent onto the prosthesis. Usually, the therapeutic capable agent is dissolved in a solvent. Suitable solvents include aqueous solvents (e.g., water with pH buffers, pH adjusters, organic salts, and inorganic salts), alcohols (e.g., methanol, ethanol, propanol, isopropanol, hexanol, and glycols), nitrites (e.g., acetonitrile, benzonitrile, and butyronitrile), amides (e.g., formamide and N-dimethylformamide), ketones, esters, ethers, DMSO, gases (e.g., CO₂), and the like. For example, the prosthesis may be sprayed with or dipped in the solution and dried so that therapeutic capable crystals are left on a surface of the prosthesis. Alternatively, matrix solution including a rate-sustaining or rate-controlling element material and the therapeutic capable agent may be prepared by dissolving the rate-sustaining or rate-controlling element material and the therapeutic capable agent. The expandable structure 16 may then be coated with the matrix solution by spraying, dipping, deposition, or painting the matrix onto the prosthesis. By way of example, when the matrix is formed from polymeric material, the matrix solution is finely sprayed on the prosthesis while the prosthesis is rotating on a mandrel. The thickness of the matrix coating may be controlled by the time period of spraying and a speed of rotation of the mandrel. The thickness of the matrix-agent coating is typically in a range from about 0.01 μm to about 100 μm, preferably in a range from about 0.1 μm to about 50 μm. Once the prosthesis has been coated with the matrix coating, the stent may be placed in a vacuum or oven to complete evaporation of the solvent.

In operation, methods of delivering therapeutic capable agents to a susceptible tissue site comprise providing a luminal prosthesis incorporating features of the present invention as described above. The prosthesis is delivered to a corporeal site, such as a body lumen, including the susceptible tissue site. The prosthesis is implanted within the body lumen. The therapeutic capable agent is made available to the susceptible tissue site over a period of time.

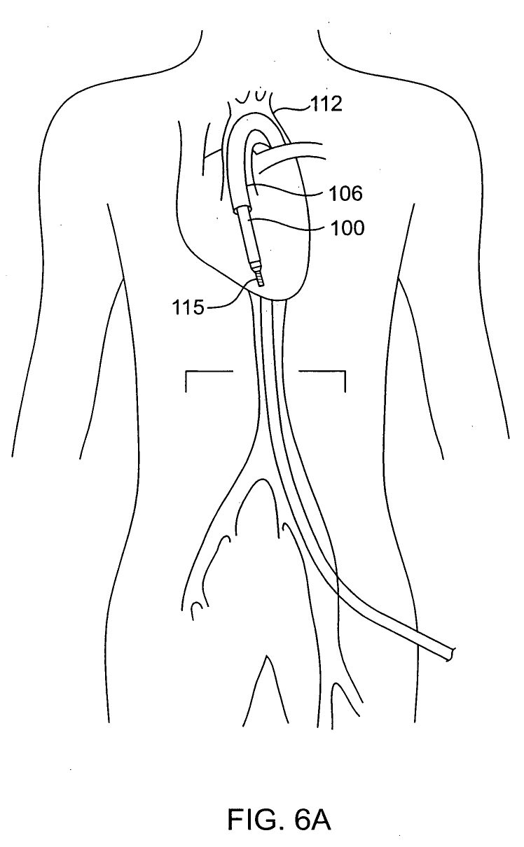

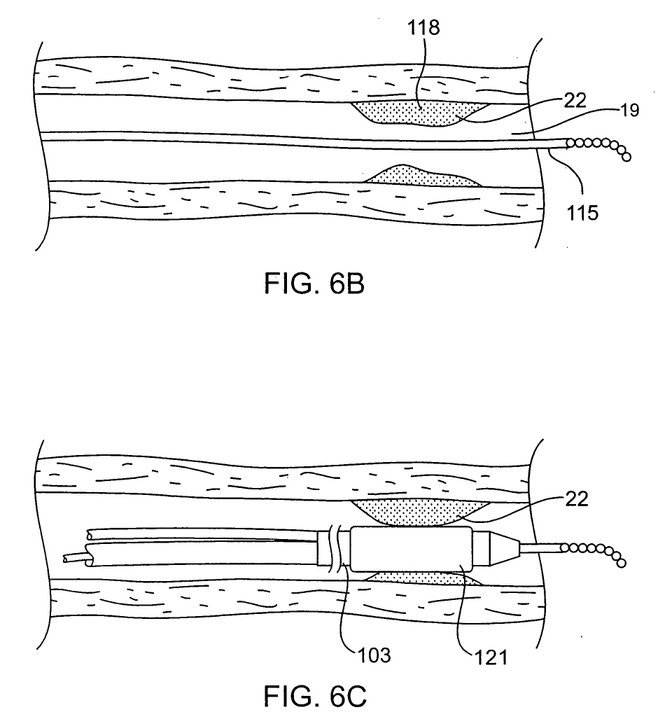

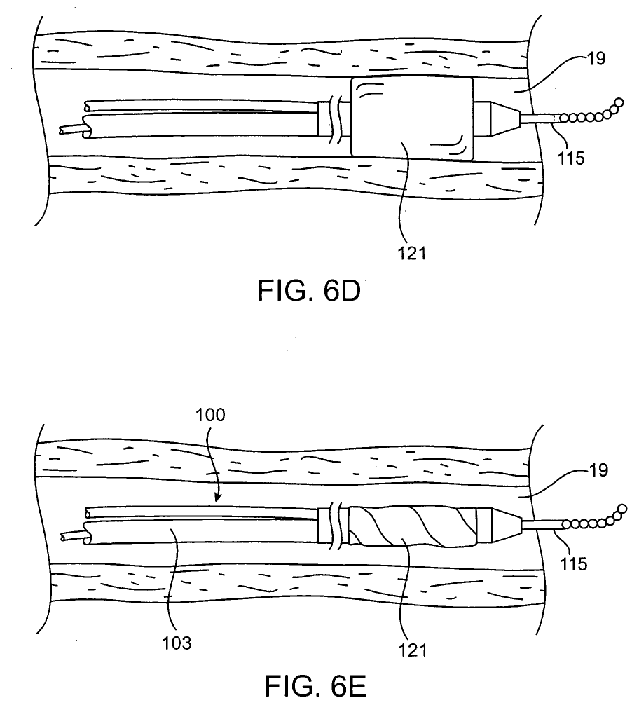

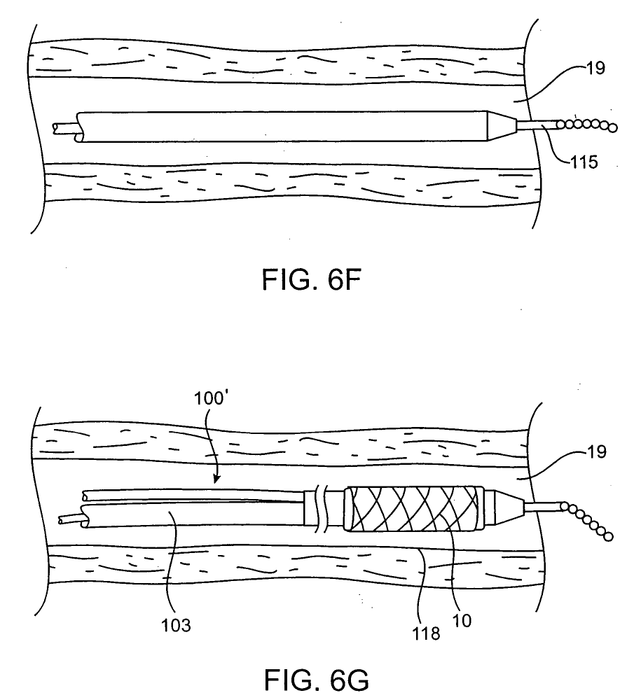

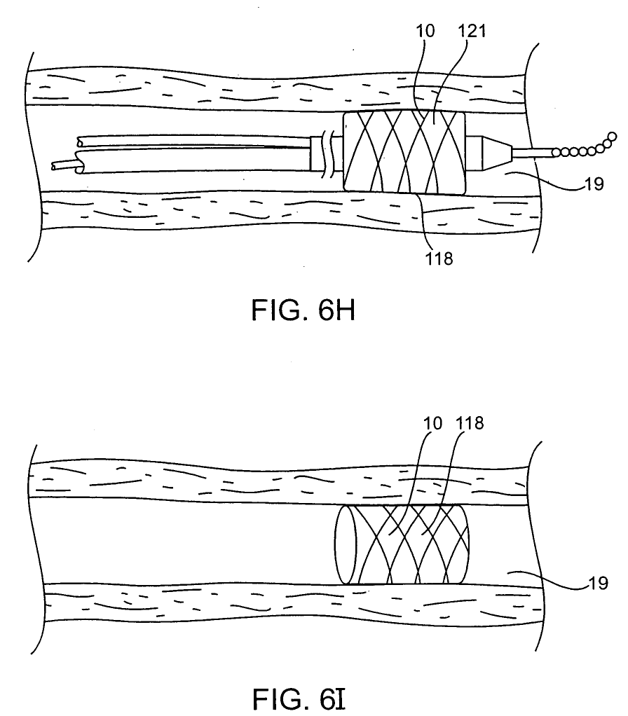

FIGS. 6A-6F, illustrate features of a method for making a therapeutic capable agent available to a susceptible tissue site. As shown in FIG. 6A, an intravasculature balloon catheter 100 having a tubular body 103 is introduced through a guiding catheter 106 via hemostatic valve and sheath (not shown) and through the femoral artery 106 to the coronary vasculature over the aortic arch 112. A guidewire 115 will usually be positioned at the target site 118 including the susceptible tissue site 22, typically a region of stenosis to be treated by balloon angioplasty (FIG. 6B). Usually, the balloon catheter 100 and guidewire 115 will be introduced together with the guidewire 115 being periodically extended distally of the catheter until the target site is reached. Once at the target site 118, a balloon 121 is inflated to expand the occlusion at the target site 118, as shown in FIGS. 6C and 6D. After the balloon angioplasty treatment is completed, the balloon 121 will be deflated, with guidewire 115 remaining in place. The balloon 121 may then be removed over guidewire 115, again with the guidewire 115 remaining in place as seen in FIGS. 6E and 6F. A second balloon assembly 100′ including a device 10 according to present invention, is then introduced over the catheter body as shown in FIG. 6G. After the second balloon assembly 100′ is in place, the device, such as stent 10 which is in place over the balloon assembly, may be deployed by inflating balloon 121 (FIG. 6H). After the stent 10 has been properly deployed, the balloon may be deflated and the catheter removed leaving the stent in place, as shown in FIG. 61. It should be appreciated that depending on the nature of the site under treatment, the device of the present invention may be introduced to the site during the introduction of the first balloon catheter without the need for pre-dilatation.

Methods of treatment generally include positioning the source including the at least one therapeutic capable agent and/or optional another compound within the intracorporeal body, concurrently with or subsequent to, an interventional treatment. More specifically, the therapeutic capable agent may be delivered to a targeted corporeal site (e.g., targeted intracorporeal site) which may include the susceptible tissue site or may provide therapeutic capable agent to the susceptible tissue site, concurrently with or subsequent to the interventional treatment. By way of example, following the dilation of the stenotic region with a dilatation balloon, a device (such as a stent) according to the present invention, is delivered and implanted in the vessel. The therapeutic capable agent may be made available to the susceptible tissue site at amounts which may be sustainable, intermittent, or continuous; at one or more phases; and/or rates of delivery.

In an embodiment, the release of the therapeutic capable agent to the susceptible tissue site may be delayed. During the delay period none to small amounts of therapeutic capable agent may be released before the release of a substantial amount of therapeutic capable agent. Typically, the delay is sufficiently long to allow for sufficient generation of intimal tissue or cellularization at the treated site to reduce the occurrence of a thrombotic event.

In one embodiment, delay is sufficiently long to allow the generated neointima to cover at least partially the implanted expandable structure. In an embodiment, the therapeutic capable agent may be released in a time period, as measured from the time of implantig of the device, ranging from about 1 day to about 200 days; from about 1 day to about 45 days; or from about 7 days to about 21 days. In an embodiment, the method further includes directing energy at the device to effect release of the therapeutic capable agent from the device. The energy may include one or more of ultrasound, magnetic resonance imaging, magnetic field, radio frequency, temperature change, electromagnetic, x-ray, heat, vibration, gamma radiation, or microwave. The total amount of therapeutic capable agent made available or released may be in an amount ranging from about 0.1 μg to about 10 g, generally about 0.1 μg to about 10 mg, usually from about 1 μg to about 10 mg, from 1 μg to about 5 mg, from about typically from about 1 μg to about 2 mg, from 10 μg to about 2 mg, from 10 μg to about 1 mg, from about 50 μg to about 1 mg, or from 50 μg to about 500 μg.

In general, it will be possible to combine elements of the differing prostheses and treatment methods as described above. For example, a prosthesis having reservoir means for releasing therapeutic capable agents may further incorporate a rate-sustaining or rate-controlling element. Additionally, methods of the present invention may combine balloon angioplasty and/or other interventional treatments to resolve a stenotic site with the presently described luminal therapeutic capable agent delivery treatments.

Non-limiting examples of the present invention are set forth below.

### EXAMPLE 1

A stainless steel DURAFLEX™ stent (available from Avantec Vascular Corporation, having a place of operation in California), having dimensions of 3.0 mm×14 mm is sprayed with a solution of 25 mg/ml therapeutic capable agent in a 100% ethanol or methanol solvent. The stent is dried and the ethanol is evaporated leaving the therapeutic capable agent on the stent surface. A 75:25 PLLA/PCL copolymer (sold commercially by POLYSCIENCES) is prepared in 1,4 Dioxane (sold commercially by ALDRICH CHEMICALS). The therapeutic capable agent loaded stent is loaded on a mandrel rotating at 200 rpm and a spray gun (sold commercially by BINKS MANUFACTURING) dispenses the copolymer solution in a fine spray on to the therapeutic capable agent loaded stent as it rotates for a 10-30 second time period. The stent is then placed in an oven at 25-35° C. up to 24 hours to complete evaporation of the solvent.

### EXAMPLE 2

A Stainless steel Duraflex stent (3.0×14 mm) was laser cut from a SS tube. The surface area of the stent for receiving the therapeutic capable agent was increased by increasing the surface roughness of the stent. The surface area and the volume of the stent can be further increased by creating 10 nm wide by 5 nm deep grooves along the links of the stent strut. The grooves were created in those stent areas experiencing low stress during expansion so as not to compromise the stent radial strength. The drug was loaded onto the stent and in the stent grooves by dipping or spraying the stent in the therapeutic capable agent solution prepared in low surface tension solvent such as isopropyl alcohol, ethanol, or methanol. The stent was then dried with the therapeutic capable agent remaining on the stent surface, and in the grooves which served as a reservoir for the therapeutic capable agent. Parylene was then vacuum deposited on the stent to serve as a rate-sustaining or rate-controlling element. The drug was eluted from the stent over a period of time in the range from 1 day to 45 days.

### EXAMPLE 3

A therapeutic capable agent was dissolved in methanol, then sprayed onto the stent. The stent was left to dry with the solvent evaporating from the stent leaving the therapeutic capable agent on the stent. A rate-sustaining or rate-controlling element (e.g., silicone, polyurethane, polytetrafluorethylene, parylene, parylene C, non-porous parylene C, PARYLAST™, PARYLAST™ C) was sprayed or deposited on the stent covering the therapeutic capable agent. The amount of therapeutic capable agent varied from about 10 micrograms to 2 milligrams, with release rates from 1 day to 45 days.

### EXAMPLE 4

A matrix solution including the matrix polymer and a therapeutic capable agent was coated onto a stent, as described in Example 2. The stent was then coated or sprayed with a top coat of a rate-sustaining or rate-controlling element (and/or a matrix material without a drug so as to act as a rate-sustaining or rate-controlling element). Alternatively, the therapeutic capable agent may be coated on a stent via a rate-sustaining or rate-controlling element, and then covered with a top coat (another element or matrix). Use of top coats provides further sustain or control of release rate, improved biocompatibility, and/or resistance to scratching and cracking upon stent delivery or expansion.

### EXAMPLE 5

The therapeutic capable agent may be combined with another or second therapeutic capable agent (cytotoxic drugs, cytostatic drugs, or psoriasis drugs). One agent is in or coupled to a first coat while other agent is in or coupled to a second coat. The first therapeutic capable agent is released a time period of 1 day to 45 days after being implanted within a vessel while the second therapeutic capable agent is released or continues to be released for a longer period.

### EXAMPLE 6

A combination of multiple therapeutic capable agents that are individually included in different coats can be used as the matrix. The coats may release the multiple agents simultaneously and/or sequentially. The agents may be selected from a therapeutic capable agent class of inhibitors of de novo nucleotide synthesis or from classes of glucocorticosteroids, immunophilin-binding drugs, deoxyspergualin, FTY720, protein drugs, or peptides. This can also apply to any combination of agents from the above classes that are coupled to a stent with the addition of other cytotoxic drugs.

### EXAMPLE 7

A matrix including the therapeutic capable agent, mycophenolic acid (at a mycophenolic acid loading of 70% to 80% by weight), and matrix polymer, CAB (cellulose acetate butyrate), was prepared by dissolving the therapeutic capable agent in acetone at 15 mg/ml concentration, dissolving CAB in acetone at 15 mg/ml concentration, and thereafter mixing together the mycophenolic acid and CAB solutions in 3:1 portion matrix solution. The amount of therapeutic capable agent varied from about 0.1 microgram to about 2 mg, preferably, at 600 microgram. The matrix solution was then coated onto two sets of stents (Sets A and B) by spraying them with an atomizer sprayer (EFD manufacturer) while each stent was rotated. Each stent was allowed to dry. One matrix-coated stent was then coated with parylene as the rate-sustaining or rate-controlling element (about 1.1 μm) using methods similar to those described in Example 2. Orifices were created on the top surface (parylene rate-sustaining or rate-controlling element) of the stents of Set B by subjecting the surface to laser beams or a needle. The orifice size can range from about 0.1 μm to about 100 μm in diameter. The orifice in Set B stent was about 10 μm in diameter. An orifice can be about 0.003 inches to about 2 inches apart from the next orifice (measured as the curvilinear distance traced along the stent strut pattern).

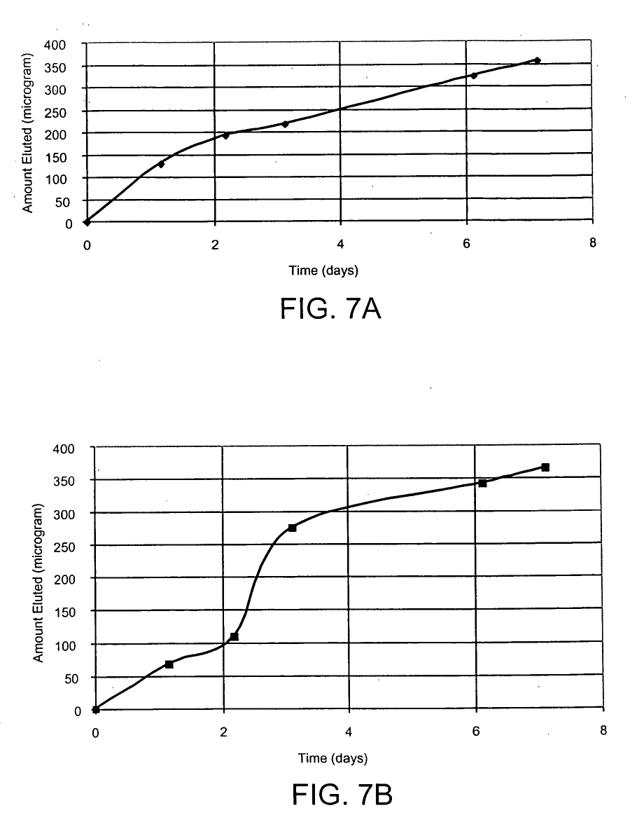

The mycophenolic acid loaded stents were placed in an elution solution of porcine serum and allowed to age for a period of 1 to 7 days. Samples from the serum were taken at regular time intervals and analyzed by HPLC. As can be seen from the data represented in FIGS. 7A and 7B (corresponding to stent sets A and B, respectively), stent Set A showed a linear release rate for the mycophenolic acid while stent Set B showed a relatively slow linear release rate at the initial phase, followed by a relatively more rapid release in the subsequent phase.

### EXAMPLE 8

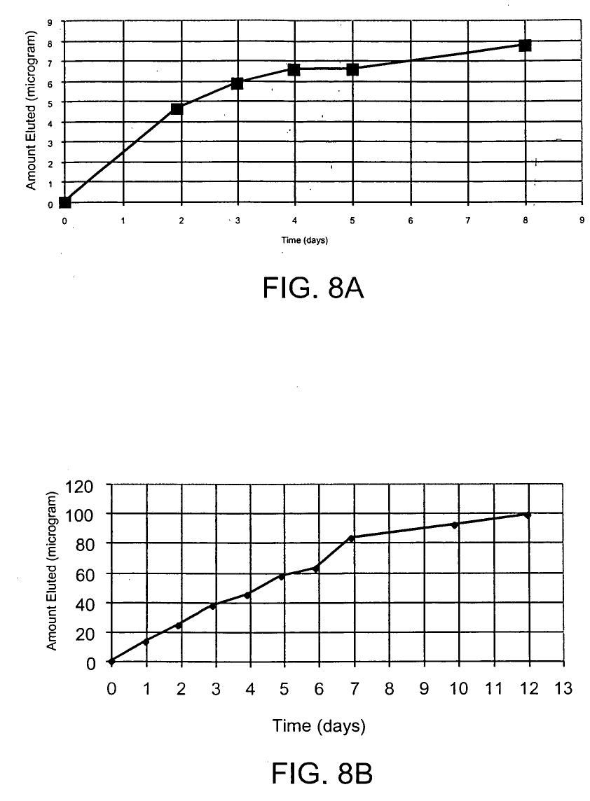

Two sets of stents, Sets A and B, were coated with 250 μg and 300 μg of mycophenolic acid, respectively, according to Example 2. Set A was then coated with 1.7 micron of parylene as the rate-sustaining or rate-controlling element. Set B was first coated with mycophenolic acid followed by a subsequent coating of methylprednisolone as the rate-limiting matrix material, and thereafter coated with 1.3 micron of parylene. The coated stents were then subjected to in vitro elution test as described in Example 7, and the amount of mycophenolic acid eluted was measured. As can be seen from the data represented in FIGS. 8A and 8B (corresponding to stent Sets A and B, respectively), both Sets showed a relatively fast linear release of the mycophenolic acid in the initial phase followed by a relatively slower release in the subsequent phase. This may suggest that the more hydrophobic methylprednisolone may act as a rate-sustaining or rate-controlling element for the more water soluble mycophenolic acid, and can act to sustain or control the release rate of mycophenolic acid along with the Parylene coating. This is useful when the diseased area needs a large bolus of the drug initially and then a sustained slower release.

### EXAMPLE 9

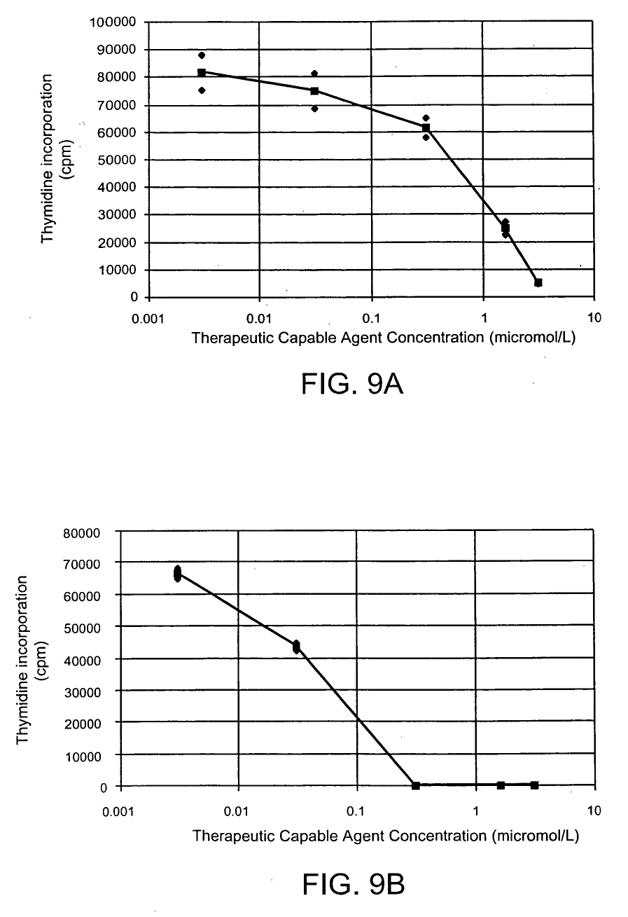

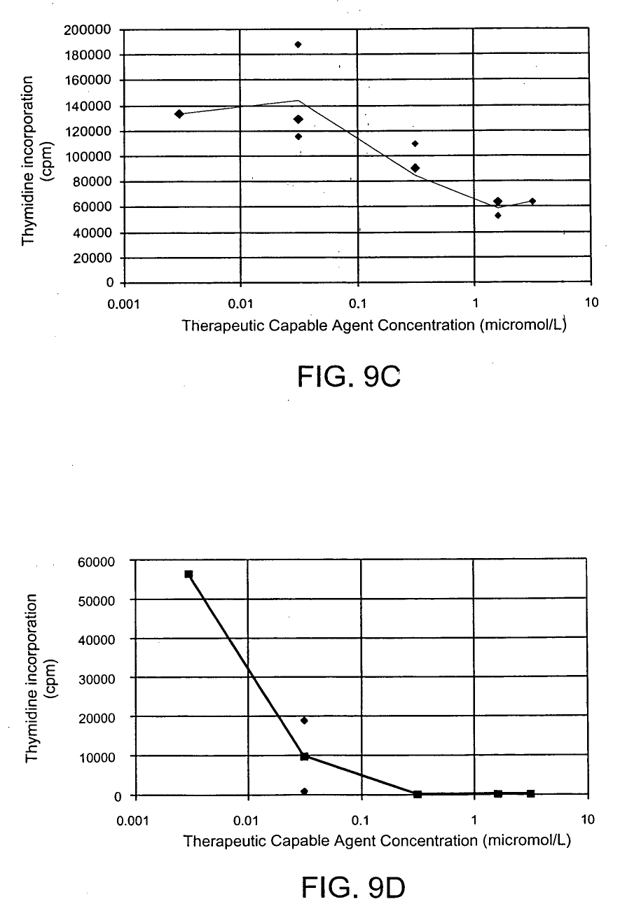

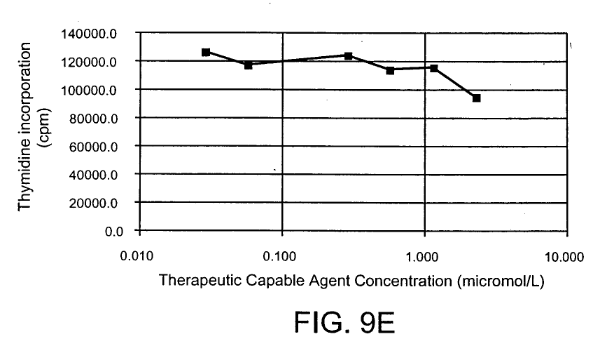

In order to assess the effect of therapeutic capable agents of the present invention on cell cultures, samples of 5 sets of therapeutic capable agents, as listed below, in varying concentrations were prepared and added to different groups of porcine smooth muscle cell cultures according to standard procedures. Set A, B, C, D, and E corresponded to therapeutic capable agent sets: Mycophenolic acid & Dexamethasone; Mycophenolic acid & TRIPTOLIDE™; WORTMANNIN™ and METHOTREXATE™; TRIPTOLIDE™, and Mycophenolate Mofetil respectively. The amount of incorporated thymidine for the different samples of varying concentrations (0.003, 0.031, 0.31, 1.6, and 3.1 micromolar) was measured. As can be seen from the data represented in FIGS. 9A-9E (corresponding to Sets A-E, respectively) the IC50 (defined as the concentration at which 50% of the cells are prevented from proliferating) for the various sets occurred at different concentrations. As can further be noted, Mycophenolate Mofetil (reference E) may not be as effective in the absence of a bio-condition (e.g., subject to bodily fluids such as blood).

### EXAMPLE 10

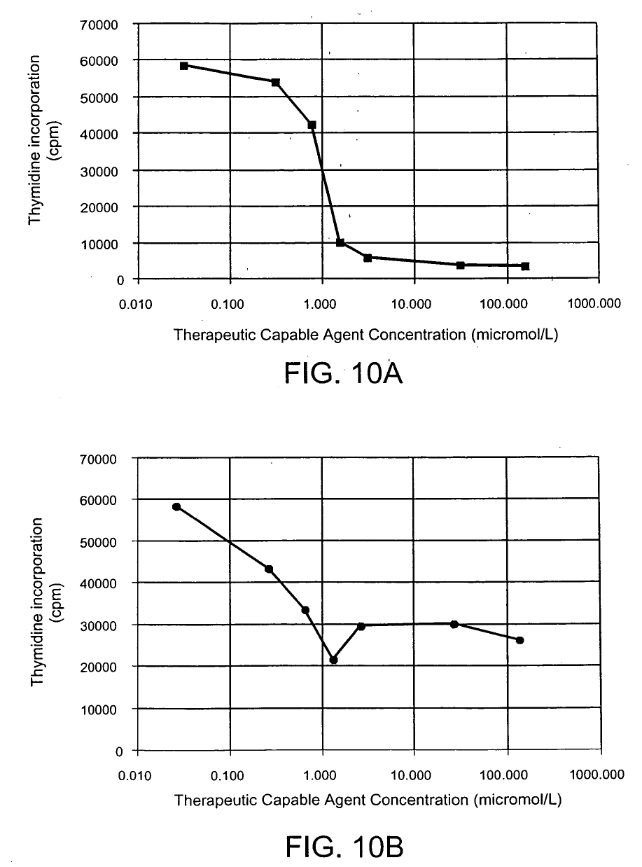

In another group of therapeutic capable agents, the amount of incorporated thymidine for samples of varying concentrations (0.003, 0.031, 0.31, 1.6, 3.1, 31, and 156 micromolar) was measured. As can be seen from the data represented in FIGS. 10A and 10B, and corresponding to Mycophenolic acid and Methylprednisolone, respectively, the IC50 for these therapeutic capable agent was 1.0 micromolar.

### EXMAPLE 11

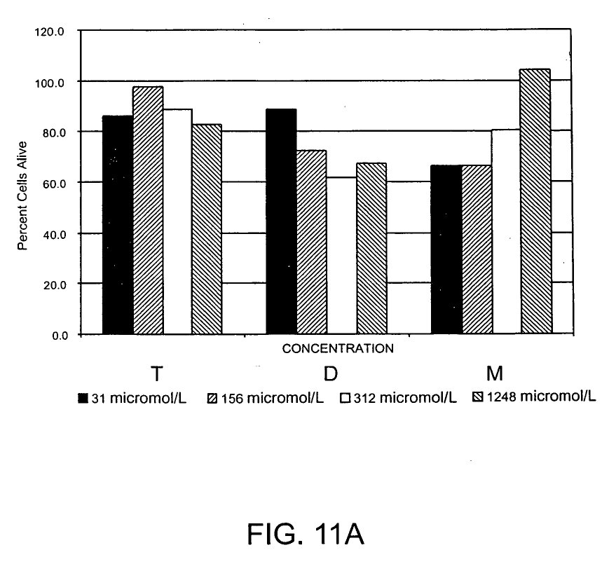

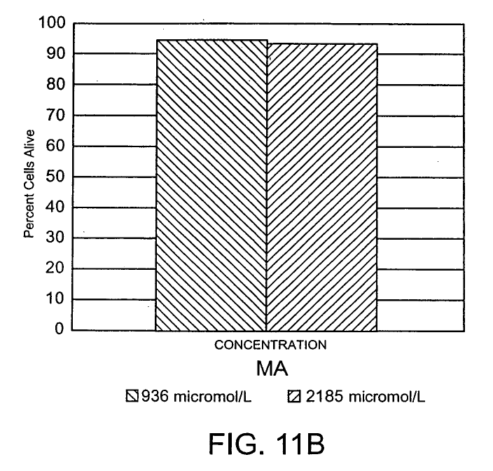

In order to assess the effect of various therapeutic capable agents, cell cultures were subjected to some therapeutic capable agents using methods similar to those described in Examples 9 and 10. As can be seen from data represented in FIGS. 11A and 11B, and corresponding, respectively, to TRIPTOLIDE™ (T); Dexamethasone (D); METHOTREXATE™ (M); and Mycophenolic Acid (MA), the therapeutic capable agents did not lead to significant cell death. In addition, it can be seen that at the IC50 concentrations, most of the cells were alive yet 50% proliferating.

### EXAMPLE 12

A therapeutic capable agent, mycophenolic acid, was prepared by dissolving the therapeutic capable agent in acetone at 15 mg/ml concentration. The amount of therapeutic capable agent varied from about 0.1 μg to about 2 mg, preferably, at 600 μg. The drug solution was then coated onto or over a stent, as described in Example 8, by spraying them with an atomizer sprayer (EFD manufacturer) while the stent was rotated. The stent was allowed to dry. The stent was then placed over the tri-fold balloon on a PTCA catheter and crimped thereon. After crimping, the drug remained intact and attached to the stent. Expansion of the stent against a simulated Tecoflex vessel showed no cracking of the drug. Exposure of fluid flow over the stent before stent deployment against the simulated vessel did not result in drug detachment from the stent.

### EXAMPLE 13

A therapeutic capable agent such as NF-κB Decoy Oligo, proteins such as albumin, genes such as TSC1, TSC2, hamartin KIAA0243, growth factors such as VEGF, EGF, PDGF, FGF, anti-sense such as Antisense phosphorothioate oligodeoxynucleotide (ODN), anti-bodies such as Anti-MTOR, Anti-p27 Anti-p53, Anti-Cdk was dissolved in saline, then sprayed onto the stent. The stent was left to dry in a low 14MTORr vacuum until water evaporated from the stent leaving the therapeutic capable agent on the stent. A rate-sustaining or rate-controlling element (e.g., parylene, parylene C, non-porous parylene C, PARYLAST™, PARYLAST™ C with a foam structure, porous structure, nano-porous structure, non-porous structure, structure with cracks, openings, fissures, perforations, other before or after positioning in tissue/physiological fluid, or a combination thereof) was deposited on the stent adjacent to the therapeutic capable agent. The amount of therapeutic capable agent varied from about 1 micrograms to 2 milligrams, with release rates from 1 hr to 365 days.

### EXAMPLE 14

In order to evaluate the effect of therapeutic capable agent coating configuration on tissue concentration of MPEG, two groups of stents were loaded, each with 300 μg mycophenolic acid as the therapeutic capable agent. In loading the stents of Group 1 with the therapeutic capable agent, only one of the two longitudinal surfaces, namely the tissue facing surface was loaded. To load the stent only on the tissue facing surface and not the luminal surface, a Teflon mandrel was snugly fit inside the luminal area of the stent and the stent was thereafter loaded with the therapeutic capable agent using a spray process as previously described. The other group was loaded with the therapeutic capable agent on both surfaces, using the same process but without the presence of the Teflon mandrel. The Teflon mandrel was then removed from the stents of group one, and both groups were coated with a 1.9 μm Parylene coating as described earlier.

The coated stents were then loaded on a catheter delivery system, sterilized, and tested in vivo using a 28 day porcine coronary artery model. The coronary tissue was explanted along with the stent and used to perform histology and histomorphometric analysis. A small group of animals were sacrificed at shorter time periods of 7 days to perform pharmacokinetic analysis. The tissue from these animals was tested for MPA and MPAG content.

The histology results indicated that there was more inflammation in the vessel wall of the pigs which received the stents of group one (one sided). This could have been explained if there were excessive drug in the tissue, since all the 300 μg dose would be available to the tissue and the dose experienced by the cells in this case would have been very high. However, the tissue concentrations from the vessel walls for both the groups presented similar concentrations of MPA. There was also higher amounts of MPAG in the tissue of the vessels which received the stents of group one. The amounts of MPA and MPAG are presented in Table I below. As can be seen from the results, it is believed that MPAG concentrations found in the vessel tissue could lead to inflammation and reduce the therapeutic effect of MPA.

TABLE I

| Coating | MPA concentration | MPAG concentration |
|---|---|---|
| Configuration | (ng/stented vessel tissue) | (ng/stented vessel tissue) |
| 300 μg MPA (one | 175 ± 73 ng | 366 ± 140 ng |
| sided) with 1.9 μm | | |
| Parylene coating | | |
| 300 μg MPA (two | 143 ± 43 ng | <50 ng (detection limit) |
| sided) with 1.9 μm | | |
| Parylene coating | | |

On the other hand, the stents of group 2 (drug loaded on both sides) presented less amount of MPA to the tissue and hence less MPAG. Based on further evaluation of the stents, it is believed that the MPA on the luminal side becomes covered by plasma proteins and eventually by fibrin deposition which can then serve as a depot for MPA and hence can keep the MPA concentration in the tissue at therapeutic levels without significant amounts of inflammatory MPAG. It is believed that the one sided coated stents could present more drug to the tissue, however this may also lead to saturation of the tissue with MPA and MPAG, with the glucoronic acid in the tissue converting MPA to MPAG, and the entrapped MPAG causing inflammation.

The process of loading a therapeutic capable agent like MPA can be used to sustain or control not only the amount of therapeutic capable agent in the susceptible tissue site but also the by-products and derivatives of the drug in the region.

Although certain preferred embodiments and methods have been disclosed herein, it will be apparent from the foregoing disclosure to those skilled in the art that variations and modifications of such embodiments and methods may be made without departing from the true spirit and scope of the invention. Therefore, the above description should not be taken as limiting the scope of the invention which is defined by the appended claims.

1. A device for intracorporeal use, the device comprising:
an expandable structure; and
at least one source of at least one therapeutic capable agent associated with the structure, wherein the at least one therapeutic capable agent is selected from the group consisting of IGF, IGF-1, IGFBP, IGFBP-3, and rhlGFBP-3.

2. The device of claim 1, wherein the device is configured to release the at least one therapeutic capable agent to a body lumen or organ within an intracorporeal body to inhibit smooth muscle cell proliferation.

3. The device of claim 1, wherein the expandable structure comprises a stent, graft, or a scaffold formed at least in part from an open lattice.

4. The device of claim 1, wherein the expandable structure has a luminal and a tissue facing surface, and wherein the therapeutic capable agent is associated with at least one of the luminal or tissue facing surfaces.

5. The device of claim 1, wherein source further includes at least another compound.

6. The device of claim 5, wherein the device is configured to release the another compound prior to, concurrent with, or subsequent to the release of the therapeutic capable agent.

7. The device of claim 5, wherein the at least another compound is selected from the group consisting of immunosuppressants, anti-inflammatories, anti-proliferatives, anti-migratory agents, anti-fibrotic agents, proapoptotics, vasodilators, calcium channel blockers, anti-neoplastics, anti-cancer agents, antibodies, anti-thrombotic agents, anti-platelet agents, IIb/IIIa agents, antiviral agents, MTOR (mammalian target of rapamycin) inhibitors, non-immunosuppressant agents, tyrosine kinase inhibitors, EGFR/ErbB2 inhibitors, VEGF receptor inhibitors, VEGFR/FGFR/PDGFR inhibitors, NGF receptor inhibitors, anti-EGF receptor MAbs, anti-ErbB2 MAbs, CDK inhibitors, bisphosphonates, NF-κB Decoy Oligo, proteins, oligomers, amino acids, peptides, genes, growth factors, anti-sense, and a combination thereof.

8. The device of claim 5, wherein the at least another compound is selected from the group consisting of mycophenolic acid; mycophenolic acid derivatives including 2-methoxymethyl derivative, 2-methyl derivative, and sodium mycophenolic acid; VX-148; VX-944; mycophenolate mofetil; mizoribine; methylprednisolone; dexamethasone; rapamycin; deuterated rapamycin; rapamycin analogs or derivatives including AP23573, RAPALOGS™ including AP21967, CERTICAN™, 32-deoxorapamycin, ABT-578, CCI-779; ABT-773; ABT-797; TRIPTOLIDE™; METHOTREXATE™; phenylalkylamines including verapamil; benzothiazepines including diltiazem; 1,4-dihydropyridines including benidipine, nifedipine, nicarrdipine, isradipine, felodipine, amlodipine, nilvadipine, nisoldipine, manidipine, nitrendipine, barnidipine; ASCOMYCIN™; PIMECROLIMUS™; WORTMANNIN™; LY294002; CAMPTOTHECIN™; silibinin; sylymarin; baicalein; histone deacetylase including trichostatin A; PD-0183812; butyrolactone I substituted purines including olomoucine, CGP74514, and its derivatives; polyhydroxylated flavones including flavopyridol; oxindole inhibitors including GW-8510, GW-2059, GW-5181; indolinone derivatives including SU-5416; Zoledronic acids including ZOMETA™, Zoledronate, and (1-Hydroxy-2-imidazol-1-yl-phosphonoethyl)phosphonic acid monohydrate; isoquinoline; HA-1077 (1-(5-isoquinolinesulfonyl)-homopiperazine hydrochloride); TAS-301; TOPOTECAN™; hydroxyurea; TACROLIMUS™; cyclophosphamide; cyclosporine; daclizumab; azathioprine; prednisone; diferuloymethane; diferuloylmethane; diferulylmethane; GEMCITABINE™; cilostazol; TRANILAST™; enalapril; quercetin; suramin; estradiol; cycloheximide; tiazofurin; zafurin; benidipine hydrochloride; phenylaminopyrimidine derivatives including Imatinib mesylate; other tyrosine inhibitors such as 4-[6-methoxy-7-(3-piperidine-1-yl-propoxy)-quinazolin-4-yl]-piperazine-1-carboxylicacid(4-isopropoxyphenyl)amide (CT53518 or MLN518 from Millennium Pharmaceutical), 5-Chloro-3-[(3,5-dimethylpyrrol-2-yl)methylene]-2-indolinone (SU6656), 5-Chloro-3-[(3,5-dimethylpyrrol-2-yl)methylene]-2-indolinone (SU5614 from Sugen), a water-soluble N,N-dimethylgly-cine ester prodrug CEP7055 that converts to CEP5214 in vivo from Cephalon, West Chester Pa., 4-Amino-5-(4-chlorophenyl)-7-(t-butyl)pyrazolo[3,4-d]pyrimidine (PP2 or AG1879), 6,7-Dimethyl-2-phenylquinoxaline (AG1295), TAUTOMYCIN™, Radicicol, Damnacanthal, Herbimycin A, 6-(2,6-dichloro-phenyl)-8-methyl-2-(3-methylsulfanyl-phenylamino)-8h-pyrido(2,3-d)pyrimidin-7-one (PD173955 from Parke-Davis), PD166326, PD183805, 4-[(3-Bromophenyl)amino]-6-propionylamidoquinazoline (PD174265), 5-Chloro-3-[(3,5-dimethylpyrrol-2-yl)methylene]-2-indolinone (PD153035), 4-[(3-Bromophenyl)amino]-6-acrylamidoquinazoline (PD168393), TARCEVA™ (erlotinib HCl), CI-1033, AEE788, CP-724,714 (from OSI Pharmaceutical), Geldanamycin, 17-(allylamino)-17-demethoxygeldanamycin (17-AG or 12-AAG), TARCEVA™, IRESSA™, and ZD4910, EGFR/ErbB2 inhibitor (CI1033; EKB569; GW2016; PKI166), VEGF receptor inhibitors (ZK222584;ZD6474), VEGFR/FGFR/PDGFR inhibitors (SU6668; SU11248; PTK787), NGF receptor inhibitors (CEP2583), anti-EGF receptor MAbs (MAb225/ERBITUX™), anti-ErbB2 MAbs (MAb4D5/HERCEPTIN™), AVASTIN™, an anti-VEGF MAb, bisphosphonates; NF-κB Decoy oligonucleotides; proteins including albumin; genes including TSC1, TSC2, hamartin, and KIAA0243; growth factors including VEGF, EGF, PDGF, and FGF; anti-sense including antisense phosphorothioate oligodeoxynucleotide; anti-bodies including anti-MTOR, anti-p27, anti-p53, and anti-Cdk; metabolites and derivatives thereof; therapeutic capable agents incorporated in a vector including HVJ Envelop vector; and combinations thereof.

9. The device of claim 1, wherein the at least one therapeutic capable agent includes an active compound, a pro-drug of the active compound, a metabolite of the active compound, a derivative of the active compound, an analogue of the active compound, or a combination thereof.

10. The device of claim 1, wherein the device is configured to release the therapeutic capable at a release rate so as to provide a sustainable level of therapeutic capable agent to a susceptible tissue site.

11. The device of claim 10, wherein the release rate is substantially constant, decreasing over time, increasing over time, or substantially non-releasing.

12. The device of claim 1, wherein the device is configured to release the at least one therapeutic capable agent at a rate between about 0.001 μg/day to about 500 μg/day.

13. The device of claim 1, wherein the device is configured to release the therapeutic capable agent at a total amount ranging from about 0.1 μg to about 10 g.

14. The device of claim 1, wherein the device is configured to release the therapeutic capable agent within a time period from about one day to about 200 days.

15. The device of claim 1, wherein the device is configured to release the therapeutic capable agent at an initial phase having an initial rate of release ranging from about 0 to about 99% of a subsequent rate of release of a subsequent phase.

16. The device of claim 1, wherein the device is configured to release the therapeutic capable agent at an initial phase having an initial rate of release ranging from about 0 to about 50 μg/day, and a subsequent phase having a subsequent rate of release ranging from about 0.01 μg/day to about 200 μg/day.

17. The device of claim 1, wherein the device is configured to release the therapeutic capable agent at an initial phase having an initial rate of release ranging from about 10 μg/day to about 300 μg/day, and a subsequent phase having a subsequent rate of release ranging from about 0.1 μg/day to about 100 μg/day.

18. The device of claim 1, wherein the device is configured to release the therapeutic capable agent at an initial phase having a time duration of less than about 24 weeks.

19. The device of claim 1, wherein the device is configured to release the therapeutic capable agent at a subsequent phase having a time duration in a range from about 1 hour to about 50 weeks.

20. The device of claim 1, wherein the device is configured to release the at least one therapeutic capable agent at a substantially constant rate ranging from about 0.01 μg/day to about 200 μg/day.

21. The device of claim 1, wherein the device is configured to deliver the therapeutic capable agent at a phase to a susceptible tissue site of a mammalian intracorporeal body to effectuate a mammalian tissue concentration ranging from about 0.001 ng of therapeutic capable agent/mg of tissue to about 100 μg of therapeutic capable agent/mg of tissue.

22. The device of claim 1, wherein the device is configured to release the therapeutic capable agent at a phase to a mammalian intracorporeal body to effectuate a mammalian blood concentration ranging from about 1 ng of therapeutic capable agent/ml of blood to about 50 μg of therapeutic capable agent/ml of blood.

23. The device of claim 1, wherein the device is configured to deliver the at least one therapeutic capable agent to a susceptible tissue site of a mammalian intracorporeal body to effectuate an unwanted metabolite of the therapeutic capable agent having a mammalian tissue concentration of less than 2.5 ng/mg of tissue.

24. The device of claim 1, wherein the source is configured to delay release of the therapeutic capable agent, wherein the delay is sufficiently long to allow formation of a sufficient amount of cellularization, endothelization, or fibrin deposition at a susceptible tissue site or on the device.

25. The device of claim 1, further comprising a rate-controlling element disposed adjacent at least a portion of the source or the expandable structure.

26. The device of claim 25, wherein at a least a portion of the rate-controlling element forms a matrix with the therapeutic capable agent.

27. The device of claim 25, wherein at least a portion of the rate-controlling element forms a layer adjacent at least a portion of the therapeutic capable agent.

28. The device of claim 25, wherein the therapeutic capable agent is released by surface degradation, bulk degradation, diffusion, or hydrolysis of the rate-controlling element.

29. The device of claim 25, wherein the rate-controlling element is formed from a material selected from the group consisting of parylene, parylast, polyurethane, polyethylenes imine, cellulose acetate butyrate, ethylene vinyl alcohol copolymer, silicone, polytetrafluorethylene (PTFE), poly(methyl methacrylate butyrate), poly-N-butyl methacrylate, poly(methyl methacrylate), poly 2-hydroxy ethyl methacrylate, poly ethylene glycol methacrylates, poly vinyl chloride, poly(dimethyl siloxane), poly(tetrafluoroethylene), poly(ethylene oxide), poly ethylene vinyl acetate, poly carbonate, poly acrylamide gels, N vinyl-2-pyrrolidone, maleic anhydride, Nylon, cellulose acetate butyrate (CAB) and the like, including other synthetic or natural polymeric substances; mixtures, copolymers, and combinations thereof.

30. The device of claim 25, wherein the rate-controlling element has thickness ranging from about 10 nm to about 100 μm.

31. The device of claim 1, wherein the source comprises a matrix including the therapeutic capable agent.

32. The device of claim 1, wherein the source is a polymeric material including the therapeutic capable units associated with a polymeric or metallic backbone.

33. The device of claim 1, wherein the source is a reservoir disposed adjacent the expandable structure.

34. The device of claim 1, wherein the device is configured to release the therapeutic capable agent in response to an external source of energy.

35. The device of claim 1, wherein the therapeutic capable agent is made available to a susceptible tissue site as the expandable structure degrades within the intracorporal body over time.

36. The device of claim 1, wherein the device further comprises a bio-compatible outer layer.

37. The device of claim 1, wherein the source is associated with the expandable structure by coating, spraying, dipping, vapor deposition, plasma deposition, or painting of the source onto or in the expandable structure.

38. A method for treatment of a patient, the method comprising:
providing a vascular prosthesis comprising a structure and at least one source of at least one therapeutic capable agent associated with the structure;
implanting the vascular prosthesis within the patient's vasculature including a susceptible tissue site; and
releasing the at least one therapeutic capable agent, wherein the at least one therapeutic capable agent is selected from the group consisting of IGF, IGF-1, IGFBP, IGFBP-3, and rhlGFBP-3.
